(19) United States
(12) Patent Application Publication
(10) Pub. No.: US 2025/0040546 A1
(43) Pub. Date: Feb. 6, 2025

(54) DETERGENT FOR VIRAL INACTIVATION

(71) Applicant: Bristol-Myers Squibb Company, Princeton, NJ (US)

(72) Inventors: Hasin Maksura FEROZ, Waltham, MA (US); Yuanyuan JI, Watertown, MA (US); Melissa Ann HOLSTEIN, Westford, MA (US); Sanchayita GHOSE, Acton, MA (US); Jin YU, Sherman Oaks, CA (US); Zhi LI, Shrewsbury, MA (US); Dong YANG, Belmont, MA (US); Brian James WALSH, Newtown, PA (US)

(21) Appl. No.: 18/719,403

(22) PCT Filed: Dec. 16, 2022
(86) PCT No.: PCT/US2022/081851

§ 371 Date: Jun. 13, 2024

Related U.S. Application Data
(60) Provisional application No. 63/290,470, filed on Dec. 16, 2021.

(51) Int. Cl. A01N 43/16; A01P 1/00; A61K 38/17

(52) U.S. Cl. CPC A01N 43/16; A01P 1/00; A61K 38/1774

(57) ABSTRACT

The present disclosure provides compositions and methods of inactivating a lipid enveloped virus in a product feedstream in a manufacturing process of a therapeutic protein using environmentally compatible detergents. The present disclosure provides a detergent mixture comprising two environmentally sustainable detergents: n-Octyl-β-D-Glucopyranoside (OG) and n-Dodecyl-β-D-Maltopyranoside (DDM). The performance of this OG:DDM detergent combination is superior to that of Lauryldimethylamine Oxide (LDAO), ECOSURF™ EH9, or Triton X-100 in the purification of therapeutic proteins such as abatacept and belatacept. OG:DDM combinations are highly effective for viral inactivation, while having essentially no impact on protein stability, protein charge distribution (e.g., sialic acid levels), impurity clearance, protein deamination, or protein oxidation. Accordingly, combinations of OG and DDM can be used as substitutes of Triton X-100 for viral inactivation steps in the production of biologics.

### CROSS REFERENCE TO RELATED APPLICATIONS

This PCT application claims the priority benefit of U.S. Provisional Application No. 63/290,470 filed Dec. 16, 2021, which is incorporated herein by reference in its entirety.

### REFERENCE TO SEQUENCE LISTING SUBMITTED ELECTRONICALLY VIA EFS-WEB

The content of the electronically submitted sequence listing (Name: 3338_284PC01_Seqlisting_ST26, Size: 6,037 bytes; and Date of Creation: Dec. 15, 2022) submitted in this application is incorporated herein by reference in its entirety.

### FIELD

Methods of inactivating viruses in product feed stream in a manufacturing process of a recombinant protein using two environmentally compatible detergents used in combination.

### BACKGROUND

Protein viral contaminants are a major concern in the biopharmaceutical industry in the manufacture of therapeutics of human/animal origin, which include recombinant protein, antibodies, plasma derived immunoglobulins, hormones, or microorganism-derived products such as vaccines. Most biologics manufacturing processes need to effectively remove these potential contaminants to ensure safe administration of therapeutics to patients, and to comply with regulatory requirements. With over 80% of current biologics, e.g., antibodies and recombinant proteins being produced by CHO (Chinese Hamster Ovary) cells, FDA (Food and Drug Administration) imposes stringent guidelines to show robust virus clearance in the manufacture of therapeutics of mammalian origin.

Viral contamination may arise from the cell line itself when cells produce viruses endogenously or have latent or persistent infection. Alternatively, virus may be introduced during the recombinant production process due to the use of contaminated reagents or viral vectors. As a safety measure, recombinant protein production is tested for contamination of viruses in cell lines, raw materials, and products in different stages of the downstream process in addition to carrying out viral clearance studies at different unit operation steps. The mode of virus clearance is highly dependent on the structure of viral contaminants, which may be either lipid-enveloped or non-lipid-enveloped. While filtration and chromatographic steps can effectively remove both types, chemical inactivation using low pH, detergents, solvent/detergent mixture, or other chemicals is only effective for lipid-enveloped viruses.

### BRIEF SUMMARY

The present disclosure provides a method of inactivating a virus in a product feedstream in a manufacturing process of a therapeutic protein, comprising contacting the product feedstream with n-Dodecyl-β-D-Maltopyranoside (DDM). In some aspects, the method further comprises contacting the product feedstream with n-Octyl-β-D-Glucopyranoside (OG). Also provided is a method of inactivating a virus in a product feedstream in a manufacturing process of a therapeutic protein comprising contacting the feedstream with a composition comprising DDM and OG.

In some aspects, the DDM is present at a concentration which is at least about 1, at least about 2, at least about 3, at least about 4, at least about 5, at least about 6, at least about 7, at least about 7.5, at least about 8, at least about 9, at least about 10, at least about 11, at least about 12, at least about 13, at least about 14, at least about 15, at least about 16, at least about 17, at least about 18, at least about 19, or at least about 20 times its critical micelle concentration (CMC). In some aspects, the DDM is present at a concentration which is between about 1 to about 20 times its CMC, between about 1 to about 19 times its CMC, between about 1 to about 18 times its CMC, between about 1 to about 17 times its CMC, between about 1 to about 16 times its CMC, between about 1 to about 15 times its CMC, between about 1 to about 14 times its CMC, between about 1 to about 13 times its CMC, between about 1 to about 12 times its CMC, between about 1 to about 11 times its CMC, between about 1 to about 10 times its CMC, between about 1 to about 9 times its CMC, between about 1 to about 8 times its CMC, between about 1 to about 7 times its CMC, between about 1 to about 6 times its CMC, between about 1 to about 5 times its CMC, between about 1 to about 4 times its CMC, between about 1 to about 3 times its CMC, or between about 1 to about 2 times its CMC. In some aspects, the DDM is present at a concentration between about 5 to about 10 times its CMC.

In some aspects, the OG is present at a concentration which is at least about 0.1 times its CMC, at least about 0.2 times its CMC, at least about 0.3 times its CMC, at least about 0.4 times its CMC, at least about 0.5 times its CMC, at least about 0.6 times its CMC, at least about 0.7 times its CMC, at least about 0.8 times its CMC, at least about 0.9 times its CMC, or at least about 1 time its CMC.

In some aspects, the OG is present at a concentration between about 0.1 to about 1 times its CMC. In some aspects, the OG is present at a concentration which is about 0.5 times its CMC, and DDM is present at a concentration which is between about 5 to about 10 times its CMC.

In some aspects, (i) the OG is present at a concentration which is about 0.5 times its CMC, and the DDM is present at a concentration which is about 5 times its CMC, (ii) the OG is present at a concentration which is about 0.5 times its CMC, and the DDM is present at a concentration which is about 7.5 times its CMC, (iii) the OG is present at a concentration which is about 0.5 times its CMC, and the DDM is present at a concentration which is about 10 times its CMC, or (iv) the OG is present at a concentration which is about 0.75 times its CMC, and the DDM is present at a concentration which is about 5 times its CMC.

In some aspects, the product feedstream comprises a harvest from a bioreactor, a chromatography load, a chromatography eluate, a filtration load, a filtrate, or any combination thereof. In some aspects, the chromatography eluate is a Protein A chromatography eluate. In some aspects, the virus comprises a lipid-enveloped virus. In some aspects, the lipid-enveloped virus is a retrovirus. In some aspects, the retrovirus is A-MuLV. In some aspects, the lipid-enveloped virus is a herpesvirus. In some aspects, the herpesvirus is HSV-1.

In some aspects, inactivating the lipid-enveloped virus comprises a log reduction value (LRV) of at least about 4, at least about 5, at least about 6, at least about 7, at least about 8, at least about 9, or at least about 10, and wherein the LRV is calculated as:

$$LRV = - {log}_{10} \frac{{[ Virus in pfu ]}_{P r o d u c t p o o l}}{{[ Virus in pfu ]}_{L o a d / F e e d}} .$$

In some aspects, the LRV is at least about 4. In some aspects, the contacting occurs for at least about 15 minutes, at least about 30 minutes, at least about 60 minutes, at least about 70 minutes, at least about 80 minutes, at least about 90 minutes, at least about 100 minutes, at least about 110 minutes, or at least about 120 minutes.

In some aspects, the product feedstream contains an amount of high molecular weight (HMW) species of the therapeutic protein after the contacting below about 30%, below about 29%, below about 28%, below about 27%, below about 26%, below about 25%, below about 24%, below about 23%, below about 22%, below about 21%, below about 20%, below about 19%, below about 18%, below about 17%, below about 16%, below about 15%, below about 14%, below about 13%, below about 12%, below about 11%, below about 10%, below about 9%, below about 8%, below about 7%, below about 6%, or below about 5% of the total amount of therapeutic protein.

In some aspects, the therapeutic protein has an amount of glycosylation after the contacting which is the same or changed (increased or decreased) by about 10%, about 9%, about 8%, about 7%, about 6%, about 5%, about 4%, about 3%, about 2%, or about 1% compared to the amount of glycosylation of the therapeutic protein prior to the contacting.

In some aspects, the therapeutic protein has an amount of N-acetylneuraminic acid (NANA) between about 8 to about 12 moles/mole therapeutic protein, and/or an amount of N-glycolylneuraminic acid (NGNA) less than or equal to about 1.3 moles/mole therapeutic protein after the contacting.

In some aspects, the therapeutic protein has an amount of deamidation of after the contacting of less about 5.9% of the total amount of therapeutic protein.

In some aspects, the therapeutic protein has an amount of oxidation after the contacting after the contacting of less about 1.3% of the total amount of therapeutic protein.

In some aspects, the product feedstream has a residual amount of host cell proteins after the contacting at a concentration of less than about 5,000 ppm, less than about 4,000 ppm, less than about 3,000 ppm, less than about 2,000 ppm, less than about 1,500 ppm, less than about 1,000 ppm, less than about 900 ppm, less than about 800 ppm, less than about 700 ppm, less than about 600 ppm, or less than about 500 ppm. In some aspects, the residual amount of host cell proteins in the product feedstream after the contacting is at a concentration between about 500 ppm and about 2,000 ppm.

In some aspects, the product feedstream has a residual amount of DNA after the contacting at a concentration of less than about 80,000 ppb, less than about 75,000 ppb, less than about 70,000 ppb, less than about 65,000 ppb, less than about 60,000 ppb, less than about 59,000 ppb, less than about 58,000 ppb, less than about 57,000 ppb, or less than about 56,000 ppb. In some aspects, the product feedstream has a residual amount of DNA after the contacting at a concentration of less than about 500 ppb, less than about 450 ppb, less than about 400 ppb, less than about 350 ppb, less than about 300 ppb, less than about 250 ppb, or less than about 200 ppb. In some aspects, the residual amount of DNA in the product feedstream after the contacting is between about 50 and about 200 ppb.

In some aspects, the product feedstream has a residual amount of Protein A after the contacting of less than about 1.0 μg/mL, about 0.9 μg/mL, about 0.8 μg/mL, about 0.7 μg/mL, about 0.6 μg/mL, about 0.5 μg/mL, about 0.4 μg/mL, about 0.3 μg/mL, or about 0.2 μg/mL.

In some aspects, the therapeutic protein comprises an antibody, antibody fragment, a fusion protein, a naturally occurring protein, a chimeric protein, or any combination thereof. In some aspects, the therapeutic protein comprises a CTLA4 domain. In some aspects, the therapeutic protein is a fusion protein. In some aspects, the fusion protein comprises an Fc portion. In some aspects, the therapeutic protein is abatacept or belatacept. In some aspects, the therapeutic protein is an abatacept composition comprising an amino acid sequence as set forth in SEQ ID NO:3, a fragment thereof, or a combination thereof. In some aspects, the therapeutic protein is a belatacept composition comprising an amino acid sequence as set forth in SEQ ID NO:4, a fragment thereof, or a combination thereof.

The present disclosure also provides a composition for inactivating a virus in a product feedstream in a manufacturing process of a therapeutic protein, wherein the composition comprises n-Dodecyl-β-D-Maltopyranoside (DDM). In some aspects, the composition further comprises n-Octyl-β-D-Glucopyranoside (OG).

Also provided is composition of inactivating a virus in a product feedstream in a manufacturing process of a therapeutic protein, wherein the composition comprises DDM and OG. In some aspects, the DDM is present at a concentration which is at least about 1, at least about 2, at least about 3, at least about 4, at least about 5, at least about 6, at least about 7, at least about 7.5, at least about 8, at least about 9, at least about 10, at least about 11, at least about 12, at least about 13, at least about 14, at least about 15, at least about 16, at least about 17, at least about 18, at least about 19, or at least about 20 times its critical micelle concentration (CMC). In some aspects, the DDM is present at a concentration which is between about 1 to about 20 times its CMC, between about 1 to about 19 times its CMC, between about 1 to about 18 times its CMC, between about 1 to about 17 times its CMC, between about 1 to about 16 times its CMC, between about 1 to about 15 times its CMC, between about 1 to about 14 times its CMC, between about 1 to about 13 times its CMC, between about 1 to about 12 times its CMC, between about 1 to about 11 times its CMC, between about 1 to about 10 times its CMC, between about 1 to about 9 times its CMC, between about 1 to about 8 times its CMC, between about 1 to about 7 times its CMC, between about 1 to about 6 times its CMC, between about 1 to about 5 times its CMC, between about 1 to about 4 times its CMC, between about 1 to about 3 times its CMC, or between about 1 to about 2 times its CMC. In some aspects, the DDM is present at a concentration between about 5 to about 10 times its CMC. In some aspects, the OG is present at a concentration which is at least about 0.1 times its CMC, at least about 0.2 times its CMC, at least about 0.3 times its CMC, at least about 0.4 times its CMC, at least about 0.5 times its CMC, at least about 0.6 times its CMC, at least about 0.7 times its CMC, at least about 0.8 times its CMC, at least about 0.9 times its CMC, or at least about 1 time its CMC. In some aspects, the OG is present at a concentration between about 0.1 to about 1 times its CMC. In some aspects, the OG is present at a concentration which is about 0.5 times its CMC, and DDM is present at a concentration which is between about 5 to about 10 times its CMC. In some aspects, (i) the OG is present at a concentration which is about 0.5 times its CMC, and the DDM is present at a concentration which is about 5 times its CMC, (ii) the OG is present at a concentration which is about 0.5 times its CMC, and the DDM is present at a concentration which is about 7.5 times its CMC, (iii) the OG is present at a concentration which is about 0.5 times its CMC, and the DDM is present at a concentration which is about 10 times its CMC, or (iv) the OG is present at a concentration which is about 0.75 times its CMC, and the DDM is present at a concentration which is about 5 times its CMC.

The present disclosure provides a method to treat a disease or condition comprising administering to a subject a therapeutic protein manufactured by a process comprising a viral inactivation step according to the methods disclosed herein, or a viral inactivation step comprising the use of the composition disclosed herein. Also provided is a pharmaceutical composition manufactured by a process comprising a viral inactivation step according to the methods disclosed herein, or a viral inactivation step comprising the use of the compositions disclosed herein. Also provided is a method of manufacture a therapeutic protein comprising a viral inactivation step according to the methods disclosed herein, or a viral inactivation step comprising the use of the compositions disclosed herein.

The present disclosure also provides a kit comprising a composition disclosed herein, and instructions for inactivating a virus, e.g., instructions for inactivating a virus according to the methods disclosed herein.

### BRIEF DESCRIPTION OF THE DRAWINGS/FIGURES

FIG. 1. (A) Schematic showing downstream unit operations involving virus inactivation (VI) and detergent clearance. (B) Strategy for screening conditions for VI involving stability screening, detergent clearance followed by viral clearance study. The stability study was divided into two segments. A first initial screening involved assessing aggregate formation for protein DS at concentrations comparable to process conditions representative of VI. The DS was treated with high and low concentrations of detergent at room temperature. The detergent conditions that showed levels of aggregate formation less than or comparable to control (no detergent) over a 24 hour period were carried over for a more comprehensive stability screening in alignment with the process developed. The protein was incubated at the highest temperature, i.e., room temperature and longest duration that conformed to the acceptable operating range as a worst case for stability. Quality attributes tested included aggregation, potency, charge variance, oxidation-deamidation and glycosylation. Following stability, the detergent and impurity clearance by the chromatographic step following VI is tested. The final step in the VI screening involved VI study with Bio Safety Level-2 (BSL2) viruses at a third party testing site.

FIG. 2. SEC (size exclusion chromatography) analyzing high molecular weight (HMW) formation in DS (drug substance) in (A) High protein concentration DS, (B) Low protein concentration DS, and (C) protein A eluate from detergent-spiked clarified cell culture harvest of two fusion proteins, Fus1 (abatacept) and Fus2 (belatacept). High molecular weight (HMW) formation was greatest for detergent OG with higher HMW formation for Fus1 in comparison to Fus2 as seen for low concentration DS. (D) Prediction profile for HMW formation based on statistical model where final HMW is a function of protein and detergent concentration as well as initial HMW content (R²=0.91, ANOVA p value=0.0002).

FIG. 3. SAP (Spatial-Aggregation-Propensity) model showing the difference in hydrophobicity arising from the main difference in the amino acid sequence of the fusion proteins Fus1 (abatacept) and Fus2 (belatacept). Two point mutations of in Fus2 with respect to Fus1, namely, Leu (L) to Glu (E) and Ala (A) to Tyr (Y) in the CTLA4 domain, showed that the mutations disrupted a big hydrophobic patch in Fus1 into a smaller hydrophobic patch in Fus2.

FIG. 4. (A-C) High concentration drug substance (DS) aggregation kinetics for detergents, OG, DDM and LDAO, for fusion proteins Fus1 and Fus2. (A) Aggregate formation over time. (B) Kinetics of aggregate formation. (C) Rate constant of HMW formation in relation to the HLB (Hydrophilic Lipophilic Balance) number of detergents. The greater the hydrophobicity of different detergents, the lower the HLB and greater the rate of HMW formation in both Fus1 and Fus2 DS. Thus the fastest rate of aggregation was observed for detergent, OG, followed by DDM and then LDAO. (D-E) Analysis of factors influencing rate constant of aggregation for fusion proteins, Fus1 and Fus2, in different protein matrices and detergents, OG and DDM. (D) Rate constant and protein concentration for the different protein-detergent systems. (E) Prediction profile for rate constant of HMW formation for data in (D). The statistical model showed the rate constant to be a function of protein and detergent concentration (R²=0.84, ANOVA p value=0.0002).

FIG. 5. (A) Protein A run on detergent-spiked Fusion protein 1 harvest. (B) Detergent clearance during Protein A run. Fus1 and Fus2 harvest was spiked with detergents, OG and DDM at 1×CMC (0.68 w/v %) and 10×CMC (0.061 w/v %) respectively. The harvest was then Protein A purified for 1 hour, and the flow through and eluate were collected and quantified for the respective detergent. Both detergents were below the limit of detection for Protein A eluate. (C-E) Impurity clearance during Protein A run for Fus1 harvest was spiked with detergents. The harvest was then Protein A purified at 1 hour, 8 hours, 24 hours, and 57 hours, the eluates were collected and quantified for (C) Deoxyribonucleic acid (DNA), (D) Host cell protein (HCP) and (E) residual Protein A. The results were averaged for the different time points and were comparable to the control (no detergent) and Triton X-100 spiked Fusion protein 1 harvest.

FIG. 6. Inactivation of virus. (A) X-MuLV in monoclonal antibody, mAb1. (B) X-MuLV, A-MuLV and HSV-1 in fusion protein, Fus1 harvest. Log reduction value (LRV) of virus in different therapeutic modalities was reported after a 60 minutes hold at 2-8° C.; LRV value of 4.0 was the minimum clearance necessary to consider the inactivation to be effective. Error bars represent assay variability. Greater than 4.0 LRV was observed for detergents OG, Zwittergent 3-12 and CG 110 at all concentrations. Virus inactivation was dependent on concentration for the detergents, DDM, LDAO and CG-650. These detergents showed greater than 4.0 LRV at 10×CMC (Critical Micelle Concentration) but less than 1.0 LRV at 1×CMC. The viable detergents from (A) were carried over for inactivation studies with viruses. (B) A-MuLV and HSV-1 in Fus1 harvest. HSV-1 shows greater susceptibility to inactivation than A-MuLV under all conditions. For both viruses, greater than 4.0 LRV was observed for detergents OG 1×CMC, ECOSURF™ 10×CMC and LDAO 10×CMC. Although DDM showed greater than 4.0 LRV at both 5× and 10×CMC for HSV-1, it only showed approximately 2.2 LRV for A-MuLV.

FIG. 7A. Kinetics of inactivation for virus X-MuLV in monoclonal antibody, mAb1. LRV of virus in different therapeutic modalities was reported over time at 2-8° C.

FIG. 7B. Kinetics of inactivation for virus A-MuLV. LRV of virus in different therapeutic modalities was reported over time at 2-8° C.

FIG. 7C. Kinetics of inactivation for virus HSV-1 in fusion protein, Fus1 harvest. LRV of virus in different therapeutic modalities was reported over time at 2-8° C.

FIG. 8. Product Quality Attributes for monoclonal antibody mAb1 with a control of untreated Hydrophobic Interaction Chromatography (HIC) pool. (A) Size exclusion based analysis of aggregate formation shows very little High Molecular Weight (HMW) species in detergent versus control samples. Caliper HT NR (High throughput non-reduced) samples show little Low Molecular Weight (LMW) species formation relative to control (B) Imaged capillary isoelectric focusing (iCIEF)-based charge distribution profile in different detergents shows little to no deviation from the control (C) Cell-based potency against mAb1-antigen.

FIG. 9. iCIEF-based profile (A) Acidic (B) Main (C) Basic for concentrated Fus1 drug substance (DS); post UF-DF DS, the acceptance criteria of Fus1 was main peak (Group 2)≥90%, the acidic peak (Group 1)≤3% and the basic one (Group 3)≥8. iCIEF profile (D) Acidic (E) Main (F) Basic for concentrated Fus2 DS; post UF-DF DS, the acceptance criteria of Fus2 involved a main peak (Group 2)≥79% while the acidic peak (Group 1)≤5% and the basic one (Group 3)≤20%.

FIG. 10. Oxidation and deamidation profiles for Fus1 and Fus2 Protein A eluates. Oxidation profiles of (A) Fus1 (B) Fus2, the acceptable range for oxidation for final drug substance being 1.3% for Fus1 and 2.9% for Fus2. Deamidation profiles of (C) Fus1 (D) Fus2—the acceptable range for deamidation for final drug substance being 5.9% for Fus1 and 8.5% for Fus2.

FIG. 11. B7 binding efficiency relative to reference material for fusion protein DS at high concentration in (A) Fus1 (B) Fus2 and at low concentration of 3 g/L in (C) Fus1 (D) Fus2. The acceptable limit for potency was 70 to 130% for Fus1 and 75 to 125% for Fus2. Higher concentration DS was worst case for potency particularly for OG at 10×CMC in Fus1, which showed less than 60% potency at 24 hour.

FIG. 12. Sialic acid content for Fus1 and Fus2 Protein A eluates. NGNA (N-glycolylneuraminic acid): protein molar concentration in (A) Fus1 (B) Fus2, the lower values being safe for administration to patients. NANA (N-acetylneuraminic acid): protein molar concentration in (C) Fus1 (D) Fus2 were within acceptable range of 8 to 12% and 6 to 8.5% respectively.

FIG. 13. HMW species in Protein A eluate of fusion protein, Fus1 harvest spiked with detergent, OG. HMW content for (A) control or no detergent and (B) OG samples was analyzed with SEC (size exclusion chromatography).

FIG. 14. SEC-analyzed aggregate formation in DS and clarified cell culture harvest of fusion proteins Fus1 (A) and (B) and Fus2 (C) and (D), respectively. Fus1 DS was at 39.5 to 45.2 g/L (A) and Fus2 DS was at 21.2 to 24.3 g/L (C). HMW formation (colored bars) was greatest for detergent OG, followed by DDM and LDAO with higher HMW formation for Fus1 in comparison to Fus2.

FIG. 15. Dependence of aggregate formation on the detergent and protein concentration and buffer matrix for fusion proteins, Fus1 in panels (A) OG (B) DDM and Fus2 in panels (C) OG (D) DDM. The aggregate formation showed first order kinetics with greatest rate constant for OG followed by DDM for Fus1 in panels (E) and (F) and Fus2 in panels (G) and (H), respectively.

FIG. 16 shows the chemical structures, names, and abbreviations of detergents presented in the disclosure.

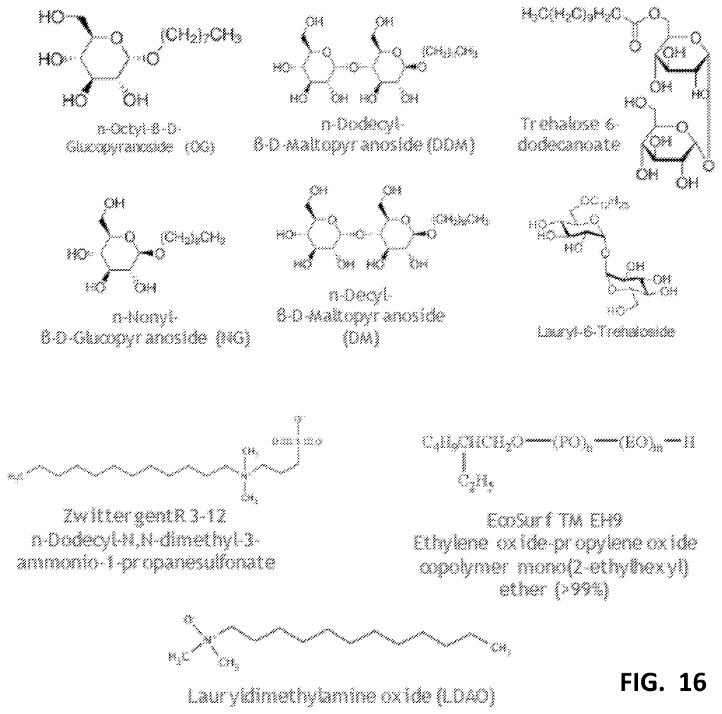

FIG. 17 shows the chemical structures of detergents and combinations thereof used in the present disclosure, and the concentrations used. Concentrations are fold-numbers (e.g., 5× means 5-fold) with respect to their respective CMC values.

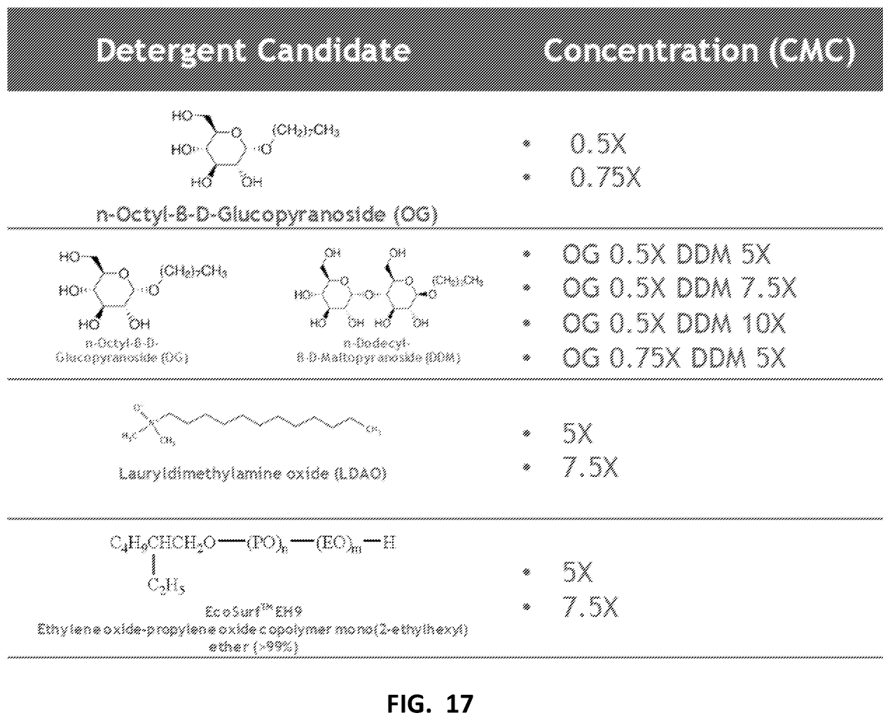

FIG. 18 is a schematic representation and table describing the properties of the model viruses used in the experiments of the present disclosure.

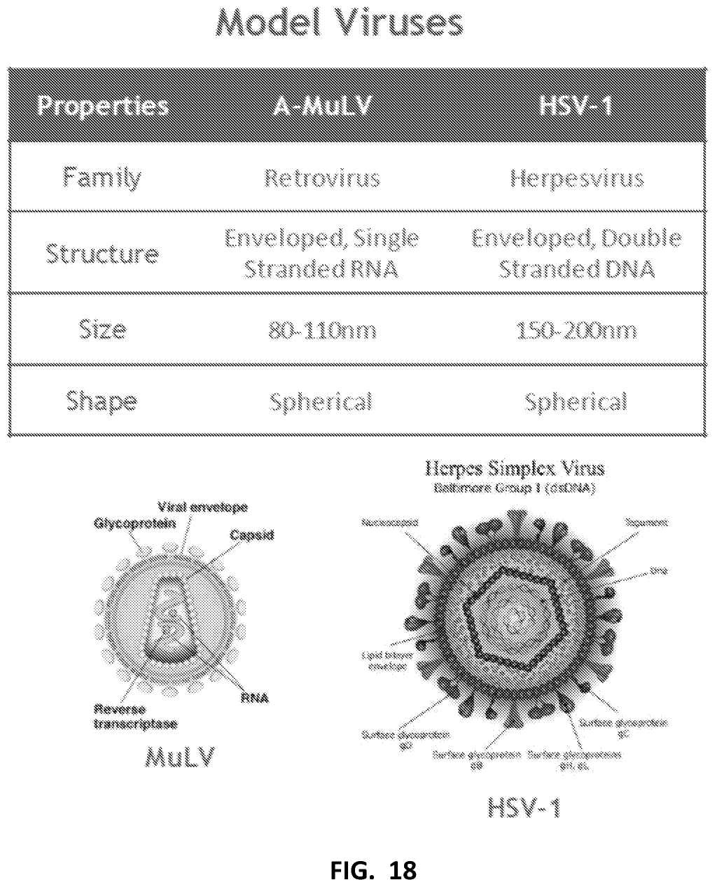

FIG. 19 shows the viral inactivation effectiveness of different detergents with A-MuLV and HSV-1 at an incubation time of 60-minutes. The data is presented as an average of two replicates and the error bar is the standard deviation.

FIG. 20 shows the viral inactivation effectiveness of the OG 0.5×DDM 5× detergent combination at different time points (0, 15, 30, and 60 minutes). The data is presented as an average of two replicates and the error bar is the standard deviation.

FIG. 21 shows the effects of different detergents on protein aggregation formation in the Protein A pool. The data is presented as an average of three replicates and the error bar is the standard deviation

FIG. 22 shows the effects of different detergents on the sialic acid content of the Protein A pool. The data is presented as an average of three replicates and the error bar is the standard deviation.

FIG. 23 shows the effects of different detergents on protein stability profile. The data is presented as an average of three replicates and the error bar is the standard deviation.

FIG. 24 shows the effects of different detergents on the impurity profile of the Protein A pool. The data is presented as an average of three replicates and the error bar is the standard deviation.

### DETAILED DESCRIPTION

The present disclosure relates to detergent-mediated inactivation of lipid-enveloped viruses. In some aspects, the disclosure relates to detergent-mediated viral inactivation for the abatacept purification process approved by EMA (European Medicines Agency) to manufacture ORENCIA®. The current commercially available detergent for such process is Triton X-100.

Triton X-100 is a nonionic surfactant that has a hydrophilic polyethylene oxide chain and an aromatic hydrocarbon group 1 4-(1,1,3,3-tetramethylbutyl) phenol group. Triton X-100 has been used extensively in the pharmaceutical industry for inactivating viruses. However, through stepwise removal of ethylene oxide, Triton X-100 degrades into 4-tert-octylphenol, which is an endocrine disruptor with adverse estrogenic effect on aquatic species, animals, and humans. This alkyl phenol is listed as Substance of Very High Concern (SHVC) and published in EU Annex XIV by European Chemicals Agency (ECHA) under the REACh1 regulation. As such ECHA has mandated a sunset date of 2021 to replace Triton X-100 with eco-friendly detergents in all manufacturing processes.

Several environmentally friendly detergents for the viral inactivation step of biologics manufacturing processes have been identified in the art, e.g., Lauryldimethylamine Oxide (LDAO) and ECOSURF™ EH9. These systems for viral inactivation use a single detergent. The present disclosure provides a detergent mixture comprising two environmentally sustainable detergents: n-Octyl-β-D-Glucopyranoside (OG) and n-Dodecyl-β-D-Maltopyranoside (DDM). The present disclosure shows that the performance of this detergent combination is superior to that of Lauryldimethylamine Oxide (LDAO), ECOSURF™ EH9, or Triton X-100 in the purification of therapeutic proteins such as abatacept and belatacept. Surprisingly, it was found that while OG concentrations below its critical micelle concentration (CMC), e.g., 0.5×CMC, were insufficient for viral inactivation (defined as a LRV equal or above 4), combinations of sub-CMC amounts of OG with DDM (e.g., in the 5× to 10×CMC range) were highly effective for viral inactivation, while having no impact on protein stability, protein charge distribution (e.g., sialic acid levels), impurity clearance, protein deamination, or protein oxidation. Accordingly, the disclosed combination of OG and DDM can be used as substitute of Triton X-100 for the viral inactivation step in the production of biologics, for example, in the process used to manufacture ORENCIA®.

### Terms

In order that the present disclosure can be more readily understood, certain terms are first defined. As used in this application, except as otherwise expressly provided herein, each of the following terms shall have the meaning set forth below. Additional definitions are set forth throughout the application.

The singular forms “a”, “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

It is understood that wherever aspects are described herein with the language “comprising,” otherwise analogous aspects described in terms of “consisting of” and/or “consisting essentially of” are also provided.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As described herein, any concentration range, percentage range, ratio range or integer range is to be understood to include the value of any integer within the recited range and, when appropriate, fractions thereof (such as one tenth and one hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. The headings provided herein are not limitations of the various aspects of the disclosure, which can be had by reference to the specification as a whole. Accordingly, the terms defined are more fully defined by reference to the specification in its entirety.

Abbreviations used herein are defined throughout the present disclosure. Various aspects of the disclosure are described in further detail in the following subsections.

Amino acids are referred to herein by either their commonly known three letter symbols or by the one-letter symbols recommended by the IUPAC-IUB Biochemical Nomenclature Commission. Unless otherwise indicated, amino acid sequences are written left to right in amino to carboxy orientation.

The methods disclosed herein can be used, e.g., for the production of a biological such as an antibody or a fusion protein. As use herein, the term “antibody” (Ab) shall include, without limitation, a glycoprotein immunoglobulin that binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H}1, C_{H}2 and C_{H}3. Each light chain comprises a light chain variable region (abbreviated herein as V_{L}) and a light chain constant region. The light chain constant region is comprises one constant domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

The term “chromatography” refers to any kind of technique which separates a protein of interest (e.g., an antibody or a fusion protein such as abatacept or belatacept) from other molecules (e.g., contaminants) present in a mixture, in which the protein of interest is separated from other molecules (e.g., contaminants) as a result of differences in rates at which the individual molecules of the mixture migrate through a stationary medium under the influence of a moving phase, or in bind and elute processes.

The term “chromatography column” or “column” in connection with chromatography as used herein, refers to a container, frequently in the form of a cylinder or a hollow pillar which is filled with the chromatography medium or resin. The chromatography medium or resin is the material that provides the physical and/or chemical properties that are employed for purification. The term “chromatography medium” or “chromatography matrix” are used interchangeably herein and refer to any kind of sorbent, resin or solid phase that in a separation process separates a protein of interest (e.g., an Fc region containing protein such as an immunoglobulin) from other molecules present in a mixture. Non-limiting examples include particulate, monolithic or fibrous resins as well as membranes that can be put in columns or cartridges. Examples of materials for forming the matrix include polysaccharides (such as agarose and cellulose); and other mechanically stable matrices such as silica (e.g. controlled pore glass), poly(styrenedivinyl)benzene, polyacrylamide, ceramic particles and derivatives of any of the above.

A “chromatography ligand” is a functional group that is attached to the chromatography medium and that determines the binding properties of the medium. Examples of “ligands” include, but are not limited to, ion exchange groups, hydrophobic interaction groups, hydrophilic interaction groups, thiophilic interactions groups, metal affinity groups, affinity groups, bioaffinity groups, and mixed mode groups (combinations of the aforementioned). In some aspects, a chromatography ligand can be Protein A.

As used herein, the term “affinity chromatography” refers to a protein separation technique in which a protein of interest (e.g., an antibody) is specifically bound to a ligand that is specific for the therapeutic protein of interest. Such a ligand is generally referred to as a biospecific ligand. In some aspects, the biospecific ligand (e.g., Protein A or a functional variant thereof) is covalently attached to a chromatography medium and is accessible to the therapeutic protein of interest in solution as the solution contacts the chromatography medium.

The therapeutic protein of interest generally retains its specific binding affinity for the biospecific ligand during the chromatographic steps, while other solutes and/or proteins in the mixture do not bind appreciably or specifically to the ligand. Binding of the therapeutic protein of interest to the immobilized ligand allows contaminating proteins or protein impurities to be passed through the chromatography matrix while the therapeutic protein of interest remains specifically bound to the immobilized ligand on the solid phase material. The specifically bound therapeutic protein of interest is then removed in active form from the immobilized ligand under suitable conditions (e.g., low pH, high pH, high salt, competing ligand etc.), and passed through the chromatographic column with the elution buffer, free of the contaminating proteins or protein impurities that were earlier allowed to pass through the column.

Any component can be used as a ligand for purifying its respective specific binding protein, e.g., antibody. However, in various methods according to the present disclosure, Protein A is used as a ligand for an Fc region in a target protein (e.g., abatacept or belatacept). The conditions for elution from the biospecific ligand (e.g., Protein A) of the target protein (e.g., an Fc region containing protein such as abatacept or belatacept) can be readily determined by one of ordinary skill in the art.

In some aspects, Protein G or Protein L or a functional variant thereof can be used as a biospecific ligand. In some aspects, a biospecific ligand such as Protein A is used at a pH range of 5-9 for binding to an Fc region containing protein, washing or re-equilibrating the biospecific ligand/target protein conjugate, followed by elution with a buffer having pH about or below 4 which contains at least one salt.

The term “aggregation” refers to the tendency of a polypeptide, e.g., an antibody or a fusion protein (e.g., abatacept or belatacept), to form complexes with other molecules (such as other molecules of the same polypeptide) thereby forming high molecular weight (HMW) aggregates. Exemplary methods of measuring the formation of aggregates include analytical size exclusion chromatography as described in the Examples herein. Relative amounts of aggregation may be determined with respect to a reference compound, e.g., to identify a polypeptide having reduced aggregation. Relative amounts of aggregation can also be determined with respect to a reference formulation.

As used herein the terms “HMW” refers to any one or more unwanted proteins present in a mixture with a molecular weight generally higher than that of the desired protein of interest, e.g., an antibody or fusion protein such as abatacept or belatacept. High molecular weight proteins can include dimers, trimers, tetramers, or other multimers. These proteins can be either covalently or non-covalently linked, and can also, for example, consist of misfolded monomers in which hydrophobic amino acid residues are exposed to a polar solvent, and can cause aggregation.

The term “detergent” refers to an agent or combination thereof that may comprise salts of long-chain aliphatic bases or acids, or hydrophilic moieties such as sugars, and that possess both hydrophilic and hydrophobic properties. Having both hydrophilic and hydrophobic properties, the detergent can exert particular effects. As used herein, detergents have the ability to disrupt viral envelopes and inactivate viruses.

As used herein, an “eco-friendly” or “environmentally compatible” substance is a substance that causes minimal harmful effects on the environment. For example, an environmentally compatible substance would essentially be nontoxic to animal and/or plant life. Methods to determine whether a detergent is environmentally compatible under the conditions of the manufacturing processes of present disclosure are known in the art. For example, the Organisation for Economic Co-operation and Development provides guidelines for testing chemical safety. These guidelines can be found, for example, on the world wide web at oecd.org/chemicalsafety/testing/oecdguidelinesforthetestingofchemicals.htm. Examples of tests that can be used to determine whether a detergent is eco-friendly comprise, e.g., the Daphnia magna reproduction test (OECD 211), the Freshwater Alga and Cyanobacteria, Growth Inhibition Test (OECD 201), the Daphnia sp., Acute Immobilization Assay (OECD 202), the Fish, Acute Toxicity Test (OECD 203), the Activated Sludge, Respiration Inhibition Test (Carbon and Ammonium Oxidation) (OECD 209), and the Ready Biodegradability test (OECD 301).

A “predicted environmental concentration” or “PEC” is the predicted concentration of a substance (e.g., a detergent) in waste material discharged into the receiving water body in environment. For example, a predicted environmental concentration of a detergent used for viral inactivation in the preparation of a therapeutic protein is the concentration of detergent in the waste stream that is discharged into the environment.

A “predicted no-effect concentration” or “PNEC” is the predicted concentration of a substance (e.g., a detergent) in waste material that is safe for discharging to the environment without harmful effects; for example, to the biota of the receiving fresh water and/or marine water.

A “product feedstream” or alternatively, “feedstream” is the material or solution provided for a process purification method which contains a therapeutic protein of interest (e.g., abatacept or belatacept) and which may also contain various impurities. Non-limiting examples may include, for example, harvested cell culture fluid (HCCF), or the collected pool containing the therapeutic protein of interest after one or more purification process steps. It is understood that the uses of the compositions and methods of the present disclosure are not limited to manufacture feedstreams of biological, and can be used to inactivate enveloped virus in any solution or medium in which viral inactivation is desired. In some aspects, viral inactivation can be conduct in a solution. In some aspects, viral inactivation can be conducted on a solid surface. In some aspects, viral inactivation can be conducted on the surface of the body of a mammal. In some aspects, the product feedstream comprises a harvest (e.g., a harvested cell cell culture fluid), a load (e.g., a loading solution of a chromatography or filtration, i.e., a chromatography load or a filtration load), an eluate (e.g., a chromatography eluate), a filtrate (e.g., a solution collected after a solution has been filtered), or any combination thereof. In general, the methods disclosed herein can be used for viral inactivation in any solution comprising a virus or suspected to contain a virus.

“Impurities” refer to materials that are different from the desired polypeptide product. The impurity includes, without limitation: host cell materials, such as host cell protein (HCP); leached Protein A; nucleic acid; a variant, size variant, fragment, aggregate or derivative of the desired polypeptide; another polypeptide; endotoxin; viral contaminant; cell culture media component, etc.

As used herein, the terms “inactivating a virus” or “virus inactivation” refer to a process where a virus can no longer infect cells, replicate, and propagate, and per se virus removal. As such, the term “virus inactivation” refers generally to the process of making a fluid disclosed herein completely free of infective viral contaminants. Any degree of viral inactivation using the methods disclosed herein is desirable. However, it is desirable to achieve the degree of viral inactivation necessary to meet strict safety guidelines for pharmaceuticals. These guidelines are set forth by the WHO and well known to those of skill in the art.

### I. Eco-Friendly Detergent Compositions and Method for Viral Inactivation

The present disclosure provides compositions and methods of inactivating a virus in a product feedstream (e.g., a harvest, load, eluate, or filtrate) in a manufacturing process of a therapeutic protein using environmentally compatible detergents. In some aspects, the methods disclosed herein comprise contacting the product feedstream with n-Dodecyl-β-D-Maltopyranoside (DDM) (CAS #69227-93-6), alone or in combination with n-Octyl-β-D-Glucopyranoside (OG) (CAS #29836-26-8). Thus, in some aspects, the present disclosure provides compositions and methods of inactivating a virus in a product feedstream (e.g., a harvest, load, eluate, or filtrate) in a manufacturing process of a therapeutic protein, e.g., a biologic. In some aspects, the methods of inactivating a virus disclosed herein comprise contacting a virus, e.g., a virus in a feed stream, with a composition comprising DDM and OG at a range of concentrations disclosed herein.

The use of the environmentally compatible detergents disclosed herein does not adversely affect product quality of the therapeutic protein while effectively inactivating viral contaminants in the feedstream (e.g., a harvest, load, eluate, or filtrate). For example, the use of environmentally compatible detergents of the present disclosure does not result in an increase in product variants such as, but not limited to, size variants including product fragments and aggregates, charge variants including acidic and basic variants, deamination variants, oxidation variants, and glycosylation variants.

Generally, the manufacturing process for a therapeutic protein includes expression of the protein in a host cell. In some aspects, the host cells are lysed to release the therapeutic protein. In other examples, the therapeutic protein is secreted in the media. The harvested cell culture fluid comprising the desired therapeutic protein can be clarified and subject to one or more chromatographies to purify the desired therapeutic protein from impurities. Chromatography may include flow-through chromatography where the desired product flows through the chromatography and impurities are retained by the chromatography, and/or bind and elute chromatography, where the desired product, i.e., the therapeutic protein, is retained by the chromatography medium, and impurities flow through the chromatography.

Methods of viral inactivation using an environmentally compatible detergent combination disclosed herein can be performed at any step during the manufacturing process of a biologic. In some aspects, the virus is inactivated by contacting the harvested cell culture fluid (HCCF) with an environmentally compatible detergent combination disclosed herein. In other aspects, the virus is inactivated by contacting a capture pool or a recovered product pool with an environmentally compatible detergent combination disclosed herein. A capture pool and/or a recovered product pool is the partition of a feedstream comprising the desired product i.e., the therapeutic protein, in a separation step in the purification of the product, such as a chromatography, a centrifugation, a filtration, and the like.

In other aspects, the virus is inactivated by contacting a product feedstream with an environmentally compatible detergent combination disclosed herein before subjecting the feedstream to a virus filtration step. In other aspects, the virus is inactivated by contacting a product feedstream with an environmentally compatible detergent combination disclosed herein after subjecting the feedstream to a virus filtration step.

The present disclosure provides methods of inactivating virus in a product feedstream (e.g., a harvest, load, eluate, or filtrate) in a manufacturing process of a therapeutic protein using environmentally compatible detergents combination disclosed herein wherein the product quality of the therapeutic protein is maintained during the process, while viral contaminants are effectively inactivated. In some aspects, treatment of the feedstream (e.g., a harvest, load, eluate, or filtrate) in a manufacturing process of a therapeutic protein with an environmentally compatible detergent combination disclosed herein to inactivate virus in the feedstream does not increase the amount of product variants in the manufacturing process; for example, compared to the manufacturing process without detergent or with Triton X-100.

In general, the viral inactivation methods disclosed herein comprise using a detergent composition having a combination of glycosides, e.g., a maltoside and a glucoside, such as a maltopyranoside and a glucopyranoside. In some aspects, the maltoside is an alkyl maltoside. In some aspects, the glucoside is an alkyl glucoside.

In some aspects, the maltoside comprises n-decyl-β-D-maltopyranoside (DM). In some aspects, the maltoside comprises n-Dodecyl-β-D-maltopyranoside (DDM). In some aspects, the maltoside comprises 6-Cyclohexyl-1-hexyl-β-D-maltopyranoside (Cymal-6). In some aspects, the maltoside is selected from the group consisting of n-decyl-β-D-maltopyranoside (DM), n-Dodecyl-β-D-maltopyranoside (DDM), 6-Cyclohexyl-1-hexyl-β-D-maltopyranoside (Cymal-6), and combinations thereof. In some aspects, the glucoside comprises n-Octyl-β-D-Glucopyranoside (OG).

In some aspects, the detergent combination comprises a first glycoside which is n-Dodecyl-β-D-maltopyranoside (DDM)

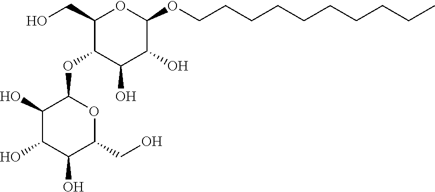 [SMILES: CCCCCCCCCCO[C@@H]1O[C@H](CO)[C@@H](O[C@H]2O[C@H](CO)[C@@H](O)[C@H](O)[C@H]2O)[C@H](O)[C@H]1O]

and a second glycoside which is β-D-Octyl glucoside (OG)

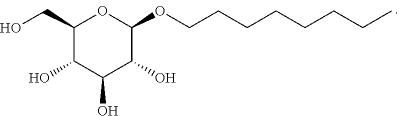 [SMILES: CCCCCCCCO[C@@H]1O[C@H](CO)[C@@H](O)[C@H](O)[C@H]1O]

In some aspects, the detergent combination of the present disclosure comprises DDM present at a concentration which is at least about 1 time (1×), at least about 2 times (2×), at least about 3 times (3×), at least about 4 times (4×), at least about 5 times (5×), at least about 6 times (6×), at least about 7 times (7×), at least about 7.5 times (7.5×), at least about 8 times (8×), at least about 9 times (9×), at least about 10 times (10×), at least about 11 times (11×), at least about 12 times (12×), at least about 13 times (13×), at least about 14 times (14×), at least about 15 times (15×), at least about 16 times (16×), at least about 17 times (17×), at least about 18 times (18×), at least about 19 times (19×), or at least about 20 times (20×) times its critical micelle concentration (CMC). In some aspects, the detergent combination of the present disclosure comprises DDM present at a concentration which is about 1 time (1×), about 2 times (2×), about 3 times (3×), about 4 times (4×), about 5 times (5×), about 6 times (6×), about 7 times (7×), about 7.5 times (7.5×), about 8 times (8×), about 9 times (9×), about 10 times (10×), about 11 times (11×), about 12 times (12×), about 13 times (13×), about 14 times (14×), about 15 times (15×), about 16 times (16×), about 17 times (17×), about 18 times (18×), about 19 times (19×), or about 20 times (20×) times its CMC.

The CMC of DDM is 0.0061% w/v. Thus, a 1×CMC concentration of DDM is 0.0061% w/v, a 2×CMC concentration of DDM is 0.0122% (w/v), a 3×CMC concentration of DDM is 0.0183% (w/v), a 4×CMC concentration of DDM is 0.0244% (w/v), a 5×CMC concentration of DDM is 0.0305% (w/v), a 6×CMC concentration of DDM is 0.0366% (w/v), a 7×CMC concentration of DDM is 0.0427% (w/v), a 7.5×CMC concentration of DDM is 0.04575% (w/v), a 8×CMC concentration of DDM is 0.0488% (w/v), a 9×CMC concentration of DDM is 0.0549% (w/v), a 10×CMC concentration of DDM is 0.061% (w/v), a 11×CMC concentration of DDM is 0.0671% (w/v), a 12×CMC concentration of DDM is 0.0732% (w/v), a 13×CMC concentration of DDM is 0.0793% (w/v), a 14×CMC concentration of DDM is 0.0854% (w/v), a 15×CMC concentration of DDM is 0.0915% (w/v), a 16×CMC concentration of DDM is 0.0976% (w/v), a 17×CMC concentration of DDM is 0.1037% (w/v), a 18×CMC concentration of DDM is 0.1098% (w/v), a 19×CMC concentration of DDM is 0.1159% (w/v), and a 20×CMC concentration of DDM is 0.122 (w/v). The molecular weight of DDM is 510.6 g/mol. Thus, a person of ordinary skill in the art could easily convert the disclosed % (w/v) concentrations to molar concentrations.

In some aspects, the detergent combination of the present disclosure comprises DDM present at a concentration which is at least about 0.0061% (w/v), at least about 0.0122% (w/v), at least about 0.0183% (w/v), at least about 0.0244% (w/v), at least about 0.0305% (w/v), at least about 0.0366% (w/v), at least about 0.0427% (w/v), at least about 0.04575% (w/v), at least about 0.0488% (w/v), at least about 0.0549% (w/v), at least about 0.061% (w/v), at least about 0.0671% (w/v), at least about 0.0732% (w/v), at least about 0.0793% (w/v), at least about 0.0854% (w/v), at least about 0.0915% (w/v), at least about 0.0976% (w/v), at least about 0.1037% (w/v), at least about 0.1098% (w/v), at least about 0.1159% (w/v), or at least about 0.122% (w/v).

In some aspects, the DDM is present at a concentration which is between about 1 time (1×) to about 20 times (20×) its CMC, between about 1 time (1×) to about 19 times (19×) its CMC, between about 1 time (1×) to about 18 times (18×) its CMC, between about 1 time (1×) to about 17 times (17×) its CMC, between about 1 time (1×) to about 16 times (16×) its CMC, between about 1 time (1×) to about 15 times (15×) its CMC, between about 1 time (1×) to about 14 times (14×) its CMC, between about 1 time (1×) to about 13 times (13×) its CMC, between about 1 time (1×) to about 12 times (12×) its CMC, between about 1 time (1×) to about 11 times (11×) its CMC, between about 1 time (1×) to about 10 times (10×) its CMC, between about 1 time (1×) to about 9 times (9×) its CMC, between about 1 time (1×) to about 8 times (8×) its CMC, between about 1 time (1×) to about 7 times (7×) its CMC, between about 1 time (1×) to about 6 times (6×) its CMC, between about 1 time (1×) to about 5 times (5×) its CMC, between about 1 time (1×) to about 4 times (4×) its CMC, between about 1 time (1×) to about 3 times (3×) its CMC, or between about 1 time (1×) to about 2 times (2×) its CMC.

In some aspects, the DDM is present at a concentration which is between about 2 times (2×) to about 20 times (20×) its CMC, between about 2 times (2×) to about 19 times (19×) its CMC, between about 2 times (2×) to about 18 times (18×) its CMC, between about 2 times (2×) to about 17 times (17×) its CMC, between about 2 times (2×) to about 16 times (16×) its CMC, between about 2 times (2×) to about 15 times (15×) its CMC, between about 2 times (2×) to about 14 times (14×) its CMC, between about 2 times (2×) to about 13 times (13×) its CMC, between about 2 times (2×) to about 12 times (12×) its CMC, between about 2 times (2×) to about 11 times (11×) its CMC, between about 2 times (2×) to about 10 times (10×) its CMC, between about 2 times (2×) to about 9 times (9×) its CMC, between about 2 times (2×) to about 8 times (8×) its CMC, between about 2 times (2×) to about 7 times (7×) its CMC, between about 2 times (2×) to about 6 times (6×) its CMC, between about 2 times (2×) to about 5 times (5×) its CMC, between about 2 times (2×) to about 4 times (4×) its CMC, or between about 2 times (2×) to about 3 times (3×) its CMC.

In some aspects, the DDM is present at a concentration which is between about 3 times (3×) to about 20 times (20×) its CMC, between about 3 times (3×) to about 19 times (19×) its CMC, between about 3 times (3×) to about 18 times (18×) its CMC, between about 3 times (3×) to about 17 times (17×) its CMC, between about 3 times (3×) to about 16 times (16×) its CMC, between about 3 times (3×) to about 15 times (15×) its CMC, between about 3 times (3×) to about 14 times (14×) its CMC, between about 3 times (3×) to about 13 times (13×) its CMC, between about 3 times (3×) to about 12 times (12×) its CMC, between about 3 times (3×) to about 11 times (11×) its CMC, between about 3 times (3×) to about 10 times (10×) its CMC, between about 3 times (3×) to about 9 times (9×) its CMC, between about 3 times (3×) to about 8 times (8×) its CMC, between about 3 times (3×) to about 7 times (7×) its CMC, between about 3 times (3×) to about 6 times (6×) its CMC, between about 3 times (3×) to about 5 times (5×) its CMC, or between about 3 times (3×) to about 4 times (4×) its CMC.

In some aspects, the DDM is present at a concentration which is between about 4 times (4×) to about 20 times (20×) its CMC, between about 4 times (4×) to about 19 times (19×) its CMC, between about 4 times (4×) to about 18 times (18×) its CMC, between about 4 times (4×) to about 17 times (17×) its CMC, between about 4 times (4×) to about 16 times (16×) its CMC, between about 4 times (4×) to about 15 times (15×) its CMC, between about 4 times (4×) to about 14 times (14×) its CMC, between about 4 times (4×) to about 13 times (13×) its CMC, between about 4 times (4×) to about 12 times (12×) its CMC, between about 4 times (4×) to about 11 times (11×) its CMC, between about 4 times (4×) to about 10 times (10×) its CMC, between about 4 times (4×) to about 9 times (9×) its CMC, between about 4 times (4×) to about 8 times (8×) its CMC, between about 4 times (4×) to about 7 times (7×) its CMC, between about 4 times (4×) to about 6 times (6×) its CMC, or between about 4 times (4×) to about 5 times (5×) its CMC.

In some aspects, the DDM is present at a concentration which is between about 5 times (5×) to about 20 times (20×) its CMC, between about 5 times (5×) to about 19 times (19×) its CMC, between about 5 times (5×) to about 18 times (18×) its CMC, between about 5 times (5×) to about 17 times (17×) its CMC, between about 5 times (5×) to about 16 times (16×) its CMC, between about 5 times (5×) to about 15 times (15×) its CMC, between about 5 times (5×) to about 14 times (14×) its CMC, between about 5 times (5×) to about 13 times (13×) its CMC, between about 5 times (5×) to about 12 times (12×) its CMC, between about 5 times (5×) to about 11 times (11×) its CMC, between about 5 times (5×) to about 10 times (10×) its CMC, between about 5 times (5×) to about 9 times (9×) its CMC, between about 5 times (5×) to about 8 times (8×) its CMC, between about 5 times (5×) to about 7 times (7×) its CMC, or between about 5 times (5×) to about 6 times (6×) its CMC.

In some aspects, the DDM is present at a concentration which is between about 6 times (6×) to about 20 times (20×) its CMC, between about 6 times (6×) to about times 19 (19×) its CMC, between about 6 times (6×) to about 18 times (18×) its CMC, between about 6 times (6×) to about 17 times (17×) its CMC, between about 6 times (6×) to about 16 times (16×) its CMC, between about 6 times (6×) to about 15 times (15×) its CMC, between about 6 times (6×) to about 14 times (14×) its CMC, between about 6 times (6×) to about 13 times (13×) its CMC, between about 6 times (6×) to about 12 times (12×) its CMC, between about 6 times (6×) to about 11 times (11×) its CMC, between about 6 times (6×) to about 10 times (10×) its CMC, between about 6 times (6×) to about 9 times (9×) its CMC, between about 6 times (6×) to about 8 times (8×) its CMC, or between about 6 times (6×) to about 7 times (7×) its CMC.

In some aspects, the DDM is present at a concentration which is between about 7 times (7×) to about 20 times (20×) its CMC, between about 7 times (7×) to about 19 times (19×) its CMC, between about 7 times (7×) to about 18 times (18×) its CMC, between about 7 times (7×) to about 17 times (17×) its CMC, between about 7 times (7×) to about 16 times (16×) its CMC, between about 7 times (7×) to about 15 times (15×) its CMC, between about 7 times (7×) to about 14 times (14×) its CMC, between about 7 times (7×) to about 13 times (13×) its CMC, between about 7 times (7×) to about 12 times (12×) its CMC, between about 7 times (7×) to about 11 times (11×) its CMC, between about 7 times (7×) to about 10 times (10×) its CMC, between about 7 times (7×) to about 9 times (9×) its CMC, or between about 7 times (7×) to about 8 times (8×) its CMC.

In some aspects, the DDM is present at a concentration which is between about 8 times (8×) to about 20 times (20×) its CMC, between about 8 times (8×) to about 19 times (19×) its CMC, between about 8 times (8×) to about 18 times (18×) its CMC, between about 8 times (8×) to about 17 times (17×) its CMC, between about 8 times (8×) to about 16 times (16×) its CMC, between about 8 times (8×) to about 15 times (15×) its CMC, between about 8 times (8×) to about 14 times (14×) its CMC, between about 8 times (8×) to about 13 times (13×) its CMC, between about 8 times (8×) to about 12 times (12×) its CMC, between about 8 times (8×) to about 11 times (11×) its CMC, between about 8 times (8×) to about 10 times (10×) its CMC, or between about 8 times (8×) to about 9 times (9×) its CMC.

In some aspects, the DDM is present at a concentration which is between about 9 times (9×) to about 20 times (20×) its CMC, between about 9 times (9×) to about 19 times (19×) its CMC, between about 9 times (9×) to about 18 times (18×) its CMC, between about 9 times (9×) to about 17 times (17×) its CMC, between about 9 times (9×) to about times 16 (16×) its CMC, between about 9 times (9×) to about 15 times (15×) its CMC, between about 9 times (9×) to about 14 times (14×) its CMC, between about 9 times (9×) to about 13 times (13×) its CMC, between about 9 times (9×) to about 12 times (12×) its CMC, between about 9 times (9×) to about 11 times (11×) its CMC, or between about 9 times (9×) to about 10 times (10×) its CMC.

In some aspects, the DDM is present at a concentration which is between about 10 times (10×) to about 20 times (20×) its CMC, between about 10 times (10×) to about 19 times (19×) its CMC, between about 10 times (10×) to about 18 times (18×) its CMC, between about 10 times (10×) to about 17 times (17×) its CMC, between about 10 times (10×) to about times 16 (16×) its CMC, between about times 10 (10×) to about 15 times (15×) its CMC, between about times 10 (10×) to about 14 times (14×) its CMC, between about 10 times (10×) to about 13 times (13×) its CMC, between about 10 times (10×) to about 12 times (12×) its CMC, or between about 10 times (10×) to about 11 times (11×) its CMC.

In some aspects, the DDM is present at a concentration which is between about 11 times (11×) to about 20 times (20×) its CMC, between about 11 times (11×) to about 19 times (19×) its CMC, between about 11 times (11×) to about 18 times (18×) its CMC, between about 11 times (11×) to about 17 times (17×) its CMC, between about 11 times (11×) to about 16 times (16×) its CMC, between about 11 times (11×) to about 15 times (15×) its CMC, between about 11 times (11×) to about 14 times (14×) its CMC, between about 11 times (11×) to about 13 times (13×) its CMC, or between about 11 times (11×) to about 12 times (12×) its CMC.

In some aspects, the DDM is present at a concentration which is between about 12 times (12×) to about 20 times (20×) its CMC, between about 12 times (12×) to about 19 times (19×) its CMC, between about 12 times (12×) to about 18 times (18×) its CMC, between about 12 times (12×) to about 17 times (17×) its CMC, between about 12 times (12×) to about 16 times (16×) its CMC, between about 12 times (12×) to about 15 times (15×) its CMC, between about 12 times (12×) to about 14 times (14×) its CMC, or between about 12 times (12×) to about 13 times (13×) its CMC.

In some aspects, the DDM is present at a concentration which is between about 13 times (13×) to about 20 times (20×) its CMC, between about 13 times (13×) to about 19 times (19×) its CMC, between about 13 times (13×) to about 18 times (18×) its CMC, between about 13 times (13×) to about 17 times (17×) its CMC, between about 13 times (13×) to about 16 times (16×) its CMC, between about 13 times (13×) to about 15 times (15×) its CMC, or between about 13 times (13×) to about 14 times (14×) its CMC.

In some aspects, the DDM is present at a concentration which is between about 14 times (14×) to about 20 times (20×) its CMC, between about 14 times (14×) to about 19 times (19×) its CMC, between about 14 times (14×) to about 18 times (18×) its CMC, between about 14 times (14×) to about 17 times (17×) its CMC, between about 14 times (14×) to about 16 times (16×) its CMC, or between about 14 times (14×) to about 15 times (15×) its CMC.

In some aspects, the DDM is present at a concentration which is between about 15 times (15×) to about 20 times (20×) its CMC, between about 15 times (15×) to about 19 times (19×) its CMC, between about 15 times (15×) to about 18 times (18×) its CMC, between about 15 times (15×) to about 17 times (17×) its CMC, or between about 15 times (15×) to about 16 times (16×) its CMC.

In some aspects, the DDM is present at a concentration which is between about 16 times (16×) to about 20 times (20×) its CMC, between about 16 times (16×) to about 19 times (19×) its CMC, between about 16 times (16×) to about 18 times (18×) its CMC, or between about 16 times (16×) to about 17 times (17×) its CMC.

In some aspects, the DDM is present at a concentration which is between about 17 times (17×) to about 20 times (20×) its CMC, between about 17 times (17×) to about 19 times (19×) its CMC, or between about 17 times (17×) to about 18 times (18×) its CMC.

In some aspects, the DDM is present at a concentration which is between about 18 times (18×) to about 20 times (20×) its CMC, or between about 18 times (18×) to about 19 times (19×) its CMC.

In some aspects, the DDM is present at a concentration which is between about 19 times (19×) to about 20 times (20×) its CMC.

In some aspects, the DDM is present at a concentration between about 5 times (i.e., 5×) to about 10 times (i.e., 10×) its CMC. In some aspects, the DDM is present at a concentration between about 0.0305% (w/v) and about 0.061% (w/v).

In some aspects, the DDM is present at a concentration between about 0.005% (w/v) and about 0.15% (w/v). In some aspects, the DDM is present at a concentration between about 0.03% (w/v) and about 0.06% (w/v). In some aspects, the DDM is present of a concentration of about 0.005% (w/v), about 0.006% (w/v), about 0.007% (w/v), about 0.008% (w/v), about 0.009% (w/v), about 0.01% (w/v), about 0.011% (w/v), about 0.012% (w/v), about 0.013% (w/v), about 0.014% (w/v), about 0.015% (w/v), about 0.016% (w/v), about 0.017% (w/v), about 0.018% (w/v), about 0.019% (w/v), about 0.02% (w/v), about 0.021% (w/v), about 0.022% (w/v), about 0.023% (w/v), about 0.024% (w/v), about 0.025% (w/v), about 0.026% (w/v), about 0.027% (w/v), about 0.028% (w/v), about 0.029% (w/v), about 0.030% (w/v), about 0.031% (w/v), about 0.032% (w/v), about 0.033% (w/v), about 0.034% (w/v), about 0.035% (w/v), about 0.036% (w/v), about 0.037% (w/v), about 0.038% (w/v), about 0.039% (w/v), about 0.04% (w/v), about 0.041% (w/v), about 0.042% (w/v), about 0.043% (w/v), about 0.044% (w/v), about 0.045% (w/v), about 0.046% (w/v), about 0.047% (w/v), about 0.048% (w/v), about 0.049% (w/v), about 0.05% (w/v), about 0.051% (w/v), about 0.052% (w/v), about 0.053% (w/v), about 0.054% (w/v), about 0.055% (w/v), about 0.056% (w/v), about 0.057% (w/v), about 0.058% (w/v), about 0.059% (w/v), about 0.06% (w/v), about 0.061% (w/v), about 0.065% (w/v), about 0.07% (w/v), about 0.075% (w/v), about 0.08% (w/v), about 0.085% (w/v), about 0.09% (w/v), about 0.095% (w/v), about 0.10% (w/v), about 0.110% (w/v), about 0.12% (w/v), about 0.130% (w/v), about 0.14% (w/v), or about 0.150% (w/v).

The CMC of OG is 0.68% w/v. Thus, a 0.1×CMC concentration of OG is 0.068% w/v, a 0.2×CMC concentration of OG is 0.136% (w/v), a 0.3×CMC concentration of OG is 0.204% (w/v), a 0.4×CMC concentration of OG is 0272% (w/v), a 0.5×CMC concentration of OG is 0.34% (w/v), a 0.6×CMC concentration of OG is 0.408% (w/v), a 0.7×CMC concentration of OG is 0476% (w/v), a 0.8×CMC concentration of OG is 0.544% (w/v), a 0.9×CMC concentration of OG is 0.612% (w/v), a 1×CMC concentration of OG is 0.68% (w/v), a 1.1×CMC concentration of OG is 0.748% (w/v), a 1.2×CMC concentration of OG is 0.816% (w/v), a 1.3×CMC concentration of OG is 0.884% (w/v), a 1.4×CMC concentration of OG is 0.952% (w/v), a 1.5×CMC concentration of OG is 1.02% (w/v), a 1.6×CMC concentration of OG is 1.088% (w/v), a 1.7×CMC concentration of OG is 1.156% (w/v), a 1.8×CMC concentration of OG is 1.224% (w/v), a 1.9×CMC concentration of OG is 1.292% (w/v), a 2×CMC concentration of OG is 1.36% (w/v), a 3×CMC concentration of OG is 2.04% (w/v), a 4×CMC concentration of OG is 2.72% (w/v), a 5×CMC concentration of OG is 3.4% (w/v), a 6×CMC concentration of OG is 4.08% (w/v), a 7×CMC concentration of OG is 4.76% (w/v), a 8×CMC concentration of OG is 5.44% (w/v), a 9×CMC concentration of OG is 6.12% (w/v), and a 10×CMC concentration of OG is 6.8% (w/v). The molecular weight of OG is 292.37 g/mol. Thus, a person of ordinary skill in the art could easily convert the disclosed % (w/v) concentrations to molar concentrations.

In some aspects, the OG is present at a concentration which is at least about 0.1 times (0.1×) its CMC, at least about 0.2 times (0.2×) its CMC, at least about 0.3 times (0.3×) its CMC, at least about 0.4 times (0.4×) its CMC, at least about 0.5 times (0.5×) its CMC, at least about 0.6 (0.6×) times its CMC, at least about 0.7 times (0.7×) its CMC, at least about 0.8 times (0.8×) its CMC, at least about 0.9 times (0.9×) its CMC, at least about 1 time (1×) its CMC, at least about 1.1 times (1.1×) its CMC, at least about 1.2 times (1.2×) its CMC, at least about 1.3 times (1.3×) its CMC, at least about 1.4 times (1.4×) its CMC, at least about 1.5 times (1.5×) its CMC, at least about 1.6 times (1.6×) its CMC, at least about 1.7 times (1.7×) its CMC, at least about 1.8 times (1.8×) its CMC, at least about 1.9 times (1.9×) its CMC, at least about 2 times (2×) its CMC, at least about 3 times (3×) its CMC, at least about 4 times (4×) its CMC, at least about 5 times (5×) its CMC, at least about 6 times (6×) its CMC, at least about 7 times (7×) its CMC, at least about 8 times (8×) its CMC, at least about 9 times (9×) its CMC, or at least about 10 times (10×) its CMC.

In some aspects, the detergent combination of the present disclosure comprises OG present at a concentration which is at least about 0.068% (w/v), at least about 0.136% (w/v), at least about 0.204%, (w/v) at least about 0.272% (w/v), at least about 0.340% (w/v), at least about 0.408% (w/v), at least about 0.476% (w/v), at least about 0.544% (w/v), at least about 0.612% (w/v), at least about 0.680% (w/v), at least about 0.748% (w/v), at least about 0.816% (w/v), at least about 0.884% (w/v), at least about 0.952% (w/v), at least about 1.020% (w/v), at least about 1.088% (w/v), at least about 1.156% (w/v), at least about 1.224% (w/v), at least about 1.292% (w/v), at least about 1.360% (w/v), at least about 2.04% (w/v), at least about 2.72% (w/v), at least about 3.4% (w/v), at least about 4.08% (w/v), at least about 4.76% (w/v), at least about 5.44% (w/v), at least about 6.12% (w/v), or at least about 6.8% (w/v).

In some aspects, the OG is present at a concentration which is between about 0.1 times (0.1×) to about 10 times (10×) its CMC, between about 0.1 times (0.1×) to about 9 times (9×) its CMC, between about 0.1 times (0.1×) to about 8 times (8×) its CMC, between about 0.1 times (0.1×) to about 7 times (7×) its CMC, between about 0.1 times (0.1×) to about 6 times (6×) its CMC, between about 0.1 times (0.1×) to about 5 times (5×) its CMC, between about 0.1 times (0.1×) to about 4 times (4×) its CMC, between about 0.1 times (0.1×) to about 3 times (3×) its CMC, between 0.1 times (0.1×) to about 2 times (2×) its CMC, between about 0.1 times (0.1×) to about 1.9 times (1.9×) its CMC, between about 0.1 times (0.1×) to about 1.8 times (1.8×) its CMC, between about 0.1 times (0.1×) to about 1.7 times (1.7×) its CMC, between about 0.1 times (0.1×) to about 1.6 times (1.6×) its CMC, between about 0.1 times (0.1×) to about 1.5 times (1.5×) its CMC, between about 0.1 times (0.1×) to about 1.4 times (1.4×) its CMC, between about 0.1 times (0.1×) to about 1.3 times (1.3×) its CMC, between about 0.1 times (0.1×) to about 1.2 times (1.2×) its CMC, between about 0.1 times (0.1×) to about 1.1 times (1.1×) its CMC, between about 0.1 times (0.1×) to about 1.0 times (1.0×) its CMC, between about 0.1 times (0.1×) to about 0.9 times (0.9×) its CMC, between about 0.1 times (0.1×) to about 0.8 times (0.8×) its CMC, between about 0.1 times (0.1×) to about 0.7 times (0.7×) its CMC, between about 0.1 times (0.1×) to about 0.6 times (0.6×) its CMC, between about 0.1 times (0.1×) to about 0.5 times (0.5×) its CMC, between about 0.1 times (0.1×) to about 0.4 times (0.4×) its CMC, between about 0.1 times (0.1×) to about 0.3 times (0.3×) its CMC, or between about 0.1 times (0.1×) to about 0.2 times (0.2×) its CMC.

In some aspects, the OG is present at a concentration which is between about 0.2 times (0.2×) to about 10 times (10×) its CMC, between about 0.2 times (0.2×) to about 9 times (9×) its CMC, between about 0.2 times (0.2×) to about 8 times (8×) its CMC, between about 0.2 times (0.2×) to about 7 times (7×) its CMC, between about 0.2 times (0.2×) to about 6 times (6×) its CMC, between about 0.2 times (0.2×) to about 5 times (5×) its CMC, between about 0.2 times (0.2×) to about 4 times (4×) its CMC, between about 0.2 times (0.2×) to about 3 times (3×) its CMC, between 0.2 times (0.2×) to about 2 times (2×) its CMC, between about 0.2 times (0.2×) to about 1.9 (1.9×) times its CMC, between about 0.2 times (0.2×) to about 1.8 times (1.8×) its CMC, between about 0.2 times (0.2×) to about 1.7 times (1.7×) its CMC, between about 0.2 times (0.2×) to about 1.6 times (1.6×) its CMC, between about 0.2 times (0.2×) to about 1.5 times (1.5×) its CMC, between about 0.2 times (0.2×) to about 1.4 times (1.4×) its CMC, between about 0.2 times (0.2×) to about 1.3 times (1.3×) its CMC, between about 0.2 times (0.2×) to about 1.2 times (1.2×) its CMC, between about 0.2 times (0.2×) to about 1.1 times (1.1×) its CMC, between about 0.2 times (0.2×) to about 1.0 times (1.0×) its CMC, between about 0.2 times (0.2×) to about 0.9 times (0.9×) its CMC, between about 0.2 times (0.2×) to about 0.8 times (0.8×) its CMC, between about 0.2 times (0.2×) to about 0.7 times (0.7×) its CMC, between about 0.2 times (0.2×) to about 0.6 times (0.6×) its CMC, between about 0.2 times (0.2×) to about 0.5 times (0.5×) its CMC, between about 0.2 times (0.2×) to about 0.4 times (0.4×) its CMC, or between about 0.2 times (0.2×) to about 0.3 times (0.3×) its CMC.

In some aspects, the OG is present at a concentration which is between about 0.3 times (0.3×) to about 10 times (10×) its CMC, between about 0.3 times (0.3×) to about 9 times (9×) its CMC, between about 0.3 times (0.3×) to about 8 times (8×) its CMC, between about 0.3 times (0.3×) to about 7 times (7×) its CMC, between about 0.3 times (0.3×) to about 6 times (6×) its CMC, between about 0.3 times (0.3×) to about 5 times (5×) its CMC, between about 0.3 times (0.3×) to about 4 times (4×) its CMC, between about 0.3 times (0.3×) to about 3 times (3×) its CMC, between times 0.3 (0.3×) to about 2 times (2×) its CMC, between about 0.3 times (0.3×) to about 1.9 times (1.9×) its CMC, between about 0.3 times (0.3×) to about 1.8 times (1.8×) its CMC, between about 0.3 times (0.3×) to about 1.7 times (1.7×) its CMC, between about 0.3 times (0.3×) to about 1.6 times (1.6×) its CMC, between about 0.3 times (0.3×) to about 1.5 times (1.5×) its CMC, between about 0.3 times (0.3×) to about 1.4 times (1.4×) its CMC, between about 0.3 times (0.3×) to about 1.3 times (1.3×) its CMC, between about 0.3 times (0.3×) to about 1.2 times (1.2×) its CMC, between about 0.3 times (0.3×) to about 1.1 times (1.1×) its CMC, between about 0.3 times (0.3×) to about 1.0 times (1.0×) its CMC, between about 0.3 times (0.3×) to about 0.9 times (0.9×) its CMC, between about 0.3 times (0.3×) to about 0.8 times (0.8×) its CMC, between about 0.3 times (0.3×) to about 0.7 times (0.7×) its CMC, between about 0.3 times (0.3×) to about 0.6 times (0.6×) its CMC, between about 0.3 times (0.3×) to about 0.5 times (0.5×) its CMC, or between about 0.3 times (0.3×) to about 0.4 times (0.4×) its CMC.

In some aspects, the OG is present at a concentration which is between about 0.4 times (0.4×) to about 10 times (10×) its CMC, between about 0.4 times (0.4×) to about 9 times (9×) its CMC, between about 0.4 times (0.4×) to about 8 times (8×) its CMC, between about 0.4 times (0.4×) to about 7 times (7×) its CMC, between about 0.4 times (0.4×) to about 6 times (6×) its CMC, between about 0.4 times (0.4×) to about 5 times (5×) its CMC, between about 0.4 times (0.4×) to about 4 times (4×) its CMC, between about 0.4 times (0.4×) to about 3 times (3×) its CMC, between 0.4 times (0.4×) to about 2 times (2×) its CMC, between about 0.4 times (0.4×) to about 1.9 times (1.9×) its CMC, between about 0.4 times (0.4×) to about 1.8 times (1.8×) its CMC, between about 0.4 times (0.4×) to about 1.7 times (1.7×) its CMC, between about 0.4 times (0.4×) to about 1.6 times (1.6×) its CMC, between about 0.4 times (0.4×) to about 1.5 times (1.5×) its CMC, between about 0.4 times (0.4×) to about 1.4 times (1.4×) its CMC, between about 0.4 times (0.4×) to about 1.3 times (1.3×) its CMC, between about 0.4 times (0.4×) to about 1.2 times (1.2×) its CMC, between about 0.4 times (0.4×) to about 1.1 times (1.1×) its CMC, between about 0.4 times (0.4×) to about 1.0 times (1.0×) its CMC, between about 0.4 times (0.4×) to about 0.9 times (0.9×) its CMC, between about 0.4 times (0.4×) to about 0.8 times (0.8×) its CMC, between about 0.4 times (0.4×) to about 0.7 times (0.7×) its CMC, between about 0.4 times (0.4×) to about 0.6 times (0.6×) its CMC, or between about 0.4 times (0.4×) to about 0.5 times (0.5×) its CMC.

In some aspects, the OG is present at a concentration which is between about 0.5 times (0.5×) to about 10 times (10×) its CMC, between about 0.5 times (0.5×) to about 9 times (9×) its CMC, between about 0.5 times (0.5×) to about 8 times (8×) its CMC, between about 0.5 times (0.5×) to about 7 times (7×) its CMC, between about 0.5 times (0.5×) to about 6 times (6×) its CMC, between about 0.5 times (0.5×) to about 5 times (5×) its CMC, between about 0.5 times (0.5×) to about 4 times (4×) its CMC, between about 0.5 times (0.5×) to about 3 times (3×) its CMC, between 0.5 times (0.5×) to about 2 times (2×) its CMC, between about 0.5 times (0.5×) to about 1.9 times (1.9×) its CMC, between about 0.5 times (0.5×) to about 1.8 times (1.8×) its CMC, between about 0.5 times (0.5×) to about 1.7 times (1.7×) its CMC, between about 0.5 times (0.5×) to about 1.6 times (1.6×) its CMC, between about 0.5 times (0.5×) to about 1.5 times (1.5×) its CMC, between about 0.5 times (0.5×) to about 1.4 times (1.4×) its CMC, between about 0.5 times (0.5×) to about 1.3 times (1.3×) its CMC, between about 0.5 times (0.5×) to about 1.2 times (1.2×) its CMC, between about 0.5 times (0.5×) to about 1.1 times (1.1×) its CMC, between about 0.5 times (0.5×) to about 1.0 times (1.0×) its CMC, between about 0.5 times (0.5×) to about 0.9 times (0.9×) its CMC, between about 0.5 times (0.5×) to about 0.8 times (0.8×) its CMC, between about 0.5 times (0.5×) to about 0.7 times (0.7×) its CMC, or between about 0.5 times (0.5×) to about 0.6 times (0.6×) its CMC.

In some aspects, the OG is present at a concentration which is between about 0.6 times (0.6×) to about 10 times (10×) its CMC, between about 0.6 times (0.6×) to about 9 times (9×) its CMC, between about 0.6 times (0.6×) to about 8 times (8×) its CMC, between about 0.6 times (0.6×) to about 7 times (7×) its CMC, between about 0.6 times (0.6×) to about 6 times (6×) its CMC, between about 0.6 times (0.6×) to about 5 times (5×) its CMC, between about 0.6 times (0.6×) to about 4 times (4×) its CMC, between about 0.6 times (0.6×) to about 3 times (3×) its CMC, between about 0.6 times (0.6×) to about 2 times (2×) its CMC, between about 0.6 times (0.6×) to about 1.9 times (1.9×) its CMC, between about 0.6 times (0.6×) to about 1.8 times (1.8×) its CMC, between about 0.6 times (0.6×) to about 1.7 times (1.7×) its CMC, between about 0.6 times (0.6×) to about 1.6 times (1.6×) its CMC, between about 0.6 times (0.6×) to about 1.5 times (1.5×) its CMC, between about 0.6 times (0.6×) to about 1.4 times (1.4×) its CMC, between about 0.6 times (0.6×) to about 1.3 times (1.3×) its CMC, between about 0.6 times (0.6×) to about 1.2 times (1.2×) its CMC, between about 0.6 times (0.6×) to about 1.1 times (1.1×) its CMC, between about 0.6 times (0.6×) to about 1.0 times (1.0×) its CMC, between about 0.6 times (0.6×) to about 0.9 times (0.9×) its CMC, between about 0.6 times (0.6×) to about 0.8 times (0.8×) its CMC, or between about 0.6 times (0.6×) to about 0.7 times (0.7×) its CMC.

In some aspects, the OG is present at a concentration which is between about 0.7 times (0.7×) to about 10 times (10×) its CMC, between about 0.7 times (0.7×) to about 9 times (9×) its CMC, between about 0.7 times (0.7×) to about 8 times (8×) its CMC, between about 0.7 times (0.7×) to about 7 times (7×) its CMC, between about 0.7 times (0.7×) to about 6 times (6×) its CMC, between about 0.7 times (0.7×) to about 5 times (5×) its CMC, between about 0.7 times (0.7×) to about 4 times (4×) its CMC, between about 0.7 times (0.7×) to about 3 times (3×) its CMC, between about 0.7 times (0.7×) to about 2 times (2×) its CMC, between about 0.7 times (0.7×) to about 1.9 times (1.9×) its CMC, between about 0.7 times (0.7×) to about 1.8 times (1.8×) its CMC, between about 0.7 times (0.7×) to about 1.7 times (1.7×) its CMC, between about 0.7 times (0.7×) to about 1.6 times (1.6×) its CMC, between about 0.7 times (0.7×) to about 1.5 times (1.5×) its CMC, between about 0.7 times (0.7×) to about 1.4 times (1.4×) its CMC, between about 0.7 times (0.7×) to about 1.3 times (1.3×) its CMC, between about 0.7 times (0.7×) to about 1.2 times (1.2×) its CMC, between about 0.7 times (0.7×) to about 1.1 times (1.1×) its CMC, between about 0.7 times (0.7×) to about 1.0 times (1.0×) its CMC, between about 0.7 times (0.7×) to about 0.9 times (0.9×) its CMC, or between about 0.7 times (0.7×) to about 0.8 times (0.8×) its CMC.

In some aspects, the OG is present at a concentration which is between about 0.8 times (0.8×) to about 10 times (10×) its CMC, between about 0.8 times (0.8×) to about 9 times (9×) its CMC, between about 0.8 times (0.8×) to about 8 times (8×) its CMC, between about 0.8 times (0.8×) to about 7 times (7×) its CMC, between about 0.8 times (0.8×) to about 6 times (6×) its CMC, between about 0.8 times (0.8×) to about 5 times (5×) its CMC, between about 0.8 times (0.8×) to about 4 times (4×) its CMC, between about 0.8 times (0.8×) to about 3 times (3×) its CMC, between about 0.8 times (0.8×) to about 2 times (2×) its CMC, between about 0.8 times (0.8×) to about 1.9 times (1.9×) its CMC, between about 0.8 times (0.8×) to about 1.8 times (1.8×) its CMC, between about 0.8 times (0.8×) to about 1.7 times (1.7×) its CMC, between about 0.8 times (0.8×) to about 1.6 times (1.6×) its CMC, between about 0.8 times (0.8×) to about 1.5 times (1.5×) its CMC, between about 0.8 times (0.8×) to about 1.4 times (1.4×) its CMC, between about 0.8 times (0.8×) to about 1.3 times (1.3×) its CMC, between about 0.8 times (0.8×) to about 1.2 times (1.2×) its CMC, between about 0.8 times (0.8×) to about 1.1 times (1.1×) its CMC, between about 0.8 times (0.8×) to about 1.0 times (1.0×) its CMC, or between about 0.8 times (0.8×) to about 0.9 times (0.9×) its CMC.

In some aspects, the OG is present at a concentration which is between about 0.9 times (0.9×) to about 10 times (10×) its CMC, between about 0.9 times (0.9×) to about 9 times (9×) its CMC, between about 0.9 times (0.9×) to about 8 times (8×) its CMC, between about 0.9 times (0.9×) to about 7 times (7×) its CMC, between about 0.9 times (0.9×) to about 6 times (6×) its CMC, between about 0.9 times (0.9×) to about 5 times (5×) its CMC, between about 0.9 times (0.9×) to about 4 times (4×) its CMC, between about 0.9 times (0.9×) to about 3 times (3×) its CMC, between 0.9 times (0.9×) to about 2 times (2×) its CMC, between about 0.9 times (0.9×) to about 1.9 times (1.9×) its CMC, between about 0.9 times (0.9×) to about 1.8 times (1.8×) its CMC, between about 0.9 times (0.9×) to about 1.7 times (1.7×) its CMC, between about 0.9 times (0.9×) to about 1.6 times (1.6×) its CMC, between about 0.9 times (0.9×) to about 1.5 times (1.5×) its CMC, between about 0.9 times (0.9×) to about 1.4 times (1.4×) its CMC, between about 0.9 times (0.9×) to about 1.3 times (1.3×) its CMC, between about 0.9 times (0.9×) to about 1.2 times (1.2×) its CMC, between about 0.9 times (0.9×) to about 1.1 times (1.1×) its CMC, or between about 0.9 times (0.9×) to about 1.0 times (1.0×) its CMC.

In some aspects, the OG is present at a concentration which is between about 1 times (1×) to about 10 times (10×) its CMC, between about 1 times (1×) to about 9 times (9×) its CMC, between about 1 times (1×) to about 8 times (8×) its CMC, between about 1 times (1×) to about 7 times (7×) its CMC, between about 1 times (1×) to about 6 times (6×) its CMC, between about 1 times (1×) to about 5 times (5×) its CMC, between about 1 times (1×) to about 4 times (4×) its CMC, between about 1 times (1×) to about 3 times (3×) its CMC, between 1 times (1×) to about 2 times (2×) its CMC, between about 1 times (1×) to about 1.9 times (1.9×) its CMC, between about 1 times (1×) to about 1.8 times (1.8×) its CMC, between about 1 times (1×) to about 1.7 times (1.7×) its CMC, between about 1 times (1×) to about 1.6 times (1.6×) its CMC, between about 1 times (1×) to about 1.5 times (1.5×) its CMC, between about 1 times (1×) to about 1.4 times (1.4×) its CMC, between about 1 times (1×) to about 1.3 times (1.3×) its CMC, between about 1 times (1×) to about 1.2 times (1.2×) its CMC, or between about 1 times (1×) to about 1.1 times (1.1×) its CMC.

In some aspects, the OG is present at a concentration which is between about 1.1 times (1.1×) to about 10 times (10×) its CMC, between about 1.1 times (1.1×) to about 9 times (9×) its CMC, between about 1.1 times (1.1×) to about 8 times (8×) its CMC, between about 1.1 times (1.1×) to about 7 times (7×) its CMC, between about 1.1 times (1.1×) to about 6 times (6×) its CMC, between about 1.1 times (1.1×) to about 5 times (5×) its CMC, between about 1.1 times (1.1×) to about 4 times (4×) its CMC, between about 1.1 times (1.1×) to about 3 times (3×) its CMC, between 1.1 times (1.1×) to about 2 times (2×) its CMC, between about 1.1 times (1.1×) to about 1.9 times (1.9×) its CMC, between about 1.1 times (1.1×) to about 1.8 times (1.8×) its CMC, between about 1.1 times (1.1×) to about 1.7 times (1.7×) its CMC, between about 1.1 times (1.1×) to about 1.6 times (1.6×) its CMC, between about 1.1 times (1.1×) to about 1.5 times (1.5×) its CMC, between about 1.1 times (1.1×) to about 1.4 times (1.4×) its CMC, between about 1.1 times (1.1×) to about 1.3 times (1.3×) its CMC, or between about 1.1 times (1.1×) to about 1.2 (1.2×) its CMC.

In some aspects, the OG is present at a concentration which is between about 1.2 times (1.2×) to about 10 times (10×) its CMC, between about 1.2 times (1.2×) to about 9 times (9×) its CMC, between about 1.2 times (1.2×) to about 8 times (8×) its CMC, between about 1.2 times (1.2×) to about 7 times (7×) its CMC, between about 1.2 times (1.2×) to about 6 times (6×) its CMC, between about 1.2 times (1.2×) to about 5 times (5×) its CMC, between about 1.2 times (1.2×) to about 4 times (4×) its CMC, between about 1.2 times (1.2×) to about 3 times (3×) its CMC, between 1.2 times (1.2×) to about 2 times (2×) its CMC, between about 1.2 times (1.2×) to about 1.9 times (1.9×) its CMC, between about 1.2 times (1.2×) to about 1.8 times (1.8×) its CMC, between about 1.2 times (1.2×) to about 1.7 times (1.7×) its CMC, between about 1.2 times (1.2×) to about 1.6 times (1.6×) its CMC, between about 1.2 times (1.2×) to about 1.5 times (1.5×) its CMC, between about 1.2 times (1.2×) to about 1.4 times (1.4×) its CMC, or between about 1.2 times (1.2×) to about 1.3 times (1.3×) its CMC.

In some aspects, the OG is present at a concentration which is between about 1.3 times (1.3×) to about 10 times (10×) its CMC, between about 1.3 times (1.3×) to about 9 times (9×) its CMC, between about 1.3 times (1.3×) to about 8 times (8×) its CMC, between about 1.3 times (1.3×) to about 7 times (7×) its CMC, between about 1.3 times (1.3×) to about 6 times (6×) its CMC, between about 1.3 times (1.3×) to about 5 times (5×) its CMC, between about 1.3 times (1.3×) to about 4 times (4×) its CMC, between about 1.3 times (1.3×) to about 3 times (3×) its CMC, between 1.3 times (1.3×) to about 2 times (2×) its CMC, between about 1.3 times (1.3×) to about 1.9 times (1.9×) its CMC, between about 1.3 times (1.3×) to about 1.8 times (1.8×) its CMC, between about 1.3 times (1.3×) to about 1.7 times (1.7×) its CMC, between about 1.3 times (1.3×) to about 1.6 times (1.6×) its CMC, between about 1.3 times (1.3×) to about 1.5 times (1.5×) its CMC, or between about 1.3 times (1.2×) to about 1.4 times (1.4×) its CMC.

In some aspects, the OG is present at a concentration which is between about 1.4 times (1.4×) to about 10 times (10×) its CMC, between about 1.4 times (1.4×) to about 9 times (9×) its CMC, between about 1.4 times (1.4×) to about 8 times (8×) its CMC, between about 1.4 times (1.4×) to about 7 times (7×) its CMC, between about 1.4 times (1.4×) to about 6 times (6×) its CMC, between about 1.4 times (1.4×) to about 5 times (5×) its CMC, between about 1.4 times (1.4×) to about 4 times (4×) its CMC, between about 1.4 times (1.4×) to about 3 times (3×) its CMC, between 1.4 times (1.4×) to about 2 times (2×) its CMC, between about 1.4 times (1.4×) to about 1.9 times (1.9×) its CMC, between about 1.4 times (1.4×) to about 1.8 times (1.8×) its CMC, between about 1.4 times (1.4×) to about 1.7 times (1.7×) its CMC, between about 1.4 times (1.4×) to about 1.6 times (1.6×) its CMC, or between about 1.4 times (1.4×) to about 1.5 times (1.5×) its CMC.

In some aspects, the OG is present at a concentration which is between about 1.5 times (1.5×) to about 10 times (10×) its CMC, between about 1.5 times (1.5×) to about 9 times (9×) its CMC, between about 1.5 times (1.5×) to about 8 times (8×) its CMC, between about 1.5 times (1.5×) to about 7 times (7×) its CMC, between about 1.5 times (1.5×) to about 6 times (6×) its CMC, between about 1.5 times (1.5×) to about 5 times (5×) its CMC, between about 1.5 times (1.5×) to about 4 times (4×) its CMC, between about 1.5 times (1.5×) to about 3 times (3×) its CMC, between 1.5 times (1.5×) to about 2 times (2×) its CMC, between about 1.5 times (1.5×) to about 1.9 times (1.9×) its CMC, between about 1.5 times (1.5×) to about 1.8 times (1.8×) its CMC, between about 1.5 times (1.5×) to about 1.7 times (1.7×) its CMC, or between about 1.5 times (1.5×) to about 1.6 times (1.6×) its CMC.

In some aspects, the OG is present at a concentration which is between about 1.6 times (1.6×) to about 10 times (10×) its CMC, between about 1.6 times (1.6×) to about 9 times (9×) its CMC, between about 1.6 times (1.6×) to about 8 times (8×) its CMC, between about 1.6 times (1.6×) to about 7 times (7×) its CMC, between about 1.6 times (1.6×) to about 6 times (6×) its CMC, between about 1.6 times (1.6×) to about 5 times (5×) its CMC, between about 1.6 times (1.6×) to about 4 times (4×) its CMC, between about 1.6 times (1.6×) to about 3 times (3×) its CMC, between 1.6 times (1.6×) to about 2 times (2×) its CMC, between about 1.6 times (1.6×) to about 1.9 times (1.9×) its CMC, between about 1.6 times (1.6×) to about 1.8 times (1.8×) its CMC, or between about 1.6 times (1.6×) to about 1.7 times (1.7×) its CMC.

In some aspects, the OG is present at a concentration which is between about 1.7 times (1.7×) to about 10 times (10×) its CMC, between about 1.7 times (1.7×) to about 9 times (9×) its CMC, between about 1.7 times (1.7×) to about 8 times (8×) its CMC, between about 1.7 times (1.7×) to about 7 times (7×) its CMC, between about 1.7 times (1.7×) to about 6 times (6×) its CMC, between about 1.7 times (1.7×) to about 5 times (5×) its CMC, between about 1.7 times (1.7×) to about 4 times (4×) its CMC, between about 1.7 times (1.7×) to about 3 times (3×) its CMC, between 1.7 times (1.7×) to about 2 times (2×) its CMC, between about 1.7 times (1.7×) to about 1.9 times (1.9×) its CMC, or between about 1.7 times (1.7×) to about 1.8 times (1.8×) its CMC.

In some aspects, the OG is present at a concentration which is between about 1.8 times (1.8×) to about 10 times (10×) its CMC, between about 1.8 times (1.8×) to about 9 times (9×) its CMC, between about 1.8 times (1.8×) to about 8 times (8×) its CMC, between about 1.8 times (1.8×) to about 7 times (7×) its CMC, between about 1.8 times (1.8×) to about 6 times (6×) its CMC, between about 1.8 times (1.8×) to about 5 times (5×) its CMC, between about 1.8 times (1.8×) to about 4 times (4×) its CMC, between about 1.8 times (1.8×) to about 3 times (3×) its CMC, between 1.8 times (1.8×) to about 2 times (2×) its CMC, or between about 1.8 times (1.8×) to about 1.9 (1.9×) its CMC.

In some aspects, the OG is present at a concentration which is between about 1.9 times (1.9×) to about 10 times (10×) its CMC, between about 1.9 times (1.9×) to about 9 times (9×) its CMC, between about 1.9 times (1.9×) to about 8 times (8×) its CMC, between about 1.9 times (1.9×) to about 7 times (7×) its CMC, between about 1.9 times (1.9×) to about 6 times (6×) its CMC, between about 1.9 times (1.9×) to about 5 times (5×) its CMC, between about 1.9 times (1.9×) to about 4 times (4×) its CMC, between about 1.9 times (1.9×) to about 3 times (3×) its CMC, or between 1.9 times (1.9×) to about 2 times (2×) its CMC.

In some aspects, the OG is present at a concentration which is between about 2 times (2×) to about 10 times (10×) its CMC, between about 2 times (2×) to about 9 times (9×) its CMC, between about 2 times (2×) to about 8 times (8×) its CMC, between about 2 times (2×) to about 7 times (7×) its CMC, between about 2 times (2×) to about 6 times (6×) its CMC, between about 2 times (2×) to about 5 times (5×) its CMC, between about 2 times (2×) to about 4 times (4×) its CMC, or between about 2 times (2×) to about 3 times (3×) its CMC.

In some aspects, the OG is present at a concentration which is between about 3 times (3×) to about 10 times (10×) its CMC, between about 3 times (3×) to about 9 times (9×) its CMC, between about 3 times (3×) to about 8 times (8×) its CMC, between about 3 times (3×) to about 7 times (7×) its CMC, between about 3 times (3×) to about 6 times (6×) its CMC, between about 3 times (3×) to about 5 times (5×) its CMC, or between about 3 times (3×) to about 4 times (4×) its CMC.

In some aspects, the OG is present at a concentration which is between about 4 times (4×) to about 10 times (10×) its CMC, between about 4 times (4×) to about 9 times (9×) its CMC, between about 4 times (4×) to about 8 times (8×) its CMC, between about 4 times (4×) to about 7 times (7×) its CMC, between about 4 times (4×) to about 6 times (6×) its CMC, or between about 4 times (4×) to about 5 times (5×) its CMC.

In some aspects, the OG is present at a concentration which is between about 5 times (5×) to about 10 times (10×) its CMC, between about 5 times (5×) to about 9 times (9×) its CMC, between about 5 times (5×) to about 8 times (8×) its CMC, between about 5 times (5×) to about 7 times (7×) its CMC, or between about 5 times (5×) to about 6 times (6×) its

In some aspects, the OG is present at a concentration which is between about 6 times (6×) to about 10 times (10×) its CMC, between about 6 times (6×) to about 9 times (9×) its CMC, between about 6 times (6×) to about 8 times (8×) its CMC, between about 6 times (6×) to about 7 times (7×) its CMC.

In some aspects, the OG is present at a concentration which is between about 7 times (7×) to about 10 times (10×) its CMC, between about 7 times (7×) to about 9 times (9×) its CMC, or between about 7 times (7×) to about 8 times (8×) its CMC.

In some aspects, the OG is present at a concentration which is between about 8 times (8×) to about 10 times (10×) its CMC, or between about 8 times (8×) to about 9 times (9×) its CMC.

In some aspects, the OG is present at a concentration between about 0.1 times (i.e., 0.1×) to about 1 times (i.e., 1×) its CMC. In some aspects, the OG is present at a concentration between about 0.068% (w/v) and about 0.68% (w/v).

In some aspects, the OG is present at a concentration between about 0.1 times (i.e., 0.1×) to about 1 times (i.e., 1×) its CMC. In some aspects, the OG is present at a concentration between about 0.068% (w/v) and about 0.68% (w/v).

In some aspects, the OG is present at a concentration between about 0.05% (w/v) and about 0.75% (w/v). In some aspects, the OG is present at a concentration between about 0.07% (w/v) and about 0.7% (w/v). In some aspects, the OG is present of a concentration of about 0.005% (w/v), about 0.06% (w/v), about 0.07% (w/v), about 0.08% (w/v), about 0.09% (w/v), about 0.1% (w/v), about 0.11% (w/v), about 0.12% (w/v), about 0.130% (w/v), about 0.14% (w/v), about 0.15% (w/v), about 0.16% (w/v), about 0.17% (w/v), about 0.18% (w/v), about 0.19% (w/v), about 0.2% (w/v), about 0.21% (w/v), about 0.22% (w/v), about 0.23% (w/v), about 0.24% (w/v), about 0.25% (w/v), about 0.26% (w/v), about 0.27% (w/v), about 0.28% (w/v), about 0.29% (w/v), about 0.30% (w/v), about 0.31% (w/v), about 0.32% (w/v), about 0.33% (w/v), about 0.34% (w/v), about 0.35% (w/v), about 0.36% (w/v), about 0.37% (w/v), about 0.38% (w/v), about 0.39% (w/v), about 0.4% (w/v), about 0.41% (w/v), about 0.42% (w/v), about 0.43% (w/v), about 0.44% (w/v), about 0.45% (w/v), about 0.46% (w/v), about 0.47% (w/v), about 0.48% (w/v), about 0.49% (w/v), about 0.5% (w/v), about 0.51% (w/v), about 0.52% (w/v), about 0.53% (w/v), about 0.54% (w/v), about 0.55% (w/v), about 0.56% (w/v), about 0.57% (w/v), about 0.58% (w/v), about 0.59% (w/v), about 0.6% (w/v), about 0.65% (w/v), about 0.70% (w/v), about 0.75% (w/v), about 0.80% (w/v), about 0.85% (w/v), about 0.90% (w/v), about 0.95% (w/v), about 1% (w/v), about 1.25% (w/v), about 1.5% (w/v), about 1.75% (w/v), about 2% (w/v), about 2.5% (w/v), about 3% (w/v), about 3% (w/v), about 3.5% (w/v), about 4% (w/v), about 4.5% (w/v), about 5% (w/v), about 5.5% (w/v), about 6% (w/v), about 6.5% (w/v), about 7% (w/v), about 7.5% (w/v), about 8% (w/v), about 8.5% (w/v), about 9% (w/v), about 9.5% (w/v), or about 10% (w/v).

In some aspects, the OG is present at a concentration which is about 0.5 times (0.5×) its CMC, and DDM is present at a concentration which is between about 5 times (5×) to about 10 times (10×) its CMC. In some aspects, the OG is present at a concentration which is about 0.34% (w/v), and DDM is present at a concentration which is between about 0.0305% (w/v) and about 0.061% (w/v).

In some aspects, (i) the OG is present at a concentration which is about 0.5 times (0.5×) its CMC, and the DDM is present at a concentration which is about 5 times (5×) its CMC, (ii) the OG is present at a concentration which is about 0.5 times (0.5×) its CMC, and the DDM is present at a concentration which is about 7.5 times (7.5×) its CMC, (iii) the OG is present at a concentration which is about 0.5 times (0.5×) its CMC, and the DDM is present at a concentration which is about 10 times (10×) its CMC, or (iv) the OG is present at a concentration which is about 0.75 times (0.75×) its CMC, and the DDM is present at a concentration which is about 5 times (5×) its CMC.

In some aspects, (i) the OG is present at a concentration which is about 0.34% (w/v), and the DDM is present at a concentration which is about 0.0305% (w/v), (ii) the OG is present at a concentration which is about 0.34% (w/v), and the DDM is present at a concentration which is about 0.04575% (w/v), (iii) the OG is present at a concentration which is about 0.34% (w/v), and the DDM is present at a concentration which is about 0.061% (w/v), or (iv) the OG is present at a concentration which is about 0.51% (w/v), and the DDM is present at a concentration which is about 0.0305 (w/v) %.

In some aspects, the detergent combination of the present disclosure comprises DDM and OG at specific concentrations as indicated in the table below. Thus, a detergent combination of the present disclosure can be described as DMMx:OGy wherein x can be any integer between 1 and 21, and y can be any integer between 1 and 28. In some aspects, the detergent combination can be DMM5:OG5, which would correspond to a mixture of DMM at 5 times (5×) its CMC, and OG at 0.5 times (0.5×) its CMC, if the respective concentrations are expressed as CMC (fold) concentrations, or 0.0305% (w/v) of DDM and 0.34% (w/v) of OG if the respective concentrations are expressed as dry weight percentage with respect to the volume of solvent, i.e., % (w/v).

| | Concentration | | | Concentration | |
|---|---|---|---|---|---|
| Detergent | CMC (fold) | %(w/v) | Detergent | CMC (fold) | %(w/v) |
| DDM1 | 1 | 0.00610 | OG1 | 0.1 | 0.068 |
| DDM2 | 2 | 0.01220 | OG2 | 0.2 | 0.136 |
| DDM3 | 3 | 0.01830 | OG3 | 0.3 | 0.204 |
| DDM4 | 4 | 0.02440 | OG4 | 0.4 | 0.272 |
| DDM5 | 5 | 0.03050 | OG5 | 0.5 | 0.340 |
| DDM6 | 6 | 0.03660 | OG6 | 0.6 | 0.408 |
| DDM7 | 7 | 0.04270 | OG7 | 0.7 | 0.476 |
| DDM8 | 7.5 | 0.04575 | OG8 | 0.8 | 0.544 |
| DDM9 | 8 | 0.04880 | OG9 | 0.9 | 0.612 |
| DDM10 | 9 | 0.05490 | OG10 | 1 | 0.680 |
| DDM11 | 10 | 0.06100 | OG11 | 1.1 | 0.748 |
| DDM12 | 11 | 0.06710 | OG12 | 1.2 | 0.816 |
| DDM13 | 12 | 0.07320 | OG13 | 1.3 | 0.884 |
| DDM14 | 13 | 0.07930 | OG14 | 1.4 | 0.952 |
| DDM15 | 14 | 0.08540 | OG15 | 1.5 | 1.020 |
| DDM16 | 15 | 0.09150 | OG16 | 1.6 | 1.088 |
| DDM17 | 16 | 0.09760 | OG17 | 1.7 | 1.156 |
| DDM18 | 17 | 0.10370 | OG18 | 1.8 | 1.224 |
| DDM19 | 18 | 0.10980 | OG19 | 1.9 | 1.292 |
| DDM20 | 19 | 0.11590 | OG20 | 2 | 1.360 |
| DDM21 | 20 | 0.12200 | OG21 | 3 | 2.040 |
| | | | OG22 | 4 | 2.720 |
| | | | OG23 | 5 | 3.400 |
| | | | OG24 | 6 | 4.080 |
| | | | OG25 | 7 | 4.760 |
| | | | OG26 | 8 | 5.440 |
| | | | OG27 | 9 | 6.120 |
| | | | OG28 | 10 | 6.800 |

In some aspects, the methods of viral inactivation disclosed herein comprises using a detergent combination disclosed in the table above to inactivate a lipid-enveloped viruses. In some aspects, the methods of viral inactivation disclosed herein comprises using a detergent combination disclosed in the table above to inactivate a herpesviridae virus. In some aspects, the methods of viral inactivation disclosed herein comprises using a detergent combination disclosed in the table above to inactivate a poxviridae virus. In some aspects, the methods of viral inactivation disclosed herein comprises using a detergent combination disclosed in the table above to inactivate a hepadnaviridae virus. In some aspects, the methods of viral inactivation disclosed herein comprises using a detergent combination disclosed in the table above to inactivate a RNA virus. In some aspects, the methods of viral inactivation disclosed herein comprises using a detergent combination disclosed in the table above to inactivate a flaviviridae virus. In some aspects, the methods of viral inactivation disclosed herein comprises using a detergent combination disclosed in the table above to inactivate a togaviridae virus. In some aspects, the methods of viral inactivation disclosed herein comprises using a detergent combination disclosed in the table above to inactivate a coronaviridae virus. In some aspects, the methods of viral inactivation disclosed herein comprises using a detergent combination disclosed in the table above to inactivate a deltavirus. In some aspects, the methods of viral inactivation disclosed herein comprises using a detergent combination disclosed in the table above to inactivate a orthomyxoviridae virus. In some aspects, the methods of viral inactivation disclosed herein comprises using a detergent combination disclosed in the table above to inactivate a paramyxoviridae virus. In some aspects, the methods of viral inactivation disclosed herein comprises using a detergent combination disclosed in the table above to inactivate a rhabdoviridae virus. In some aspects, the methods of viral inactivation disclosed herein comprises using a detergent combination disclosed in the table above to inactivate a bunyaviridae virus. In some aspects, the methods of viral inactivation disclosed herein comprises using a detergent combination disclosed in the table above to inactivate a filoviridae virus. In some aspects, the methods of viral inactivation disclosed herein comprises using a detergent combination disclosed in the table above to inactivate a reverse transcribing virus. In some aspects, the methods of viral inactivation disclosed herein comprises using a detergent combination disclosed in the table above to inactivate a retroviridae virus.

In some aspects, the product feedstream comprises a harvest (e.g., from a bioreactor), a load (e.g., the load of a chromatography or filtration column or other filtration device), an eluate (e.g., a chromatography eluate), a filtrate, or a combination thereof. In general, the methods and compositions disclosed herein can be used to inactivate viruses in any solution containing viruses, suspected of containing viruses, or susceptible of containing viruses. In some aspects, the feedstream is a harvested cell culture fluid. In some aspects, the feedstream comprises a capture pool or a recovered product pool. In some aspects, the capture pool or recovered product pool is a chromatography pool. In some aspects, the capture pool or recovered product pool is an affinity chromatography pool. In some aspects, the capture pool or recovered product pool is a protein A pool, a protein G pool or a protein L pool. In some aspects, the product feedstream is a Protein A chromatography column eluate. In some aspects, the product feedstream is a filtrate, e.g., from a filtering step in a downstream purification process. In some aspects, the product feedstream is a load, e.g., the load of a chromatography column or a filtration system.

In some aspects, the present disclosure provides a method of inactivating virus in a product feedstream in a manufacturing process of a therapeutic protein using an environmentally compatible detergent combination disclosed herein, wherein the feedstream (e.g., a harvest, load, eluate, or filtrate) is subject to chromatography after addition of the detergent combination. In some aspects, the chromatography is one or more of one or more of an affinity chromatography, an ion exchange chromatography (e.g., cation exchange and/or anion exchange), a hydrophobic interaction chromatography, a hydroxyapatite chromatography, or a mixed mode chromatography.

Examples of affinity chromatography materials include, but are not limited to chromatography materials derivatized with protein A or protein G. Examples of affinity chromatography material include, but are not limited to, Prosep-VA, Prosep-VA Ultra Plus, Protein A sepharose fast flow, Tyopearl Protein A. MAbSelect, MAbSelect SuRe and MAbSelect SuRe LX. In some aspects, the affinity chromatography material is an affinity chromatography column. In some aspects, the affinity chromatography material is an affinity chromatography membrane. Examples of anion exchange chromatography materials include, but are not limited to Poros HQ 50, Poros PI 50, Poros D, Mustang Q, Q Sepharose FF, and DEAE Sepharose. Examples of cation exchange materials include, but are not limited to Mustang S, Sartobind S, SO3 Monolith, S Ceramic HyperD, Poros XS, Poros HS50, Poros HS20, SPSFF, SP-Sepharose XL (SPXL), CM Sepharose Fast Flow, Capto S, Fractogel Se HiCap, Fractogel SO3, or Fractogel COO. Examples of HIC chromatography materials include, but are not limited to, Toyopearl hexyl 650, Toyopear butyl 650, Toyopearl phenyl 650, Toyopearl ether 650, Source, Resource, Sepharose Hi-Trap, Octyl sepharose, phenyl sepharose. Examples of hydroxyapatite chromatography material include but are limited to HA Ultrogel, and CHT hydroxyapatite. Examples of mixed mode chromatography materials include, but are not limited to Capto Adhere, QMA, MEP Hypercel, HEA Hypercel, PPA Hypercel, Capto MMC.

Lipid-enveloped viruses which can infect mammalian cells include DNA viruses like a herpesviridae virus, a poxviridae virus, or a hepadnaviridae virus; RNA viruses like a flaviviridae virus, a togaviridae virus, a coronaviridae virus, a deltavirus virus, an orthomyxoviridae virus, a paramyxoviridae virus, a rhabdoviridae virus, a bunyaviridae virus, or a filoviridae virus; and reverse transcribing viruses like a retroviridae virus or a hepadnaviridae virus. Non-limiting examples of lipid-enveloped viruses include a human immunodeficiency virus, a sindbis virus, a herpes simplex virus, a pseudorabies virus, a sendai virus, a vesicular stomatitis virus, a West Nile virus, a bovine viral diarrhea virus, a corona virus, an equine arthritis virus, a severe acute respiratory syndrome virus, Moloney murine leukemia virus, or a vaccinia virus. In some aspects, the lipid-enveloped virus is selected from the group consisting of retroviruses, flaviviruses, orthomyxoviruses, herpes viruses, paramyxoviruses, arena viruses, poxviruses, hepadnaviruses, hepatitis viruses, rhabdoviruses, and togavirus. In some aspect, the virus comprises a lipid-enveloped virus, e.g., a retrovirus such as A-MuLV, a herpesvirus such as HSV-1.

In some aspects, the virus is selected from the group consisting of adenovirus, African swine fever-line virus, arenavirus, arterivirus, astrovirus, baculovirus, badnavirus, barnavirus, birnavirus, bromovirus, bunyavirus, calicivirus, capillovirus, carlavirus, caulimovirus, circovirus, closterovirus, comovirus, coronavirus, cotricovirus, cystovirus, deltavirus, dianthovirus, enamovirus, filovirus, flavivirus, furovirus, fusellovirus, geminivirus, hepadnavirus, herpesvirus, hordeivirus, hypovirus, ideaovirus, inovirus, iridovirus, levivirus, lipothrixvirus, luteovirus, machlomovirus, marafivovirus, microvirus, myovirus, necrovirus, nodavirus, orthomyxovirus, papovavirus, paramyxovirus, partitivirus, parvovirus, phycodnavirus, picornavirus, plamavirus, podovirus, polydnavirus, potexvirus, potyvirus, poxvirus, reovirus, retrovirus, rhabdovirus, rhizidiovirus, sequevirus, siphovirus, sobemovirus, tectivirus, tenuivirus, tetravirus, tobamavirus, tobravirus, togavirus, tombusvirus, totivirus, trichovirus, tymovirus, and umbravirus. In some aspects, the disclosure provides methods for inactivating a subviral agent in a feedstream (e.g., a harvest, load, eluate, or filtrate) comprising subjecting the feedstream to an environmentally compatible detergent combination disclosed herein. In some aspects, the subviral agent is a viroid or a satellite. In some aspect, the present disclosure provides methods for inactivating a virus-like agent in a feedstream (e.g., a harvest, load, eluate, or filtrate) comprising subjecting the feedstream to an environmentally compatible detergent combination disclosed herein.

Virus inactivation can be quantitated using log reduction value (LRV). Thus, in some aspects, the methods of inactivating a virus in a product feedstream (e.g., a harvest, load, eluate, or filtrate) disclosed herein comprise determining a log reduction value (LRV) of the number of virus in the feedstream or virus-containing solution. In some aspects, the LRV value is at least about 4, at least about 5, at least about 6, at least about 7, at least about 8, at least about 9, or at least about 10. In some aspects, LRV is calculated according to the following formula:

$$LRV = - {log}_{10} \frac{{[ Virus in pfu ]}_{P r o d u c t p o o l}}{{[ Virus in pfu ]}_{L o a d / F e e d}} .$$

The term “pfu” or “plaque-forming unit” is a measure used in virology to describe the number of virus particles capable of forming plaques per unit volume. In some aspects, the LRV is at least about 4. In some aspects, the LRV is between about 3 and about 4, between about 4 and about 5, between about 5 and about 6, between about 6 and about 7, between about 7 and about 8, between about 8 and about 9, between about 9 and about 10, between about 3 and about 5, between about 4 and about 6, between about 5 and about 7, between about 6 and about 8, between about 7 and about 9, between about 8 and about 10, between about 3 and about 6, between about 4 and about 7, between about 5 and about 8, between about 6 and about 9, between about 7 and about 10, between about 3 and about 7, between about 4 and about 8, between 5 and about 9, between 6 and about 10, between about 3 and about 8, between 4 and about 9, or between 5 and about 10.

Detecting a viable lipid-coat containing virus can be accomplished by any technique that can qualitatively or quantitatively measure the presence or activity of a viable lipid-coat containing virus. Typically, a cell-culture based assay is used to determine titer levels of a virus, but in vivo infectivity assays can also be employed. Detection of virus amplification may be done, e.g., by microscopic examination (in case of a clearly visible cytopathogenic effect), a PCR-based detection assay, or an antibody-based detection assay. Thus, in some aspects, the LRV is calculated based on an infectivity assay.

One non-limiting example is an in vitro infectivity assay called the Tissue Culture Infectious Dose 50 (TCID50) assay. In this assay, fluid samples and serial dilutions thereof are dispensed into 96-well plates seeded with cells that can serve as hosts for the lipid-coat containing virus being assayed. After inoculation, the plates are incubated at a time and temperature sufficient to allow the virus to replicate in the host cells. After incubation, the cells are examined by microscope for signs of infection, such as, e.g., lysed cells, cells exhibiting a cytopathogenic effect, or any other criteria indicative of viral infection. From the pattern of positive (viral infection) and negative (no viral infection) wells the virus titer is calculated. The absence of any wells showing positive signs of infection is indicative of a fluid that is essentially free of a lipid-coat containing virus. Accordingly, in some aspects, the infectivity assay used to calculate LRV is a TCID50 assay.

Another cell-culture based assay is a plaque assay, where virus-induced effects in the cell culture layer are visible or made visible macroscopically as plaques. The absence of any plaques is indicative of a fluid that is essentially free of a lipid-coat containing virus. In some aspects, the infectivity assay used to calculate LRV is a plaque assay.

In some aspects, the contacting of the detergent combination disclosed herein (e.g., composition comprising DDM and OG) with the product feedstream (e.g., a harvest, load, eluate, or filtrate) or any virus-containing solution or suspected to contain a virus occurs for at least about 10 minutes, at least about 15 minutes, at least about 20 minutes, at least about 25 minutes, at least about 30 minutes, at least about 40 minutes, at least about 50 minutes, at least about 60 minutes, at least about 70 minutes, at least about 80 minutes, at least about 90 minutes, at least about 100 minutes, at least about 110 minutes, or at least about 120 minutes. In some aspects, the contacting of the detergent combination disclosed herein (e.g., composition comprising DDM and OG) with the product feedstream or virus-containing solution or suspected to contain a virus occurs for about 10 minutes, about 15 minutes, about 20 minutes, about 25 minutes, about 30 minutes, about 40 minutes, about 50 minutes, about 60 minutes, about 70 minutes, about 80 minutes, about 90 minutes, about 100 minutes, about 110 minutes, or about 120 minutes. In some aspects, the contacting of the detergent combination disclosed herein (e.g., composition comprising DDM and OG) with the product feedstream or virus-containing solution occurs for between about 10 minutes and about 20 minutes, between about 20 minutes and about 30 minutes, between about 30 minutes and about 40 minutes, between about 40 minutes and about 50 minutes, between about 50 minutes and about 60 minutes, between about 60 minutes and about 70 minutes, between about 70 minutes and about 80 minutes, between about 80 minutes and about 90 minutes, between about 90 minutes and about 100 minutes, between about 100 minutes and about 110 minutes, between about 110 minutes about 120 minutes, between about 15 minutes and about 30 minutes, between about 30 minutes and about 45 minutes, between about 45 minutes and about 60 minutes, between about 60 minutes and about 75 minutes, between about 75 minutes and about 90 minutes, between about 90 minutes and about 105 minutes, between about 105 minutes and about 120 minutes, between about 30 minutes about 60 minutes, between about 60 minutes and about 90 minutes, between about 90 and about 120 minutes, or between about 60 minutes and about 120 minutes. In some aspects, the feedstream can be subjected to a detergent combination disclosed herein for more than 2 hours, e.g., 3 hours, 4 hours, 5 hours, 6 hours, 9 hours, 12 hours, 16 hours, 20 hours, 24 hours, 30 hours, 36 hours, 42 hours, or 48 hours.

In some aspects, the feedstream (e.g., a harvest, load, eluate, or filtrate) is subjected to a detergent combination of the present disclosure at about 4° C. to about 30° C. In some aspects, the feedstream is subjected to the detergent combination of the present disclosure at about 10° C. to about 25° C. In some aspects, the feedstream is subjected to the detergent combination of the present disclosure at about 15° C. to about 20° C. In some aspects, the feedstream is subjected to the detergent combination of the present disclosure at about 20° C. In some aspects, the feedstream is subjected to the detergent combination of the present disclosure at about ambient temperature. In some aspects, the feedstream is subjected to the detergent combination of the present disclosure at about 4° C., 5° C., 10° C., 15° C., 20° C., 25° C., or 30° C.

In some aspects, treatment of the feedstream (e.g., a harvest, load, eluate, or filtrate) in a manufacturing process of a therapeutic protein with an environmentally compatible detergent disclosed herein to inactivate virus in the feedstream does not result in an increase in the amount of protein aggregates (high molecular weight species) beyond the acceptable protein aggregation parameters for total protein in the product feedstream; for example, compared to the manufacturing process without detergent or with Triton X-100.

In some aspects, after the contacting of the detergent combination disclosed herein (e.g., composition comprising DDM and OG) with the product feedstream (e.g., a harvest, load, eluate, or filtrate) or virus-containing solution, the product feedstream or virus-containing solution contains an amount of high molecular weight (HMW) species of the therapeutic protein below about 30%, below about 29%, below about 28%, below about 27%, below about 26%, below about 25%, below about 24%, below about 23%, below about 22%, below about 21%, below about 20%, below about 19%, below about 18%, below about 17%, below about 16%, below about 15%, below about 14%, below about 13%, below about 12%, below about 11%, below about 10%, below about 9%, below about 8%, below about 7%, below about 6%, or below about 5% of the total amount of therapeutic protein.

In some aspects, after the contacting of the detergent combination disclosed herein (e.g., composition comprising DDM and OG) with the product feedstream or virus-containing solution, the product feedstream or virus-containing solution contains an amount of high molecular weight (HMW) species of the therapeutic protein between about 25% and about 30%, e.g., about 25%, about 26%, about 27%, about 28%, about 29%, or about 30%.

In some aspects, treatment of the feedstream (e.g., a harvest, load, eluate, or filtrate) in a manufacturing process of a therapeutic protein with an environmentally compatible detergent disclosed herein to inactivate virus in the feedstream does not result in a change in glycosylation amount beyond the acceptable glycosylation parameters for total protein in the product feedstream; for example, compared to the manufacturing process without detergent or with Triton X-100. In some aspects, treatment of the feedstream in a manufacturing process of a therapeutic protein with an environmentally compatible detergent disclosed herein to inactivate virus in the feedstream does not result in a change in glycosylation pattern; for example, compared to the manufacturing process without detergent or with Triton X-100.

In some aspects, after the contacting of the detergent combination disclosed herein (e.g., composition comprising DDM and OG) with the product feedstream or virus-containing solution, the therapeutic protein has an amount of glycosylation which is the same or has changed (increased or decreased) by about 10%, about 9%, about 8%, about 7%, about 6%, about 5%, about 4%, about 3%, about 2%, or about 1% compared to the amount of glycosylation of the therapeutic protein prior to the contacting.

In some aspects, after the contacting of the detergent combination disclosed herein (e.g., composition comprising DDM and OG) with the product feedstream or virus-containing solution, the therapeutic protein has an amount of N-acetylneuraminic acid (NANA) between about 8 to about 12 moles/mole therapeutic protein, e.g., between about 8 to about 9, between about 9 to about 10, between about 10 to about 11, between about 11 to about 12, between about 8 and about 10, between about 9 and about 11, between 10 and about 12, between about 8 and about 11, or between about 9 and about 12 moles/mole therapeutic protein.

In some aspects, after the contacting of the detergent combination disclosed herein (e.g., composition comprising DDM and OG) with the product feedstream or virus-containing solution, the therapeutic protein has an amount of N-acetylneuraminic acid (NANA) of about 8, about 8.1, about 8.2, about 8.3, about 8.4, about 8.5, about 8.6, about 8.7, about 8.8, about 8.9, or about 9 moles/mole therapeutic protein.

In some aspects, after the contacting of the detergent combination disclosed herein (e.g., composition comprising DDM and OG) with the product feedstream or virus-containing solution, the therapeutic protein has an amount of N-glycolylneuraminic acid (NGNA) less than or equal to about 1.3 moles/mole therapeutic protein.

In some aspects, after the contacting of the detergent combination disclosed herein (e.g., composition comprising DDM and OG) with the product feedstream or virus-containing solution, the therapeutic protein has an amount of N-glycolylneuraminic acid (NGNA) of about 0.6, about 0.7, about 0.8, or about 0.9 moles/mole therapeutic protein.

In some aspects, after the contacting of the detergent combination disclosed herein (e.g., composition comprising DDM and OG) with the product feedstream or virus-containing solution, the therapeutic protein has an amount of N-acetylneuraminic acid (NANA) between about 8 to about 12 moles/mole therapeutic protein (e.g., about 8, about 8.1, about 8.2, about 8.3, about 8.4, about 8.5, about 8.6, about 8.7, about 8.8, about 8.9, or about 9 moles/mole therapeutic protein), and/or an amount of N-glycolylneuraminic acid (NGNA) less than or equal to about 1.3 moles/mole therapeutic protein (e.g., about 0.6, about 0.7, about 0.8, or about 0.9 moles/mole therapeutic protein).

In some aspects, treatment of the feedstream (e.g., a harvest, load, eluate, or filtrate) in a manufacturing process of a therapeutic protein with an environmentally compatible detergent disclosed herein to inactivate virus in the feedstream does not result in an increase in the deamidation of the therapeutic protein product beyond the acceptable protein deamidation parameters for total protein in the product feedstream; for example, compared to the manufacturing process without detergent or with Triton X-100. Deamidation products include proteins where one or more glutamine and/or asparagine residues have been deamidated. In some aspects, deamidation of a product therapeutic protein results in a change in the charge of the polypeptide. Methods to analyze therapeutic proteins for deamidated variants are known in the art. For example, by pH-mediated ion exchange chromatography or isoelectric focusing.

In some aspects, after the contacting of the detergent combination disclosed herein (e.g., composition comprising DDM and OG) with the product feedstream or virus-containing solution, the therapeutic protein has an amount of deamidation of less about 5.9% of the total amount of therapeutic protein, e.g., less than about 5.8%, less than about 5.7%, less than about 5.6%, less that about 5.5%, less than about 5.4%, less than about 5.3%, less than about 5.2%, less than about 5.1%, less than about 5%, less than about 4.9%, less than about 4.8%, less than about 4.7%, less than about 4.7%, less than about 4.6%, less than about 4.5%, less than about 4.4%, less than about 4.3%, less than about 4.2%, less than about 4.1%, less that about 4.1%, less than about 4%, less than about 3.9%, less than about 3.8%, less than about 3.7%, less that about 3.6%, or less than about 3.5% of the total amount of therapeutic protein.

In some aspects, after the contacting of the detergent combination disclosed herein (e.g., composition comprising DDM and OG) with the product feedstream (e.g., a harvest, load, eluate, or filtrate) or virus-containing solution, the therapeutic protein has an amount of deamidation of about 5.9%, about 5.8%, about 5.7%, about 5.6%, about 5.5%, about 5.4%, about 5.3%, about 5.2%, about 5.1%, about 5%, about 4.9%, about 4.8%, about 4.7%, about 4.7%, about 4.6%, about 4.5%, about 4.4%, about 4.3%, about 4.2%, about 4.1%, about 4.1%, about 4%, about 3.9%, about 3.8%, about 3.7%, about 3.6%, or about 3.5% of the total amount of therapeutic protein.

In some aspects, treatment of the feedstream (e.g., a harvest, load, eluate, or filtrate) in a manufacturing process of a therapeutic protein with an environmentally compatible detergent disclosed herein to inactivate virus in the feedstream does not result in an increase in the oxidation of the therapeutic protein product beyond the acceptable protein oxidation parameters for total protein in the product feedstream; for example, compared to the manufacturing process without detergent or with Triton X-100. Oxidation products include proteins where one or more oxygen-reactive amino acid residues, such as methionine, cysteine and tyrosine, have been oxidized. In some aspects, oxidation of a product polypeptide results in a change in the charge of the polypeptide. Methods to analyze polypeptides for oxidized variants are known in the art. For example, levels of oxidation in a given polypeptide may be determined by LC-mass spectroscopy.

In some aspects, after the contacting of the detergent combination disclosed herein (e.g., composition comprising DDM and OG) with the product feedstream (e.g., a harvest, load, eluate, or filtrate) or virus-containing solution, the therapeutic protein has an amount of oxidation of less about 1.3% of the total amount of therapeutic protein, e.g., less than about 1.2%, less than about 1.1%, less than about 1%, less than about 0.9%, less than about 0.8%, less than about 0.7%, or less than about 0.6% of the total amount of therapeutic protein.

In some aspects, after the contacting of the detergent combination disclosed herein (e.g., composition comprising DDM and OG) with the product feedstream or virus-containing solution, the therapeutic protein has an amount of oxidation of about 1%, about 0.9%, about 0.8%, about 0.7%, about 0.6%, or about 0.5% of the total amount of therapeutic protein

In some aspects, treatment of the feedstream (e.g., a harvest, load, eluate, or filtrate) in a manufacturing process of a therapeutic protein with an environmentally compatible detergent disclosed herein to inactivate virus in the feedstream does not result in an increase in process impurities beyond the acceptable protein impurity parameters for total protein in the product feedstream; for example, compared to the manufacturing process without detergent or with Triton X-100. Thus, in some aspects, treatment of the feedstream in a manufacturing process of a therapeutic protein with an environmentally compatible detergent combination disclosed herein to inactivate virus in the feedstream does not alter the clearance of process impurities during the manufacturing process.

For example, treatment of the feedstream (e.g., a harvest, load, eluate, or filtrate) with an environmentally compatible detergent combination disclosed herein does not alter clearance of impurities in the manufacturing process compared to a manufacturing process of the therapeutic protein using Triton X-100 to inactivate virus or a manufacturing process of the therapeutic protein that does not use a detergent. In some aspects, the use of the environmentally compatible detergent combinations disclosed herein does not alter the clearance of process impurities in a particular step in the manufacturing process, such as a chromatography step, a filtration step, a concentration step and the like. In some aspects, the use of the environmentally compatible detergent combinations of the present disclosure does not alter the clearance of process impurities in the overall in the manufacturing process of the therapeutic protein. Process impurities include host cell proteins (HCP), nucleic acids, leached protein A, polypeptides other than the desired polypeptide, endotoxin, viral contaminant, cell culture media component, and variants, fragments, aggregates or derivatives of the desired therapeutic protein.

In some aspects, treatment of the feedstream (e.g., a harvest, load, eluate, or filtrate) in a manufacturing process of a therapeutic protein with an environmentally compatible detergent disclosed herein to inactivate virus in the feedstream does not result in an increase in HCP beyond the acceptable protein HCP levels in the product feedstream; for example, compared to the manufacturing process without detergent or with Triton X-100.

In some aspects, after the contacting of the detergent combination disclosed herein (e.g., composition comprising DDM and OG) with the product feedstream, the product feedstream has a residual amount HCP at a concentration of less than about 5,000 ppm, less than about 4,000 ppm, less than about 3,000 ppm, less than about 2,000 ppm, less than about 1,500 ppm, less than about 1,000 ppm, less than about 900 ppm, less than about 800 ppm, less than about 700 ppm, less than about 600 ppm, or less than about 500 ppm.

In some aspects, after the contacting of the detergent combination disclosed herein (e.g., composition comprising DDM and OG) with the product feedstream, the product feedstream has a residual amount of HCP at a concentration between about 500 ppm and about 2,000 ppm.

In some aspects, after the contacting of the detergent combination disclosed herein (e.g., composition comprising DDM and OG) with the product feedstream, the product feedstream has a residual amount of HCP at a concentration of about 500 ppm, about 600 ppm, about 700 ppm, about 800 ppm, about 900 ppm, about 1,000 ppm, about 1,100 ppm, about 1,200 ppm, about 1,300 ppm, about 1,400 ppm, about 1,500 ppm, about 1,600 ppm, about 1,700 ppm, about 1,800 ppm, about 1,900 ppm or about 2,000 ppm.

In some aspects, treatment of the feedstream in a manufacturing process of a therapeutic protein with an environmentally compatible detergent disclosed herein to inactivate virus in the feedstream does not result in an increase of residual DNA level beyond the acceptable HCP level in the product feedstream; for example, compared to the manufacturing process without detergent or with Triton X-100.

In some aspects, after the contacting of the detergent combination disclosed herein (e.g., composition comprising DDM and OG) with the product feedstream, the product feedstream has a residual amount of DNA at a concentration of less than about 80,000 ppb, less than about 75,000 ppb, less than about 70,000 ppb, less than about 65,000 ppb, less than about 60,000 ppb, less than about 59,000 ppb, less than about 58,000 ppb, less than about 57,000 ppb, or less than about 56,000 ppb.

In some aspects, after the contacting of the detergent combination disclosed herein (e.g., composition comprising DDM and OG) with the product feedstream, the product feedstream has a residual amount of DNA of less than about 500 ppb, less than about 450 ppb, less than about 400 ppb, less than about 350 ppb, less than about 300 ppb, less than about 250 ppb, or less than about 200 ppb.

In some aspects, after the contacting of the detergent combination disclosed herein (e.g., composition comprising DDM and OG) with the product feedstream, the product feedstream has a residual amount of DNA between about 50 and about 200 ppb.

In some aspects, treatment of the feedstream in a manufacturing process of a therapeutic protein with an environmentally compatible detergent disclosed herein to inactivate virus in the feedstream does not result in an increase of residual Protein A level beyond the acceptable Protein A level in the product feedstream; for example, compared to the manufacturing process without detergent or with Triton X-100.

In some aspects, after the contacting of the detergent combination disclosed herein (e.g., composition comprising DDM and OG) with the product feedstream, the product feedstream has a residual amount of Protein A of less than about 1.0 μg/mL, about 0.9 μg/mL, about 0.8 μg/mL, about 0.7 μg/mL, about 0.6 μg/mL, about 0.5 μg/mL, about 0.4 μg/mL, about 0.3 μg/mL, or about 0.2 μg/mL.

In some aspects, the therapeutic protein comprises, e.g., an antibody, an antibody fragment, a fusion protein, a naturally occurring protein, a chimeric protein, or any combination thereof. In some aspects, the therapeutic protein comprises a CTLA4 (cytotoxic T-lymphocyte associated protein 4) domain. In some aspects, the therapeutic protein is a fusion protein, e.g., a fusion protein comprising an Fc portion. In some aspects, the therapeutic protein is a fusion protein comprising an Fc portion and a CTLA4 (cytotoxic T-lymphocyte associated protein 4) domain. In some aspects, the therapeutic protein is abatacept (ORENCIA®) or belatacept (NULOJIX®).

In some aspects, the therapeutic protein is an abatacept composition comprising a polypeptide having an amino acid sequence as set forth in SEQ ID NO:3, a fragment thereof, or a combination thereof. In some aspects, the therapeutic protein is a belatacept composition comprising a polypeptide having an amino acid sequence as set forth in SEQ ID NO:4, a fragment thereof, or a combination thereof.

In some aspects, the predicted environmental concentration (PEC) of detergent in the wastestream following the manufacturing process of the therapeutic protein is the predicted concentration of a detergent in waste material discharged into the receiving water body in environment. In some aspects, the predicted no-effect concentration (PNEC) is the predicted concentration of a detergent in waste material that is safe for discharging to the environment without harmful effects; for example, to the biota of the receiving fresh water and/or marine water. In some aspects, the PEC is less than the PNEC. In some aspects, the PEC is greater than any one of about 0.5-fold, 1-fold, 2-fold, 3-fold, 4-fold, 5-fold, 6-fold, 7-fold, 8-fold, 9-fold, 10-fold, 20-fold, 30-fold, 40 fold, 50-fold, or 100-fold the PNEC.

The present disclosure provides a method of inactivating lipid-enveloped viruses comprising incubating (i) a whole lipid-enveloped virus having envelope proteins with (ii) a detergent combination comprising n-Octyl-β-D-Glucopyranoside (OG) and n-Dodecyl-β-D-Maltopyranoside (DDM) at a OG:DDM concentration selected from the group consisting of 0.5×:5×, 0.5×:7.5×, 0.5×:10×; and 0.75×:5× for a period of time sufficient to inactivate said lipid-enveloped virus.

The present disclosure provides a method in inactivating a lipid-enveloped virus, the method comprising (i) admixing a detergent combination comprising n-Octyl-β-D-Glucopyranoside (OG) and n-Dodecyl-β-D-Maltopyranoside (DDM) at a OG:DDM concentration selected from the group consisting of 0.5×:5×, 0.5×:7.5×, 0.5×:10×; and 0.75×:5× with a fluid comprising a therapeutic protein (e.g., abatacept or belatacept), thereby forming a mixture; and (ii) incubating the mixture for a period of time sufficient to inactivate said lipid-enveloped virus, thereby forming an incubated mixture, wherein the incubated mixture is essentially free of viable lipid-enveloped virus, and where the therapeutic efficacy of the therapeutic protein (e.g., abatacept or belatacept) is preserved.

Also provides is a method of inactivating a lipid-enveloped virus in a product feedstream in a manufacturing process of a therapeutic protein (e.g., abatacept or belatacept), the method comprising the step of subjecting the feedstream to a detergent combination, wherein the detergent combination is environmentally compatible, and wherein the detergent combination comprises n-Octyl-β-D-Glucopyranoside (OG) and n-Dodecyl-β-D-Maltopyranoside (DDM) at a OG:DDM concentration selected from the group consisting of 0.5×:5×, 0.5×:7.5×, 0.5×:10×; and 0.75×:5×. The present disclosure also provides a composition comprising a therapeutic protein product (e.g., abatacept or belatacept) that is essentially free of lipid-enveloped virus prepared according to any of the method of inactivating lipid-enveloped virus disclosed herein.

### II. Therapeutic Proteins

As disclosed above, in some aspects, the therapeutic proteins that can be prepared by using the viral inactivation compositions and methods disclosed herein comprise, for example, antibodies, antibody fragments, Fc portions of antibodies and fusions thereof, antigen binding portions of antibodies, fusion proteins, naturally occurring proteins, recombinant proteins, chimeric proteins, immunoadhesins, enzymes, growth factors, receptors, hormones, regulatory factors, cytokines, or any combination thereof. In some aspects, the therapeutic protein is produced in mammalian cells. In some aspects, the mammalian cell line is a Chinese Hamster Ovary (CHO) cells, or baby hamster kidney (BHK) cells, murine hybridoma cells, or murine myeloma cells. Manufacturing processes of therapeutic proteins using the environmentally compatible (eco-friendly) detergent combination disclosed herein do not adversely affect product quality of the therapeutic protein compared to corresponding processes using Triton X-100.

Any therapeutic protein that is expressible in a host cell may be produced in accordance with the present disclosure and may be present in the compositions provided. The therapeutic protein may be expressed from a gene that is endogenous to the host cell, or from a gene that is introduced into the host cell through genetic engineering. The therapeutic protein may be one that occurs in nature, or may alternatively have a sequence that was engineered or selected by the hand of man. An engineered therapeutic protein may be assembled from other polypeptide segments that individually occur in nature, or may include one or more segments that are not naturally occurring.

The methods and compositions provided may employ any cell that is suitable for growth and/or production of a therapeutic protein in a culture medium, including animal, yeast or insect cells. In one aspect, the cell is any mammalian cell or cell type suitable to cell culture and to expression of polypeptides. The methods provided herein (e.g., methods of inactivating virus) and compositions can therefore employ any suitable type of cell, including an animal cell. In one aspect, the methods and compositions employ a mammalian cell. The methods and compositions may also employ hybridoma cells. In one aspect, the mammalian cell is a non-hybridoma mammalian cell, which has been transformed with exogenous isolated nucleic acid encoding a desired therapeutic protein. In one aspect, the methods and compositions employ mammalian cells selected from the group consisting of human retinoblasts (PER.C6 (CruCell, Leiden, The Netherlands)); monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); baby hamster kidney cells (BHK. ATCC CCL 10); Chinese hamster ovary cells/-DHFR (CHO, Urlaub and Chasin, Proc. Natl. Acad. Sci. USA, 77:4216 (1980)); mouse sertoli cells (TM4, Mather. Biol. Reprod., 23:243-251 (1980)); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 (1982)); MRC 5 cells; FS4 cells; and a human hepatoma line (Hep G2). In some aspects, the methods and compositions employ CHO cells. In some aspects, the culturing of CHO cell lines and expression of therapeutic proteins from CHO cell lines is employed. The therapeutic protein may be secreted into the culture medium from which the therapeutic protein may be isolated and/or purified or the therapeutic protein may be released into the culture medium by lysis of a cell comprising an isolated nucleic acid encoding the therapeutic protein.

In some specific aspects, the therapeutic protein is a CTLA4-Ig molecule, e.g., abatacept or belatacept. The terms “CTLA4-Ig” or “CTLA4-Ig molecule” are used interchangeably, and refer to a protein molecule that comprises at least a polypeptide having a CTLA4 extracellular domain or portion thereof and an immunoglobulin constant region or portion thereof. The extracellular domain and the immunoglobulin constant region can be wild-type, or mutant or modified, and mammalian, including human or mouse. The polypeptide can further comprise additional protein domains. A CTLA4-Ig molecule can also refer to multimer forms of the polypeptide, such as dimers, tetramers, and hexamers. A CTLA4-Ig molecule also is capable of binding to CD80 and/or CD86.

In one aspect, “CTLA4Ig” refers to a protein molecule having the amino acid sequence of residues: (i) 26-383 of SEQ ID NO:1, (ii) 26-382 of SEQ ID NO:1; (iii) 27-383 of SEQ ID NO:1, or (iv) 27-382 of SEQ ID NO:1, or optionally (v) 25-382 of SEQ ID NO:1, or (vi) 25-383 of SEQ ID NO:1. In monomeric form these proteins can be referred to herein as “SEQ ID NO:1 monomers,” or monomers “having a SEQ ID NO:1 sequence”. These SEQ ID NO:1 monomers can dimerize, such that dimer combinations can include, for example: (i) and (i); (i) and (ii); (i) and (iii); (i) and (iv); (i) and (v); (i) and (vi); (ii) and (ii); (ii) and (iii); (ii) and (iv); (ii) and (v); (ii) and (vi); (iii) and (iii); (iii) and (iv); (iii) and (v); (iii) and (vi); (iv) and (iv); (iv) and (v); (iv) and (vi); (v) and (v); (v) and (vi); and, (vi) and (vi). These different dimer combinations can also associate with each other to form tetramer CTLA4Ig molecules. These monomers, dimers, tetramers and other multimers can be referred to herein as “SEQ ID NO:1 proteins” or proteins “having a SEQ ID NO:1 sequence”. (DNA encoding CTLA4Ig as shown in SEQ ID NO:1 was deposited on May 31, 1991 with the American Type Culture Collection (ATCC), 10801 University Blvd., Manassas, Va. 20110-2209 under the provisions of the Budapest Treaty, and has been accorded ATCC accession number ATCC 68629; a Chinese Hamster Ovary (CHO) cell line expressing CTLA4Ig as shown in SEQ ID NO:1 was deposited on May 31, 1991 with ATCC identification number CRL-10762). As utilized herein “Abatacept” refers to SEQ ID NO:1 proteins.

>SEQ ID NO: 1
MGVLLTQRTLLSLVLALLFPSMASMAMHVAQPAVVLASSRGIASFVCEYA
SPGKATEVRVTVLRQADSQVTEVCAATYMMGNELTFLDDSICTGTSSGNQ
VNLTIQGLRAMDTGLYICKVELMYPPPYYLGIGNGTQIYVIDPEPCPDSD
QEPKSSDKTHTSPPSPAPELLGGSSVFLFPPKPKDTLMISRTPEVTCVVV
DVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDK
SRWQQGNVFSCSVMHEALHNHYTQKSLSLSPGK
>SEQ ID NO: 3 (SEQ ID NO: 1 without signal
peptide)
MHVAQPAVVLASSRGIASFVCEYASPGKATEVRVTVLRQADSQVTEVCAA
NTYMMGNELTFLDDSICTGTSSGNQVLTIQGLRAMDTGLYICKVELMYPP
PYYLGIGNGTQIYVIDPEPCPDSDQEPKSSDKTHTSPPSPAPELLGGSSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKS
LSLSPGK

In another aspect, the therapeutic protein is CTLA4-L^{104EA29Y}-Ig (sometimes known as “LEA29Y” or “L104EA29Y”), which is a genetically engineered fusion protein similar in structure to CTAL4-Ig molecule as shown in SEQ ID NO:1. L104EA29Y-Ig has the functional extracellular binding domain of modified human CTLA4 and the Fc domain of human immunoglobulin of the IgG1 class. Two amino acid modifications, leucine to glutamic acid at position 104 (L104E), which is position 130 of SEQ ID NO:1, and alanine to tyrosine at position 29 (A29Y), which is position 55 of SEQ ID NO:1, were made in the B7 binding region of the CTLA4 domain to generate L104EA29Y. SEQ ID NO:2 depict a amino acid sequence of L104EA29YIg comprising a signal peptide; a mutated extracellular domain of CTLA4 starting at methionine at position +27 and ending at aspartic acid at position +150, or starting at alanine at position +26 and ending at aspartic acid at position +150; and an Ig region. DNA encoding L104EA29Y-Ig was deposited on Jun. 20, 2000, with the American Type Culture Collection (ATCC) under the provisions of the Budapest Treaty. It has been accorded ATCC accession number PTA-2104. L104EA29Y-Ig is further described in U.S. Pat. No. 7,094,874, issued on Aug. 22, 2006, and in WO 01/923337 A2, which are incorporated by reference herein in their entireties.

Expression of L104EA29YIg in mammalian cells can result in the production of N- and C-terminal variants, such that the proteins produced can have the amino acid sequence of residues: (i) 26-383 of SEQ ID NO:2, (ii) 26-382 of SEQ ID NO:2; (iii) 27-383 of SEQ ID NO:2 or (iv) 27-382 of SEQ ID NO:2, or optionally (v) 25-382 of SEQ ID NO:2, or (vi) 25-383 of SEQ ID NO:2. In monomeric form these proteins can be referred to herein as “SEQ ID NO:2 monomers,” or monomers “having a SEQ ID NO:2 sequence.”

These proteins can dimerize, such that dimer combinations can include, for example: (i) and (i); (i) and (ii); (i) and (iii); (i) and (iv); (i) and (v); (i) and (vi); (ii) and (ii); (ii) and (iii); (ii) and (iv); (ii) and (v); (ii) and (vi); (iii) and (iii); (iii) and (iv); (iii) and (v); (iii) and (vi); (iv) and (iv); (iv) and (v); (iv) and (vi); (v) and (v); (v) and (vi); and, (vi) and (vi). These different dimer combinations can also associate with each other to form tetramer L104EA29YIg molecules. These monomers, dimers, tetramers and other multimers can be referred to herein as “SEQ ID NO:2 proteins” or proteins “having a SEQ ID NO:2 sequence”. As utilized herein “Belatacept” refers to SEQ ID NO:2 proteins.

>SEQ ID NO: 2
MGVLLTQRTLLSLVLALLFPSMASMAMHVAQPAVVLASSRGIASFVCEYA
SPGKYTEVRVTVLRQADSQVTEVCAATYMMGNELTFLDDSICTGTSSGNQ
VNLTIQGLRAMDTGLYICKVELMYPPPYYEGIGNGTQIYVIDPEPCPDSD
QEPKSSDKTHTSPPSPAPELLGGSSVFLFPPKPKDTLMISRTPEVTCVVV
DVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDK
SRWQQGNVFSCSVMHEALHNHYTQKSLSLSPGK
>SEQ ID NO: 4 (SEQ ID NO: 4 without signal
peptide)
MHVAQPAVVLASSRGIASFVCEYASPGKYTEVRVTVLRQADSQVTEVCAA
TYMMGNELTFLDDSICTGTSSGNQVNLTIQGLRAMDTGLYICKVELMYPP
PYYEGIGNGTQIYVIDPEPCPDSDQEPKSSDKTHTSPPSPAPELLGGSSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKS
LSLSPGK

### III. Methods of Manufacture and Methods of Treatment

The present disclosure also provides a method to treat a disease or condition comprising administering to a subject a therapeutic protein manufactured by a process comprising a viral inactivation step according to the viral inactivation methods disclosed herein, e.g., a viral inactivation method comprising the use of a detergent combination of the present disclosure. Also provided is a pharmaceutical composition manufactured by a process comprising a viral inactivation step according to the viral inactivation methods disclosed herein, e.g., a viral inactivation method comprising the use of a detergent combination of the present disclosure. The present disclosure also provides a method of manufacture a therapeutic protein comprising a viral inactivation step according to the viral inactivation methods disclosed herein, e.g., a viral inactivation method comprising the use of a detergent combination of the present disclosure.

### IV. Kits and Products of Manufacture

The present disclosure also provides a kit or product manufacture comprising a detergent combination disclosed herein, in one or multiple containers (e.g., a separate container for each of the detergents in the detergent combinations disclosed herein) and optionally instructions for inactivating a virus according to the methods disclosed herein. A person of ordinary skill in the art would readily recognize that a detergent combination of the present disclosure or its individual components can be readily incorporated into one of the established kit formats which are well known in the art. In some aspects, the kit or product of manufacture comprises a combination of DDM and OG disclosed herein in solution. In some aspects, the kit or product of manufacture comprises a combination of DDM and OG disclosed herein in dry form. In some aspects, the kit or product of manufacture comprises DDM and OG in separate containers in dry form. In some aspects, the kit or product of manufacture comprises DDM and OG in separate containers in solution. In some aspects, the kit or product of manufacture comprises one or more containers (e.g., vials) comprising DDM, OG, or a combination thereof, in powder form, and one or more containers (e.g., vials) comprising a solvent for reconstitution. In some aspects, the kit or product manufacture comprising instructions for viral inactivation according to the methods of the present disclosure. In some aspects, the kit or product of manufacture comprises instructions to admix DDM and OG to form a detergent combination of the present disclosure.

### EXAMPLES

### Example I

### Assessing Detergent-Mediated Virus Inactivation, Protein Stability and Impurity Clearance

Detergent-mediated virus inactivation (VI) provides a valuable orthogonal strategy for viral clearance particularly for next generation continuous manufacturing. A systematic approach was used to screen detergents as VI agents through the study of VI of three different lipid-enveloped viruses for monoclonal antibodies and fusion proteins. Three major aspects of VI were investigated, namely, the impact of VI agent on the therapeutic quality attributes, clearance of the VI agent and other impurities through subsequent chromatographic steps and lastly the efficacy of VI for the said detergent. Several quality attributes such as charge variance, oxidation, deamidation, glycosylation and aggregation were investigated. Aggregation was a key indicator of stability. Experimental and modeling data was used to decipher the mechanism and kinetics of aggregation for pH sensitive molecules by exploring worst case VI conditions.

Product aggregation and its kinetics were found to be driven by extrinsic factors such as detergent and protein concentration. Aggregation was also impacted by initial aggregation level as well as intrinsic factors such as the protein sequence and detergent hydrophobicity and critical micelle concentration (CMC). VI efficiency was dependent on the virus tested, duration of incubation as well as detergent CMC and concentration. Dodecyl maltopyranoside (DDM) was found to be a suitable candidate for application in VI.

### 1. Introduction

All mammalian manufacturing processes need to effectively remove potential contaminants, such as viruses, other impurities and product degradants to maintain drug efficacy while ensuring patients safety [Bethencourt (2009) Nature Biotechnology 27(8):681-682; Pastoret (2010) Biologicals 38(3):332-334] and compliance with regulatory agencies [Aranha (2012) BioProcess International 10:3; Aranha & Forbes (2001) Pharmaceutical technology 25(4):22-22; Shukla & Aranha (2015) Pharmaceutical Bioprocessing 3(2):127-138]. In addition to testing for presence of viruses in cell lines, viral vectors and reagents, viral clearance (VC) studies are frequently conducted to demonstrate the robustness of the processing stages in removing model and non-specific viruses [Aranha (2012) BioProcess International 10:3; Aranha & Forbes (2001) Pharmaceutical technology 25(4):22-22; Shukla & Aranha (2015) Pharmaceutical Bioprocessing 3(2):127-138]. Currently, Food and Drug Association (FDA) requires demonstration of a minimum total of 6 LRVs (Log Reduction Value) of viral clearance using 2 orthogonal techniques with a minimum of 4 LRVs from one of the methods [Shukla & Aranha (2015) Pharmaceutical Bioprocessing 3(2):127-138]. However, VC studies, typically conducted at third party sites, tend to have long turnaround time of 4 to 7 months. Thus delay or failure to comply with VC validation requirements could hinder development, scale-up and commercialization of particularly for newer modality therapeutics, atypical process conditions or newer virus inactivation (VI) agents [Sipple et al. (2019) Biotechnology progress 35(5): e2850]. We propose an efficient and comprehensive strategy to screen VI conditions to reduce risks of VC validation failure to ensure expedited drug delivery to clinical trial patients.

While VC from low pH and chromatography or filtration-based operations has been extensively summarized, detergent-mediated VI has been studied typically for Triton X-100 [Cipriano et al., Effectiveness of various processing steps for viral clearance of therapeutic proteins: database analyses of commonly used steps, in Therapeutic Proteins. 2012, Springer. p. 277-292; Brorson et al. (2003) Biotechnology and Bioengineering 82(3):321-329; Ma & Roush (2016) PDA Journal of Pharmaceutical Science and Technology 70(5):410; Jin et al. Protein aggregation and mitigation strategy in low pH viral inactivation for monoclonal antibody purification, in MAbs. 2019. Taylor & Francis].

Traditionally, Triton X-100 (C₁₄H₂₂O(C₂H₄O)n) has been used for VI in the pharmaceutical industry. It is a nonionic surfactant that has a hydrophilic polyethylene oxide chain and an aromatic hydrocarbon group of 1,4-(1,1,3,3-tetramethylbutyl) phenol. However, through stepwise removal of ethylene oxide, Triton X-100 degrades into 4-tert-octylphenol, which is an endocrine disruptor with adverse estrogenic effect on aquatic species, animals and humans. [Farsang et al. (2019) Molecules 24(7):1223; Kano & Ishimura (1995) Journal of the Chemical Society, Perkin Transactions 2(8):1655-1660] As such, European Chemicals Agency (ECHA) has deemed Triton X-100 to be a substance of very high concern (SVHC) and has mandated its replacement in all manufacturing processes. [Conley et al. (2017) Biotechnology and Bioengineering 114(4):813-820; U.S. Pat. No. 10,611,795; Adopted opinions and previous consultations on applications for authorisation. echa.europa.eu/applications-for-authorisation-previous-consultations/-/substance-rev/23826/term 2019] Thus, there has been an industry-wide initiative to replace Triton-X100 in manufacturing processes. Alternatives to Triton X-100 include pH neutral arginine buffer for VI of X-MuLV (Xenotropic Murine Leukemia Virus) and PRV (Pseudorabies virus) [McCue et al. (2014) Biotechnology Progress 30(1):108-112], caprylate for VI of HSV-1 (Herpes Simplex Virus type 1) and Sindbis virus [Lundblad & Seng (1991) Vox Sanguinis 60(2):75-81] and Simulsol SL 11W for VI of X-MuLV [Luo et al. (2020) Identification and Characterization of a Triton X-100 Replacement for Virus Inactivation. Biotechnology Progress 36.6: e3036]. Arginine and LDAO have also been used in protein A wash buffers for X-MuLV clearance [Bolton et al. (2015) Biotechnology Progress 31(2):406-413].

With the advent of continuous low pH VI, there are concerns over achieving adequate VC without adverse impact on product quality attributes. Since continuous low-pH VI achieves inactivation through mimicking plug flow regime in a tubular reactor, the ideal residence time is critical to ensure adequate VI while avoiding degradation products arising from prolonged or localized exposure to low pH solution conditions. [Jungbauer (2019) Biotechnology Journal 14(2):1800278; Konstantinov & Cooney (2015) Journal of Pharmaceutical Sciences 104(3):813-820; Gillespie et al. (2019) Biotechnology Journal 14(2):1700718] Detergent-mediated VI could thus provide an orthogonal means of VC for molecules sensitive to low pH and for next generation continuous bioprocessing with minimal risk of product degradation.

In addition to removing viruses, a critical criterion for detergent selection is to ensure complete clearance of detergents with minimal impact on product quality. [Conley et al. (2017) Biotechnology and Bioengineering 114(4):813-820] To this effect, we assessed the impact of detergents on several quality attributes such as potency, charge variants, oxidation, deamidation, glycosylation and aggregation. Drug efficacy or potency for the purified drug substance (DS) in the presence of different detergents was evaluated as a measure of product quality. [Rowshanravan et al. (2018) Blood 131(1):58-67] The impact of the detergents on the charge variants of the proteins arising from specific detergent-protein interaction and protein unfolding or aggregation was also assessed. Any protein unfolding, denaturation or aggregation caused by detergents could solvent expose oxidation- or deamidation-prone amino acids of proteins forming products such as iso-aspartic acid thus increasing the risk of immunogenic responses in humans. [Manning et al. (2010) Pharmaceutical Research 27(4):544-575; Jenkins et al. (2008) Molecular Biotechnology 39(2):113-118] Methionine oxidation and asparagine or glutamine deamidation of DS was thus also evaluated. Sialic acid, arising from glycosylation of proteins, minimizes protein self-association by shielding aggregation-prone, e.g., hydrophobic sites on protein. [Jing et al. (2010) Biotechnology and Bioengineering 107(3):488-496; Sinclair & Elliott (2005) Journal of Pharmaceutical Sciences 94(8):1626-1635; Soli & Griebenow (2009) Journal of Pharmaceutical Sciences 98(4):1223-1245] Tringali et al. showed that detergents may alter activity of the enzyme, sialidase thereby impacting the sialic acid content and hence solubility and stability of proteins. [Jing et al. (2010) Biotechnology and Bioengineering 107(3):488-496; Tringali et al. (2004) Journal of Biological Chemistry 279(5):3169-3179] We explored the sialic acid content in the form of NANA (N-acetylneuraminic acid) or NGNA (N-glycolylneuraminic acid) in the protein A eluates of detergent-spiked clarified harvest. The amphipathic nature of detergent molecules results in interaction of detergents with both the hydrophobic and hydrophilic regions of the proteins leading to protein aggregation or HMW (High Molecular Weight species) formation. Protein unfolding was modeled by the Lumry-Eyring framework, beginning with first order reversible protein unfolding (RLS=rate limiting step) followed by higher order aggregation. [Shukla et al. (2007) Journal of Chromatography A 1171(1-2):22-28; Kendrick et al. (1998) Proceedings of the National Academy of Sciences USA 95(24):14142-14146.]

We looked into the process-mediated detergent and impurity clearance of the most promising VI agents through protein A purification Next, we screened non-ionic and zwitterionic detergents of low biotoxicity as VI agents for inactivation of three different lipid-enveloped viruses in two different therapeutic modalities of CHO cell origin. The kinetic and/or mechanistic understanding of factors driving inactivation and protein instability can offer valuable guidance to efficiently screen VI conditions without compromising on product quality and impurity clearance.

### 2. Materials and Methods

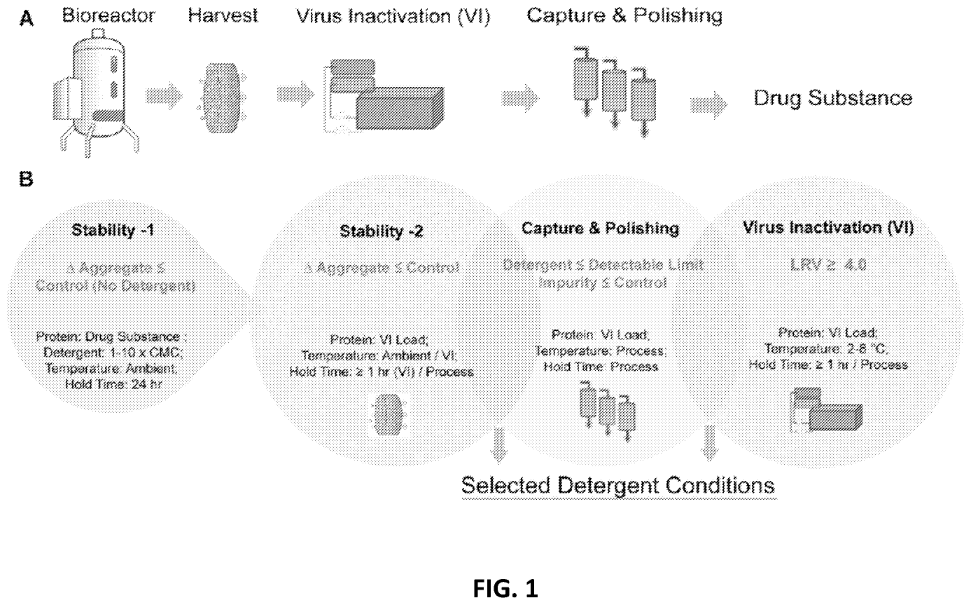

The screening study was divided into three stages-stability, column-based impurity clearance followed by VI as seen in FIG. 1, panel A. The stability study was divided into two segments. A first initial screening involved assessing aggregate formation for protein DS at concentrations comparable to process conditions representative of VI. The DS was treated with high and low concentrations of detergent over a 24 hour period at room temperature as worst case for stability. The detergent conditions that showed levels of aggregate formation less than or comparable to control (no detergent) or less than 2% HMW formation over a 24 hour period were carried over for a more comprehensive stability screening in alignment with the process developed. The protein was incubated at the highest temperature, i.e., room temperature and longest duration that conformed to the acceptable operating range as a worst case for stability. Thus, for the fusion proteins tested, the respective harvest pool was spiked with detergent, protein A purified and the eluate evaluated for product quality. Quality attributes tested included aggregation, potency, charge variance, oxidation-deamidation and glycosylation. In addition, the detergent and impurity content of the eluate including host cell protein, residual protein A and DNA were evaluated and compared with the control. Conditions that showed promising clearance were carried over for the final stage, i.e., VI study at a third party testing site at lowest temperature of 2-8° C. and shortest duration of 1 hour as a worst case for VI. The stability screening was conducted in two stages—a preliminary screening with DS for aggregate formation and a final screening into process representative VI load. The preliminary screening involved detergent spiking into DS to rule out conditions that could lead to pronounced aggregate formation. The final process-representative VI stability screening involved detergent spiking into VI load, chromatographic capture or polishing and assessment of the VI pool generated for product quality attributes. Following stability, the detergent and impurity clearance by the chromatographic step following VI was tested. For the fusion proteins tested, detergent was spiked into the harvested cell culture fluid, i.e., the VI load followed by protein A purification. The protein A eluate was then assessed for product quality as well as detergent and impurity clearance. The final step in the VI screening involved VI study with Bio Safety Level-2 (BSL2) viruses at a third party testing site.

The Fus1 in the present example is abatacept, whereas the Fus2 protein is belatacept.

### 2.1 List of Detergents

The DS at high and low concentrations as well as the harvest for both the molecules was spiked with detergents (TABLE 1) and incubated at room temperature.

TABLE 1
List of detergents

| | | Catalogue |
|---|---|---|
| Detergent Name | Vendor | Information |
| n-Octyl-β-D-Glucopyranoside (OG) | Anatrace | 0311S |
| Octyl β-D-1-thioglucopyranoside | MilliporeSigma | O6004 |
| (OTG) | | |
| Nonyl β-D-glucopyranoside (NG) | MilliporeSigma | N7507 |
| n-Dodecyl-β-D-Maltopyranoside | Anatrace | D310S |
| (DDM) | | |
| n-Decyl-β-D-Maltopyranoside (DM) | Anatrace | D322S |
| 5-Cyclohexylpentyl β-D-maltoside | MilliporeSigma | 96193 |
| Trehalose 6-dodecanoate (Tre-6-DTT) | MilliporeSigma | BCBZ4669 |
| Lauryl-6-Trehaloside | Anatrace | 6DDTre |
| Lauryldimethylamine-oxide (LDAO) | Anatrace | D360 |
| EcoSurf ™ EH9 | MilliporeSigma | STS0006 |
| Zwittergent R 3-12 | MilliporeSigma | 693015 |
| Tergitol L-64 | DOW | 77605 |
| Triton CG-650 | DOW | 184667 |
| Triton CG-110 | MilliporeSigma | STS0005 |
| PS80 (NOF) | NOF | Polysorbate |
| | | 80(HX2) |
| PS80 (CRODA) | J. T. Baker | 4117, 4500 |
| Taurocholic acid sodium salt hydrate | MilliporeSigma | T4009 |
| (NaTC) | | |
| Triton X-100 | MilliporeSigma | X100-500ML |

For both Fus1 and Fus2, the VI step is after pH neutralization of harvested cell culture fluid and prior to protein A purification. Thus, for the impact of detergent to be representative of the process, the harvest material was incubated with the key detergents at room temperature over extended an duration of 57 hours (Fus1) and 38 hours (Fus2). The longest hold duration was representative of processing conditions as worst case for stability.

### 2.2. Quality Attributes

For stability screening, mAb1 DS was spiked with known amounts of detergents including Triton X-100 for a minimum of one hour at 2-8° C. and tested for product quality attributes—a control with no detergent was used for comparison. The product quality attributes studies included HMW (High Molecular Weight) species, potency and charge distribution profile.

For the fusion proteins, the DS of Fus1 and Fus2 at high and low protein concentrations was spiked with detergent and incubated at room temperature for up to 24 hours. For the spiking study to be process representative of worst case for stability and to demonstrate detergent clearance, the harvested clarified cell culture fluid of the two fusion proteins were spiked and protein A purified following room temperature hold times of up to 55 hours for Fus1 and 38 hours for Fus2 respectively. The protein A eluates were then tested for different quality attributes. Multiple techniques were used to analyze the different molecular weight species generated in the protein A eluate of the detergent-spiked harvest. The methods include SEC (size exclusion chromatograph), higher resolution tandem SEC and NR SDS-PAGE (Non reduced sodium dodecyl sulfate polyacrylamide gel electrophoresis) which are detailed below along with other characterization techniques.

### 2.2.1 Size Exclusion Chromatography

Size exclusion chromatography (SEC) was performed in a Waters HPLC Alliance 2695 System using TSKgel G3000SW_{XL} Column (Tosoh Bioscience, Catalogue no. 085430) with guard column (Tosoh Bioscience, Catalogue no. 08541) in line. A mobile phase of 0.2 M Sodium phosphate, monobasic, 0.9% NaCl, pH 7.0 was used with 20 μL injection volumes at 1 to 10 mg/mL target protein concentrations with a flow rate of 1 mL/minute. Tandem SEC was performed with 50 μL injection volumes with a flow rate of 0.5 mL/minute using 6 TSKgel G3000SW_{XL} Column using 0.2 M KH₂PO₄, 0.9% NaCl, pH 6.8 as mobile phase.

### 2.2.2 NR SDS-PAGE

Non-reduced sodium dodecyl sulfate polyacrylamide gel electrophoresis (NR SDS-PAGE) was run using 4-20% Tris-Glycine Mini Gels, WEDGEWELL™ Format 12-well (Invitrogen, Catalogue no.: XP04202BOX) and 1× Tris-Glycine SDS Running Buffer and stained with Coomassie blue. GS-900 Densitometer with Image Lab Software (Bio-Rad, Catalogue no.: SFAWBA10464) was used to analyze the gels to identify different molecular weight species in the protein sample.

### 2.2.3 Potency

The potency was determined by measuring the binding efficacy of the CTLA-4 (cytotoxic T-lymphocyte-associated protein 4) domain of the fusion proteins against the complementary binding domain of membrane protein B7.1 Ig, typically found on activated antigen-presenting cells (APC) [23]. Drug efficacy for the purified DS was evaluated using a Surface Plasmon Resonance assay; the binding efficacy of the CTLA-4 domain of Fus1 and Fus2 against a high concentration of the peripheral membrane protein B7.1 Ig was examined. Due to the influence of impurities on the binding efficacy, only DS was tested. Low DS concentrations of 3 g/L were tested to show comparable potency to the reference material (RM). The acceptable limit for potency being 70 to 130% for Fus1 and 75 to 125% for Fus2.

### 2.2.4 Sialic Acid Content

Sialic acids are neuraminic acids modified by the addition of an acetyl group (N-acetylneuraminic acid/NANA) or a glycol group (N-glycolylneuraminic acid/NGNA) .Sialic acid contents are reported as normalized molar ratios, which are the total moles of NANA and NGNA per mole of recombinant protein. The protein concentrations were determined by UV absorbance at 280 nm. Sialic acid content was determined by partial acidic hydrolysis using sulfuric acid at 0.1 N final concentration at 80° C. for 1 hour followed by reversed phase HPLC using Rezex Monosaccharide RHM HPLC column (Phenomenex, Catalog No. OOH-0132-KO with respective guard column (Phenomenex, Catalog No. 03B-0132-KO). Elution was conducted with 5 mM Sulfuric acid at 0.6 mL/min and 40° C.

### 2.2.5 Oxidation and Deamidation

The protein sample was denatured in denaturation buffer (8 M guanidine, 50 mM TRIS, pH 8.0) and the cystine disulfide bridges were reduced with dithiothreitol (200 mM DTT) followed by S-alkylation with iodoacetamide (400 mM IAM). The denatured, reduced protein was buffer-exchanged (50 mM TRIS, 10 mM CaCl₂), pH 7.6) prior to digestion with trypsin. The resulting digested mixture was then analyzed by reverse-phase ultra-performance liquid chromatography (RP-UPLC) using UPLC BEH C18 Column, 1.7 M, 130 angstrom, 2.1×100 mm (Waters, Catalog no. 186002352) with Mobile Phase A (0.1% TFA, 50 mM Methionine in HPLC Grade Water) and Mobile Phase B (0.1% TFA, 50 mM Methionine in 80% ACN and 20% HPLC grade water). Protein detection at 215 nm absorbance and fluorescent excitation/emission of 275 nm/303 nm and 280 nm/348 nm was used to quantify relative levels of oxidation and deamidation respectively.

2.2.6 Imaged Capillary Isoelectric Focusing (iCIEF)

An imaged capillary isoelectric focusing (iCIEF) method is a charge-based separation of different protein isoforms by isoelectric point (pI). Protein samples at final concentration of 1.0 mg/mL were injected by an autosampler into a capillary cartridge within the instrument. The sample was prefocused for 1 minute at 1500 V and then focused for 9 minutes at 3000 V. Sample migration was captured by a CCD camera that took a UV light absorption image, and the peaks were analyzed using the associated software to categorize the peaks into different pI marker regions.

### 2.3 Detergent and Impurity Clearance

### 2.3.1 Detergent Clearance

A Charged Aerosol Detector (CAD) based Reversed Phase High-Performance Liquid Chromatography (RP-HPLC) method was used for the detection of the detergents, OG and DDM in protein A load, flow through, and eluate of Fus1 and Fus2 harvested cell culture fluid spiked with respective detergents. XBridge BEH C4 Column (Waters, Catalog no 186004499) was used to separate the detergent from the protein using 0.02% formic acid in water/methanol as mobile phase A/B, respectively. Charged aerosol detector (Corona Ultra RS or equivalent) based detection of detergent was conducted. The areas under the peaks were plotted against the nominal detergent concentrations using a quadratic equation with the limit of quantitation being 0.003% for both OG and DDM.

### 2.3.2 DNA Clearance

Residual CHO cell DNA was quantified by qPCR assay using the TaqMan probe with forward and reverse primers flanking specific repetitive sequence of CHO cell genome. The fluorescent receptor was at the 5′-end and the quencher in the 3′ end quenching the receptor fluorescence. With amplification, the exonuclease activity of the Taq polymerase reaction released the reporter dye leading to a fluorescent signal. The number of amplification cycles needed to reach a threshold fluorescence was inversely proportional to DNA content in original sample. A standard curve of cycle number with reference CHO cell DNA was used to quantify the DNA in the unknown sample.

### 2.3.3 HCP Clearance

Enzyme-Linked Immunosorbent Assay (ELISA) was used for quantitating the level of CHO host cell protein in protein A eluates.

### 2.3.4 Residual Protein A Clearance

Enzyme-Linked Immunosorbent Assay (ELISA) was used for quantitating the level of residual protein A in the eluates. The anti-protein A coated microtiter plate was incubated with the sample and then treated with biotinylated anti-protein A. The plate was treated with streptavidin conjugated peroxidase and TMB (3,3′,5,5′-Tetramethylbenzidine) was used to generate a colorimetric response. The reaction was quenched with an acidic solution and the absorbance was measured at 450 nm. A calibration curve of absorbance against known protein A standards was generated to quantify the protein A content.

### 2.4 Modeling Methodology

The 3D tertiary structure of proteins Fus1 and Fus2 were obtained using the homology modeling protocol within BIOVIA Discovery Studio software (Discovery Studio Modeling Environment, Release 4.1, San Diego: BIOVIA Software Inc., 2014). We then used the Spatial-Aggregation-Propensity (SAP) model to determine the hydrophobic patches on the protein surface. [Chennamsetty et al. (2009) Proceedings of the National Academy of Sciences USA 106(29):11937-11942]. The SAP model was applied to the homology-modelled structure using the BIOVIA Discovery Studio software. The proteins were then colored based on the SAP values for each atom. A positive SAP value was given a red color, SAP values near zero were white and negative SAP values were blue color. Thus, the red colored regions represented the positive SAP values, which are hydrophobic patches on the protein surface. The overall SAP score for Fus1 and Fus2 was calculated by summing the SAP values that were positive for each atom.

### 2.5 Statistical Analysis

SAS-JMP Version 13.1.0 was used to perform statistical analysis for all experiments in this report. A backwards stepwise regression, with a p-value of 0.05 was constructed for the extent and rate of HMW formation starting with the main effects of all parameters that were believed to impact aggregation: type of detergent, protein or molecule, detergent concentration, protein concentration, and initial HMW %. The final model was determined by eliminating nonsignificant effects (p-value >0.05) from highest p-value to lowest. The coefficient of determination (R²), was used to explain the amount of overall variation explained by the model. The adjusted R², adjusted for the number of parameters in the model, was used in tandem with R² to assess whether or not the models were overfit. The predicted R2 was used to test the robustness of each model, and a difference between the adjusted R2 and the predicted R2 of approximately 0.2 or less indicated the model was robust. This statistics-based approach assumed that the model residuals were independently and normally distributed with a mean of zero and constant variance. Residuals were visually inspected and analyzed using a residuals plot and a Normal Q-Q plot to confirm these assumptions for each fitted model. The studentized residuals plot was used to identify potential outliers. A Cook's Distance was obtained to help determine if identified outliers were influential. An influential outlier was either transformed to meet model assumptions or removed in fitting the model if necessary. The Box-Cox Y transformation test was performed to identify the best transformation for fitting the model if there were identified influential outliers or other model assumptions were not satisfied

### 2.6 Virus Inactivation

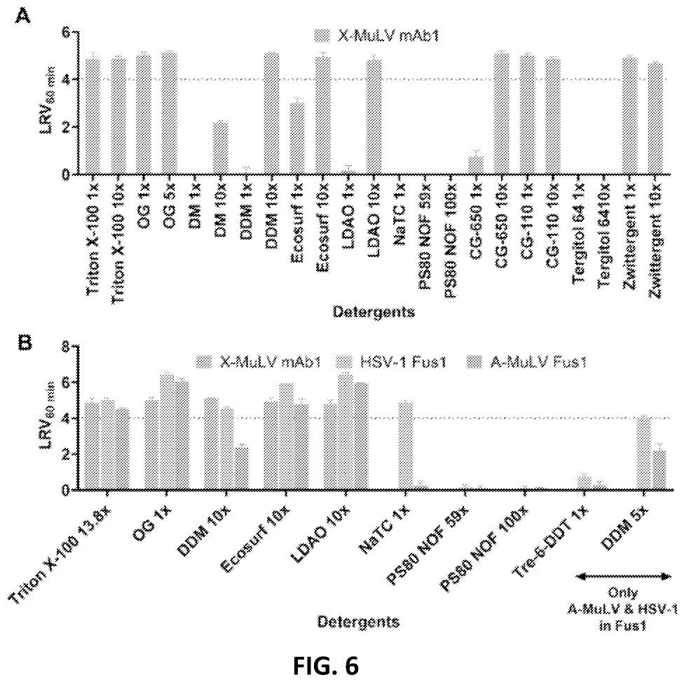

Three lipid-enveloped viruses X-MuLV, HSV-1 and A-MuLV (Amphotropic Murine Leukemia Virus) were investigated for VI in the cell culture fluid of a monoclonal antibody (mAb1) and a fusion protein (Fus1) for a range of detergents and detergent concentrations (FIG. 6). Protein concentration and buffer matrix were dictated by that in the harvest for the specific therapeutic and process. A temperature of 2-8° C. was used as a worst case for inactivation and incubation times of 0, 5 and 60 minutes were used to explore the kinetics of inactivation. Viral clearance was measured in units of Log Reduction Value (LRV) which was calculated from pfu (Plaque Forming Units) as follows

$$LRV = - {log}_{10} \frac{{[ Virus in pfu ]}_{P r o d u c t p o o l}}{{[ Virus in pfu ]}_{L o a d / F e e d}} .$$

Virus inactivation studies were conducted for X-MuLV in mAb1 harvest, HSV-1 and A-MuLV in Fus1 harvest at 0, 5 and 60 minutes at 2-8° C. The inactivation studies for each condition were conducted in duplicates with results varying within 0.5 LRV. The lower of the two LRVS was plotted in FIG. 6 to remain conservative regarding the extent of inactivation. The error bars represent the assay variability.

### 3. Results and Discussion

Our results provided a detailed strategy to screen conditions for VI in three stages. The first stage of VI evaluation constituted verifying the therapeutic product quality in presence of the detergent in the purified product as well as the protein A eluate of the detergent-spiked clarified harvest. This step was followed by demonstration of effective detergent and impurity clearance through column-based capture step and finally, testing the detergent efficacy for viral clearance (FIG. 1). Conducting VI as a final stage ruled out conditions that did not meet the stability and impurity clearance criteria.

Reducing the number of test conditions entering into a VC study lead to significant savings in cost and turnaround time. Details of the strategy are stated in the Materials and Methods section.

### 3.1 Therapeutic Product Quality

A change in product quality such as protein aggregation can lead to loss of drug potency or immunogenic response in patients [Liu et al. Acid-induced aggregation propensity of nivolumab is dependent on the Fc. in MAbs. 2016. Taylor & Francis; Moussa et al. (2016) Journal of Pharmaceutical Sciences 105(2):417-430]. Stability of proteins during detergent-mediated VI is influenced by several factors which include temperature, duration of incubation, detergent concentration, type of protein, its concentration and the solution matrix of inactivation. [Brorson et al. (2003) Biotechnology and Bioengineering 82(3):321-329; Ma & Roush (2016) PDA Journal of Pharmaceutical Science and Technology 70(5):410; Conley et al. (2017) Biotechnology and Bioengineering 114(4):813-820]. We explored the stability of proteins for a number of these factors; the study details are provided in Materials and Methods and Supplementary Information, infra.

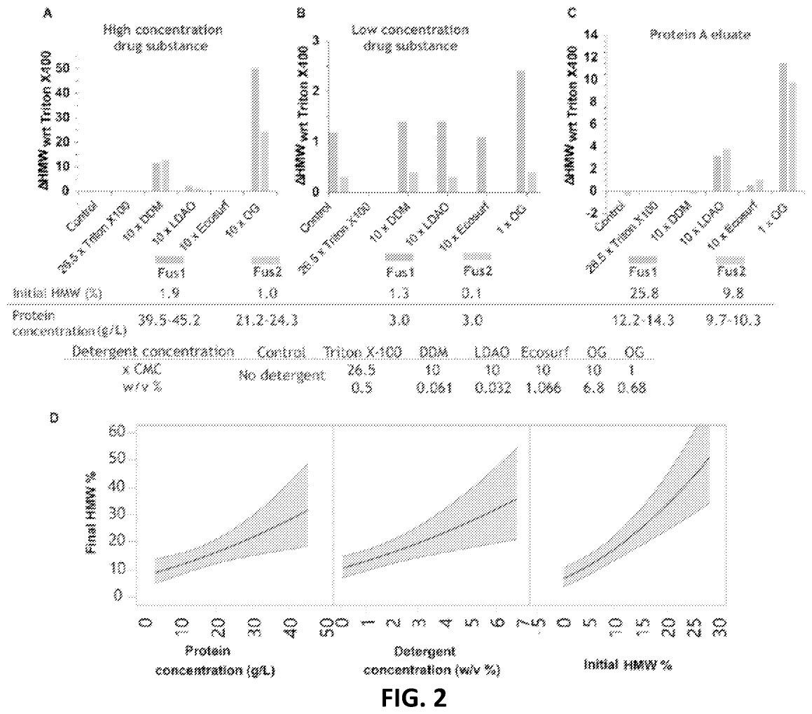

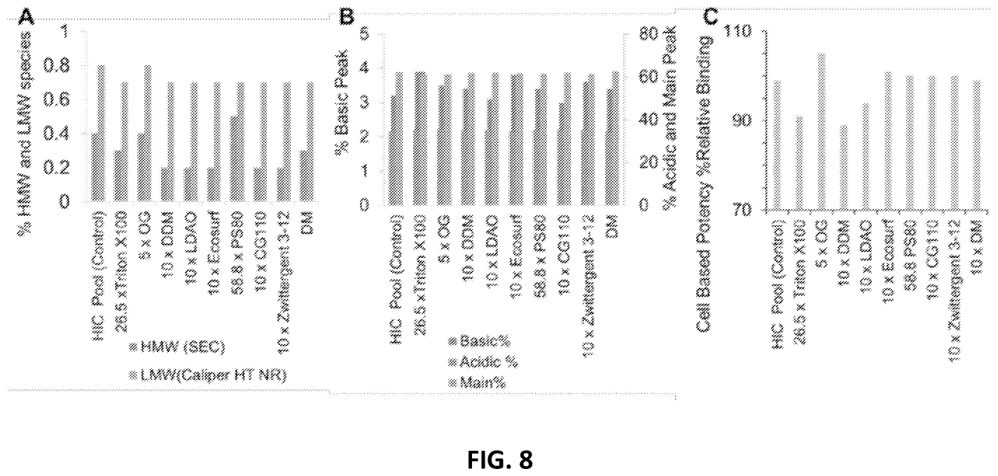

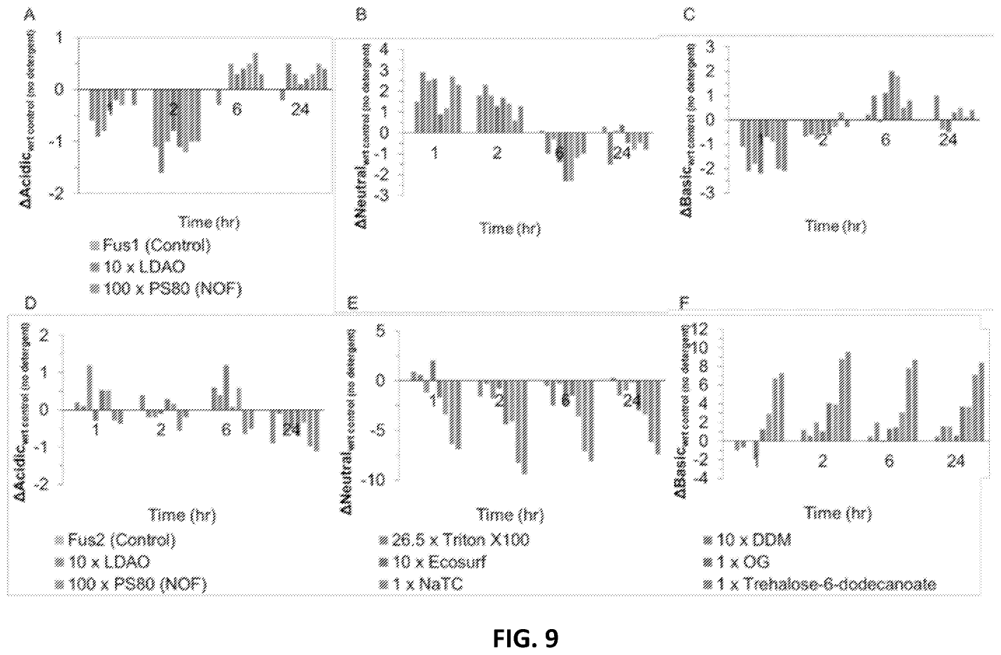

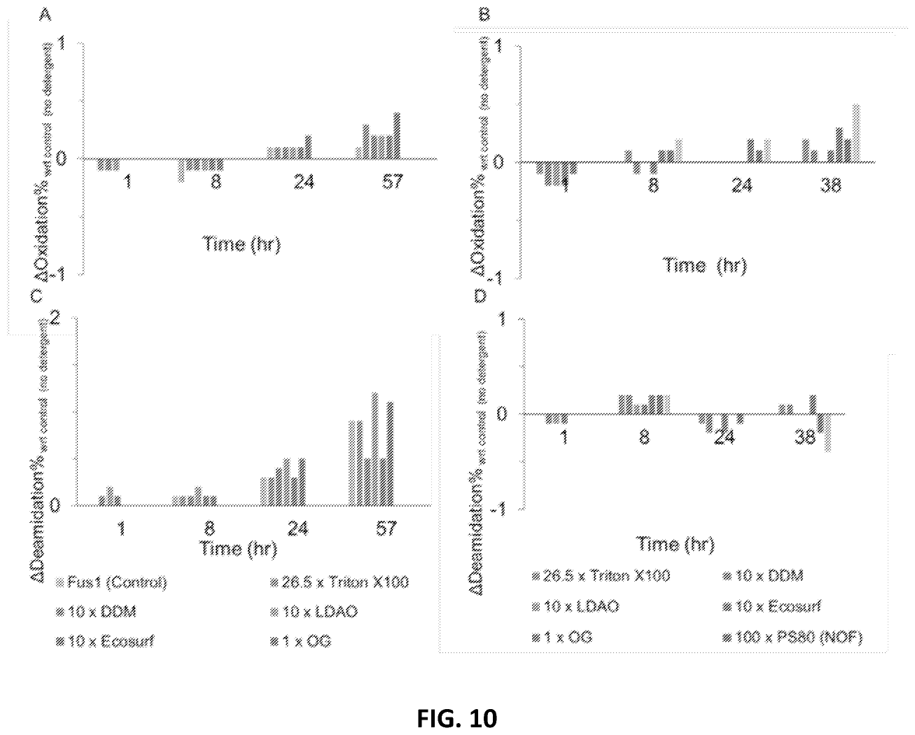

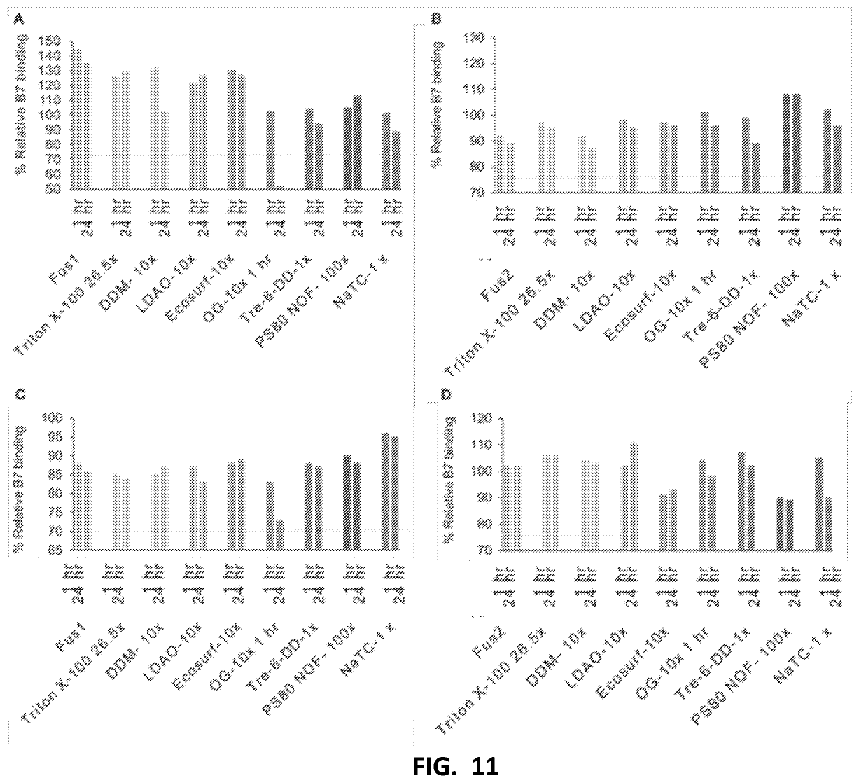

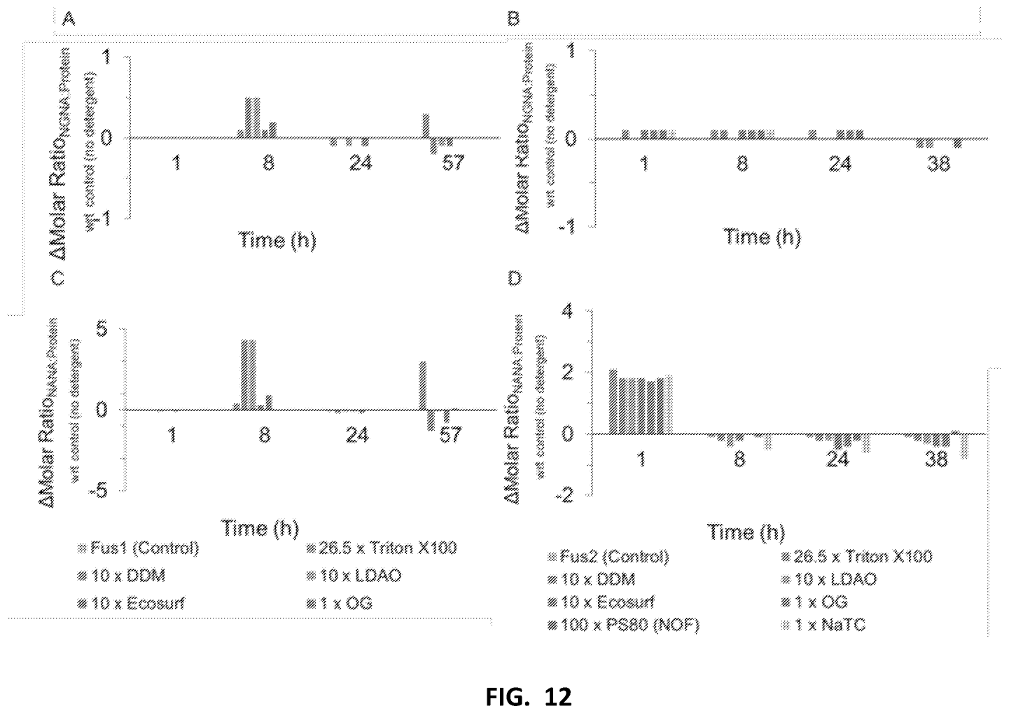

For mAb1 drug substance (DS), no significant impact was observed on any of the product quality attributes (FIG. 8). Unlike the mAb1 (pI of ˜8.6), owing to the pH-sensitive nature of the fusion proteins (pI of ˜5.0), Fus1 and Fus2 could not be subjected to low pH VI. As such, an extensive stability screening was conducted on Fus1 and Fus2 as detailed in the Material and Methods. Relatively little change was observed in the charge profile for both molecules as would be expected with the use of non-ionic and zwitterionic detergents in this study (FIG. 9). No significant difference in these oxidation and deamidation profiles were observed for most cases, except OG at 1×CMC which showed higher oxidation for both Fus1 and Fus2 harvest (FIG. 10). All conditions showed comparable potency to Fus1 and Fus2 reference material respectively. Higher concentration DS was worst case for potency, particularly for OG at 10×CMC in Fus1, which showed less than 60% potency at 24 hours (FIG. 11). The greater decline in potency for Fus1 versus Fus2 could be attributed to greater extent of HMW formation for OG in Fus1 versus OG in Fus2 (FIG. 2). Glycosylation in the form of sialic acid content is a critical product quality parameter. While NANA is produced by humans, NGNA may have immunogenic response [Moussa et al. (2016) Journal of Pharmaceutical Sciences 105(2):417-430; Suriano et al. (2009) Glycobiology 19(12):1427-1435] No significant variation was observed in NANA or NGNA sialic acid levels for the different detergents in Fus1 (FIG. 12).

### 3.1.1 Aggregation

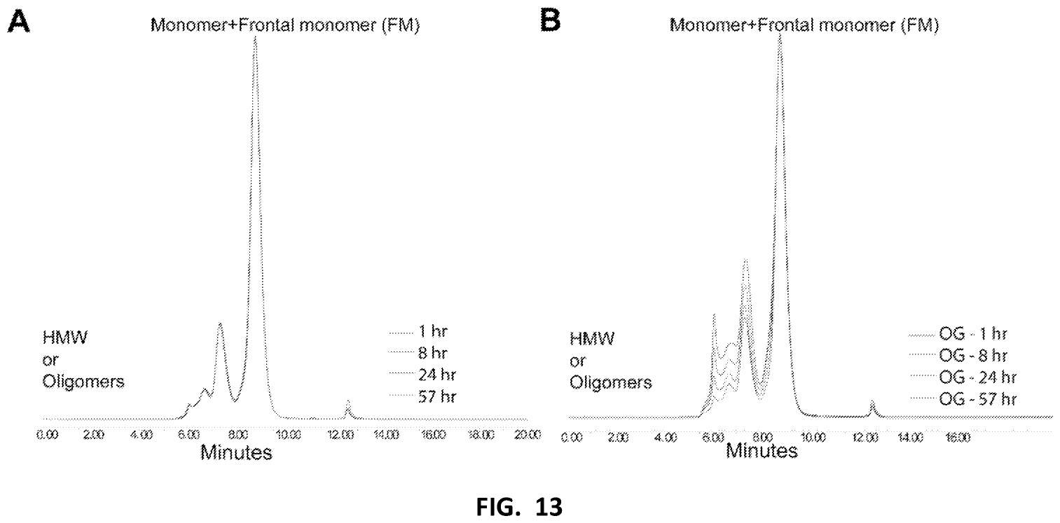

Initial self-association of native or folded protein can lead to formation of reversible aggregate while refolding of unfolded or non-native protein through hydrophobic interactions can lead to formation of irreversible aggregates [Shukla et al. (2007) Journal of Chromatography A 1171(1-2):22-28; Kendrick et al. (1998) Proceedings of the National Academy of Sciences USA 95(24):14142-14146]. Detergents could interact and unfold proteins exposing their hydrophobic domains; leading to aggregation through hydrophobic interaction [Feroz et al. (2018) Analyst 143(6):1378-1386; Tulumello et al. (2012) Biochimica et Biophysica Acta-Biomembranes 1818(5):1351-1358]. The confirmation of the irreversible aggregates [Shukla et al. (2007) Journal of Chromatography A 1171(1-2):22-28; Kendrick et al. (1998) Proceedings of the National Academy of Sciences USA 95(24):14142-14146] thus formed was obtained by alternative techniques which included SDS-PAGE gels, tandem SEC (data not shown) and SEC [Boyd et al. Isolation and characterization of a monoclonal antibody containing an extra heavy-light chain Fab arm. in MAbs. 2018. Taylor & Francis]. SEC profiles showed HMW increase and monomeric protein decrease over time (FIG. 13).

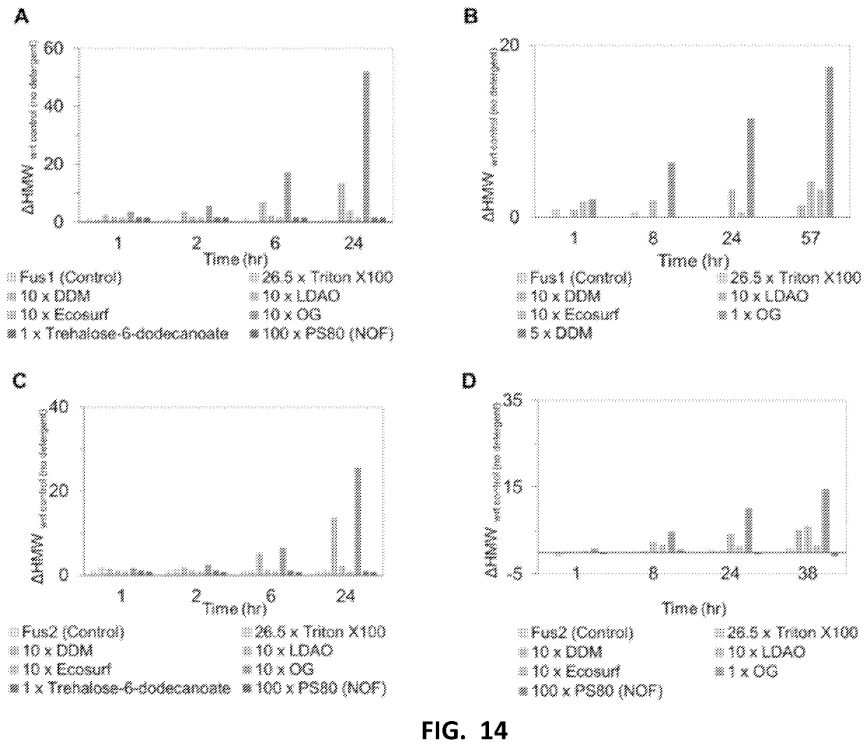

SEC analysis was conducted across several detergents for Fus1 and Fus2 (FIG. 14) and data for detergents carried forward for VI is summarized in FIG. 2. The HMW generated over a 24 hour hold was modeled against several process conditions which include protein type, its concentration, solution matrix, initial HMW content, detergent type and its concentration. Our model with a strong correlation of R2=0.91, ANOVA p value=0.0002, indicated that the factors critical in determining extent of aggregation were detergent concentration and protein concentration as well as initial HMW levels (FIG. 2, panel D). HMW formation increased in instances of higher initial HMW, higher protein concentration as well as higher detergent concentration.

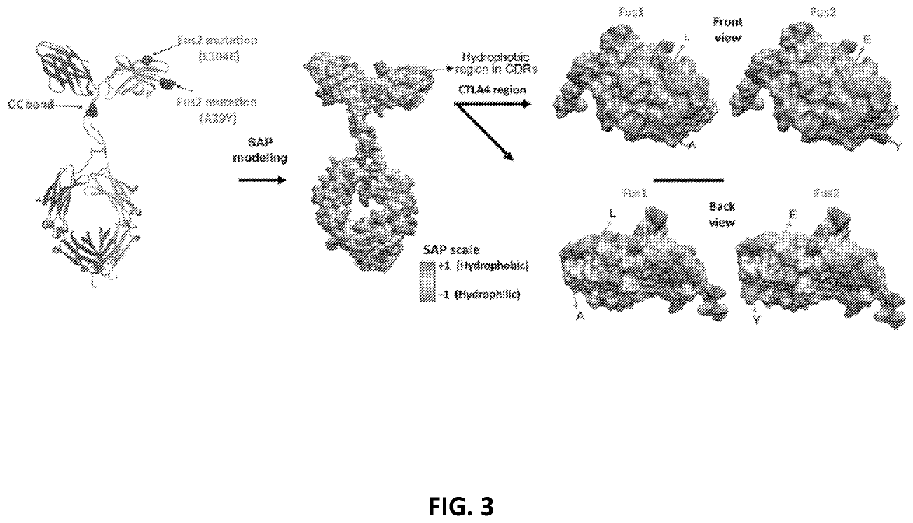

Although comparable HMW formation was observed in protein A eluate obtained from detergent-spiked clarified cell culture harvest at 2 to 3 g/L, Fus1 had higher initial HMW than Fus2 (FIG. 2, panel C). At comparable DS concentration of 3 g/L, greater HMW formation was observed for Fus1 in comparison to Fus2 (FIG. 2, panel A). While the different HMW levels could be impacted by the solution matrix, e.g., upstream media conditions, a key contributor to stability appeared to be the protein structural difference. [Chennamsetty et al. (2009) Proceedings of the National Academy of Sciences USA 106(29):11937-11942] We also performed SAP (Spatial-Aggregation-Propensity) modeling on both molecules to identify the possible impact of molecule structure on extent of aggregation. A SAP model identified hydrophobic patches on the protein surface prone to aggregation or binding and also gives a score for the overall hydrophobicity of the molecule. [Chennamsetty et al. (2009) Proceedings of the National Academy of Sciences USA 106(29):11937-11942] As shown in FIG. 3, Fus1 differs from Fus2 by two point mutations which explained the different hydrophobicities of the two molecules. [Bluestone et al. (2006) Immunity 24(3):233-238] The SAP score for Fus1 was 115.4 whereas for Fus2 it was 110.3. The mutation of the more hydrophobic amino acid, L (leucine) to E (glutamic acid) disrupted the large hydrophobic patch in Fus1 into a smaller hydrophobic patch in Fus2. The mutation of the smaller hydrophobic amino acid, A (Alanine) to larger hydrophobic amino acid, Y (Tyrosine) in Fus2 introduced a new but significantly smaller hydrophobic patch in comparison to that in Fus1. The higher SAP score for Fus1 indicated greater hydrophobicity and explained the higher aggregation tendency of Fus1 at comparable protein and detergent concentrations than Fus2 (FIG. 2, panel B).

### 3.1.2 Kinetics of Aggregation

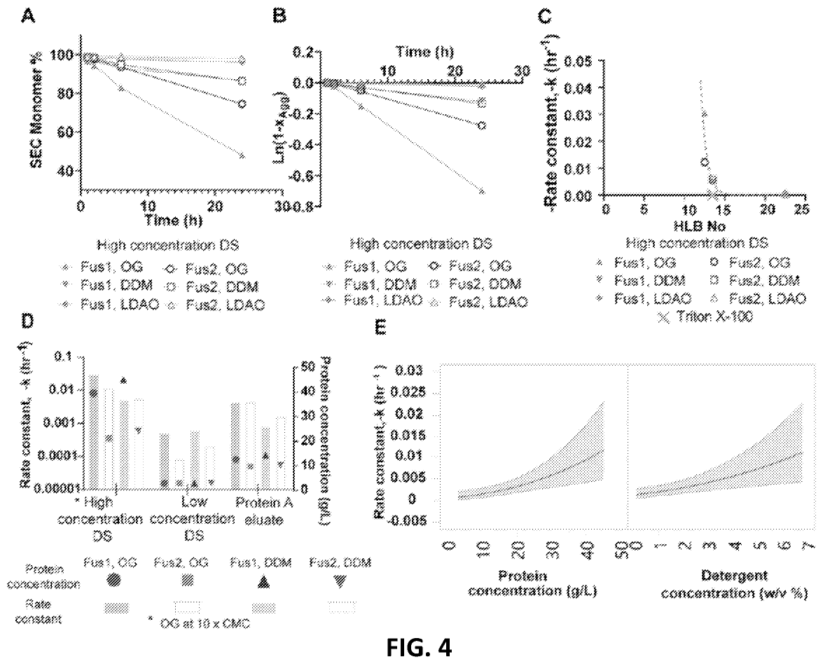

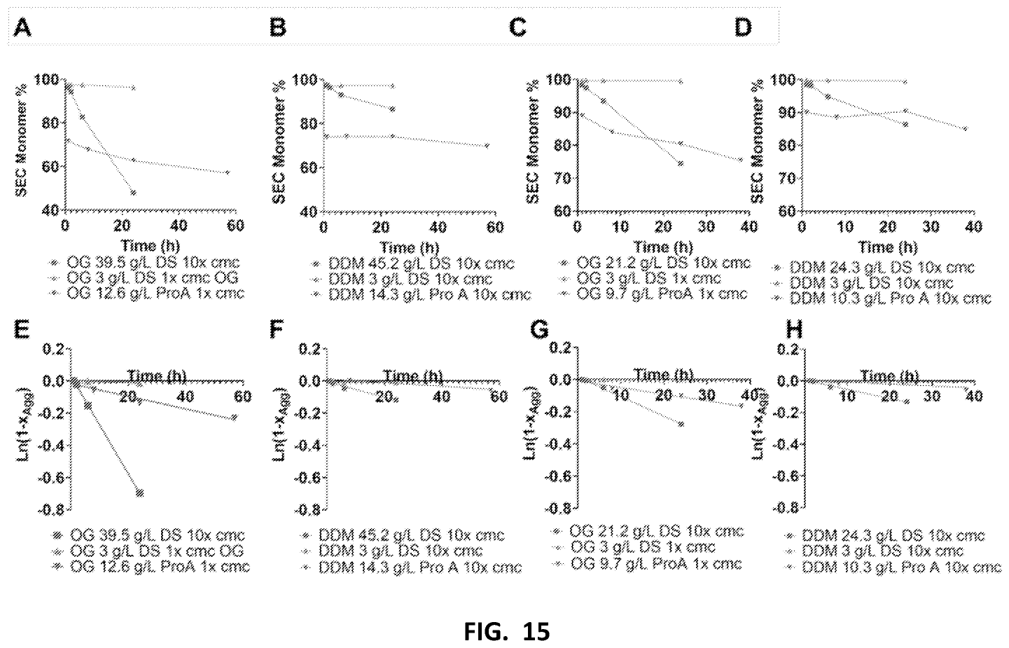

HMW formation showed first order kinetics for OG, DDM and LDAO in high concentration DS and also for OG and DDM in harvested cell culture fluid (FIG. 4, panels A-C; and FIG. 15). Given N is the monomer species, No is the initial monomer % at time t=0, aggregation follows first order kinetics with k₁ being the rate constant of aggregation as shown in Equation (1) (Derivation in Supplemental Information).

$$ln \frac{[ N ]}{[ {N}_{o ]}} = - {k}_{1} t \qquad ( 1 )$$

The linear trend of natural logarithm of monomer concentration over time, even at high conversion to HMW, also indicated that the rate-limiting step in the process was not the protein-protein collision but rather the preceding step of conversion of the monomer (N) to transient unfolded state as seen in FIG. 4, panel B. For high concentration DS, OG at 10×CMC exhibited higher rate constant for aggregation in both Fus1 and Fus2 than DDM at 10×CMC. Assuming that the protein unfolding was initiated by protein-detergent interaction, the kinetics or extent of HMW formation was tied to the hydrophobicity of the micelles. One parameter for the measure of hydrophobicity is the hydrophilic lipophilic balance number (HLB)—the lower the hydrophobicity, the greater the HLB. Intuitively, OG has the smallest head-group, followed by DDM and consequently OG has the tightest lipid packing rendering it the greatest hydrophobicity. LDAO has a zwitterionic head group, which gives its micelles greater solubility and thus higher HLB value. Fus1 showed a greater extent of HMW formation than Fus2 for OG [Feroz et al. (2018) Analyst 143(6):1378-1386; Breibeck & Rompel (2019) Biochimica et Biophysica Acta-General Subjects 1863(2):437-455]. Thus, both rate constant for aggregate formation and hydrophobicity (obtained from Breibeck et al.) followed the order OG >DDM >LDAO (FIG. 4, panel C). The results were in alignment with the HLB value of 13.5 for Triton X-100, indicating lower hydrophobicity and lower tendency to aggregate over time [Slinde & Flatmark (1976) Biochimica et Biophysica Acta-Biomembranes 455(3):796-805].

Kinetics of aggregate formation for Fus1 and Fus2 in different protein concentration and buffer matrix in presence of detergents, OG and DDM was obtained for the conditions in FIG. 2, panels A-C. Given the very high HMW content for OG at 10×CMC for the high concentration DS, all subsequent stability studies were conducted OG at 1×CMC. DDM at 10×CMC was used for all tested conditions. Aggregate formation showed first order kinetics with greatest rate constant for OG followed by DDM. Lower concentration DS at 3 g/L demonstrated higher rate of aggregate formation for Fus1 than Fus2, which can be explained by the structural difference between the two proteins (FIG. 3). For low concentration DS, the rate constants in the range of 10⁻⁴ h⁻¹ were one to two order(s) of magnitude lower in comparison to high concentration DS and protein A eluate of detergent-spiked harvest indicating greater DS stability at lower protein concentrations (FIG. 4, panel D). Unlike the overall HMW formation which was impacted by the extent of initial HMW in the protein matrix, the rate of aggregation showed a strong correlation with the protein and detergent concentration (R²=0.84, ANOVA p value=0.0002) (FIG. 4, panel E).

### 3.2 Detergent and Impurity Clearance

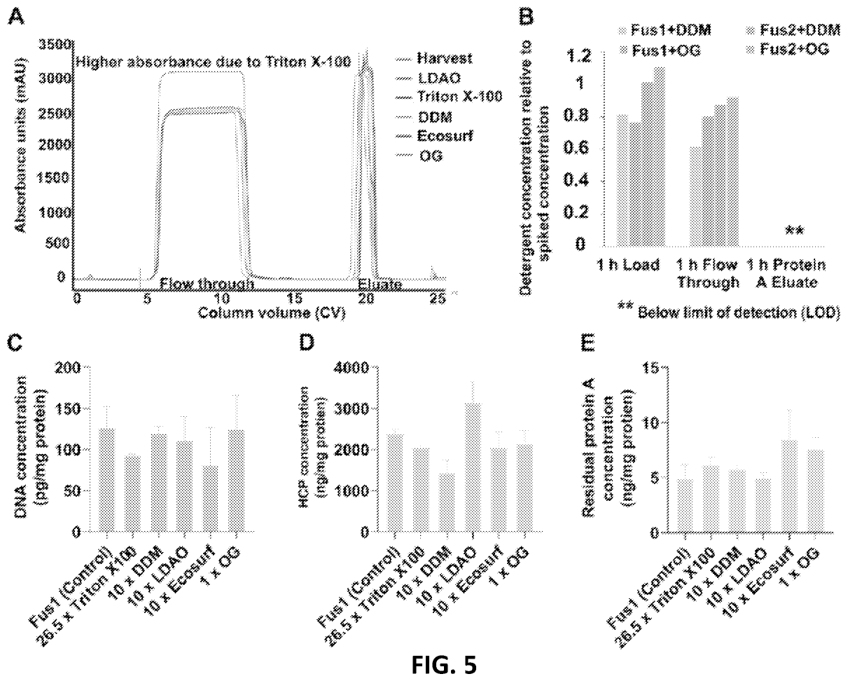

Residual detergent in the final product can impact drug efficacy and immunogenicity. Thus, following the shortlisting of detergents based on VI and stability screening, it was critical to demonstrate detergent clearance through downstream processing. For Fus1 and Fus2, detergent clearance was demonstrated by protein A chromatography following VI of the harvested pool. The protein harvests were spiked with detergents, OG and DDM at 1×CMC (0.68 w/v %) and 10×CMC (0.061 w/v %) respectively. The harvest was then protein A purified, the flow through and eluate were collected and quantified for the respective detergent. Owing to the bind and elute mode of operation, over 75% of the detergent was accounted in the flow through from protein A load. The protein A chromatography demonstrated significant detergent clearance with the eluate detergent concentration being below the limit of detection for both OG (0.01%) and DDM (0.005%). Any unaccounted detergent was likely removed in the column washes preceding protein A elution (FIG. 5, panels A and B). Furthermore, the subsequent polishing chromatography steps provide additional clearance for patient safety.

In addition to detergent, DNA (Deoxyribose nucleic acid), HCP (Host cell protein) and residual protein A were also quantified for protein A eluate of detergent-spiked harvest and the harvest with no detergent as control. All instances of protein A eluate showed comparable DNA, HCP and residual protein A to the control and the Triton X-100-spiked harvest (FIG. 5, panels C-D).

### 3.3 Virus Inactivation

The mechanism of detergent-mediated VI can share similarities with pH-mediated VI and is likely a characteristic of the detergent-protein-virus system under consideration. Detergents could unfold or denature specific viral surface glycoproteins that are critical for the host cell infection as is the case for X-MuLV [Brorson et al. (2003) Biotechnology and Bioengineering 82(3):321-329; Simons & Ehehalt (2002) Journal of Clinical Investigation 110(5):597-603]. Alternately, detergents could inactivate viruses such as HSV-1 through dissolution of the viral lipid envelope or preferential partitioning of the membrane protein into detergent micelles [Welling-Wester et al. (1998) Journal of chromatography A 816(1):29-37]. Three lipid-enveloped viruses commonly used for viral validation studies, X-MuLV, HSV-1 and A-MuLV (Amphotropic Murine Leukemia Virus), were investigated for VI. Any VI condition showing LRV ≥4.0 after a 60 minute hold at 2-8° C., worst case temperature for VI, was considered effective. The extent of inactivation from different detergents was compared with Triton X-100.

### 3.3.1 Detergent

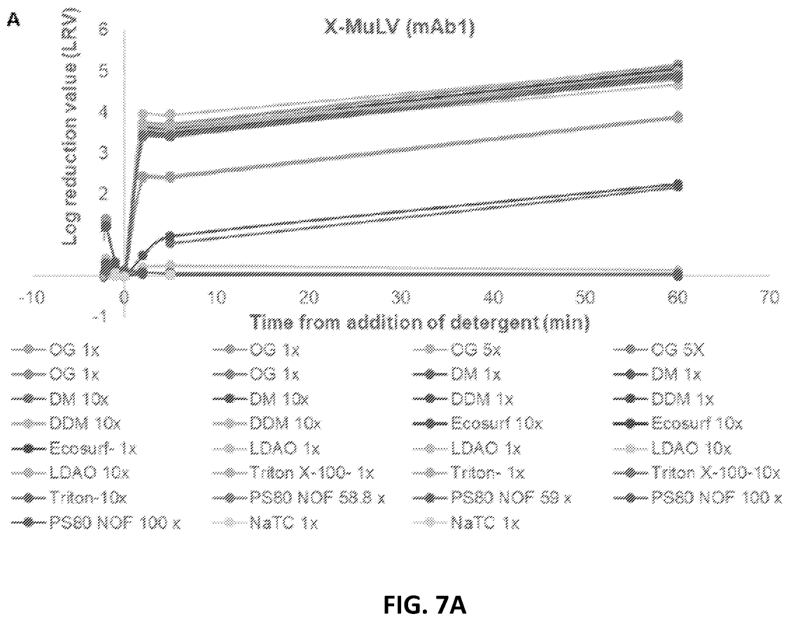

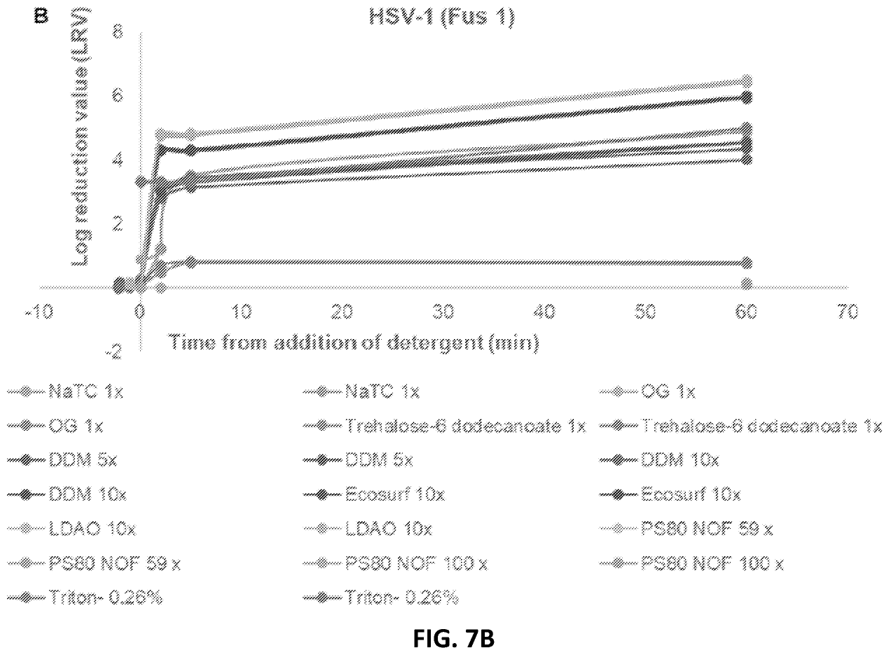

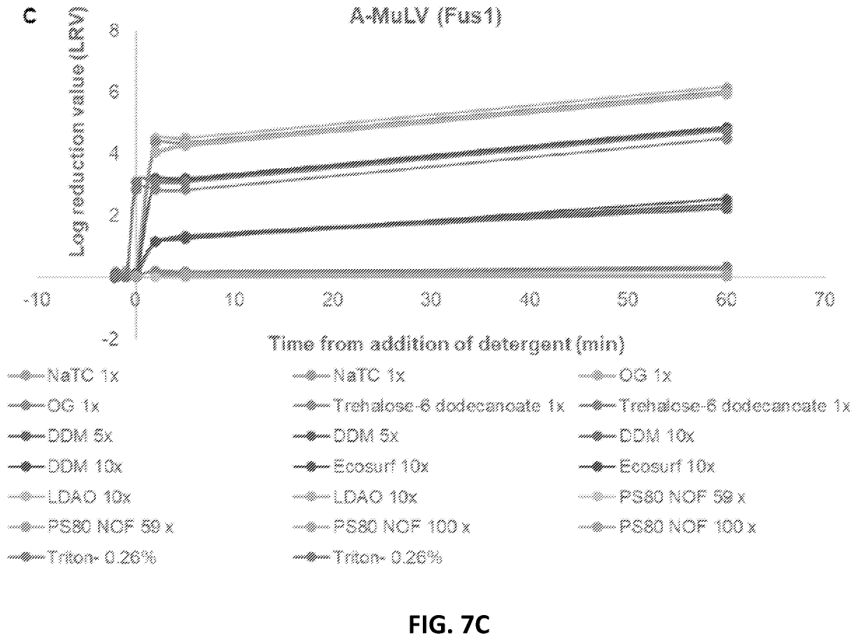

The first therapeutic tested was mAb1, where its harvest was spiked with X-MuLV for a range of detergent concentrations with respect to the CMC (Critical Micelle Concentration) (FIG. 6, panel A). Both conditions of Triton X-100 at 1× and 10×CMC showed LRV ≥4.85±0.5 demonstrating results consistent with a generic bracketed approach to VC [ASTM, E3042-16, Standard Practice for Process Step to Inactivate Rodent Retrovirus with Triton X-100 Treatment. ASTM International, 2016]. Greater than 4.0 LRV was observed for detergents OG, Zwittergent and CG 110 at both the high and low concentrations tested. VI was dependent on concentration for the detergents DM, DDM, LDAO, Ecosurf and CG-650—the latter three showed ≥4.0 LRV at concentrations that are 10×CMC. The efficacy of detergent-induced VI depended on the properties of the detergent such as CMC, hydrophobicity, charge and diffusivity. [Breibeck & Rompel (2019) Biochimica et Biophysica Acta-General Subjects 1863(2):437-455; Feroz et al. (2018) Biophysical Journal 115(2):353-360; Feroz et al. (2016) Biotechnology and Bioengineering 113(10):2122-2130]. Different analogs of the same detergent with varying chain length or headgroups had widely varying molecular properties. The increase in the alkyl chain length by 2 carbon atoms from decyl to dodecyl maltopyranoside, i.e., DM to DDM lead to an almost 10 fold reduction in CMC and increase in hydrophobicity [Feroz et al. (2018) Analyst 143(6):1378-1386; Oliver et al. (2013) PloS One 8(5); Feroz et al. (2019) Biotechnology Progress 35(6):e2859]. Thus, DDM at 10×CMC demonstrated VI of 5.0 LRV for X-MuLV in mAb1 harvest whereas DM at 10×CMC demonstrated only 2.0 LRV of VI as seen in FIG. 6, panel A. Triton X-100 monomers demonstrated faster diffusion across the bilayer than DDM or DM thereby leading to effective bilayer solubilization and VI [Kragh-Hansen et al. (1998) Biophysical Journal 75(6):2932-2946; Lichtenberg et al. (2013) Biophysical Journal 105(2):289-299]. The slower diffusing DM or DDM monomers accumulated in the outer leaflet increasing its curvature until budding or invagination of the membrane to form mixed micelles [Lichtenberg et al. (2013) Biophysical Journal 105(2):289-299]. The slower DM or DDM diffusivity explained the corresponding lower VI in different protein-virus systems. OG showed high inactivation at all conditions due to its greater hydrophobicity as discussed below. With the exception of Zwittergent 3-12 and LDAO, all tested detergents were non-ionic. The zwitterionic nature of these detergents can also explain the greater efficacy of inactivation due to potential electrostatic interaction of the headgroup with the lipid or proteins in the viral envelopes [Conley et al. (2017) Biotechnology and Bioengineering 114(4):813-820]. Despite promising inactivation, some detergents were not carried over for further testing due to lower stability (CG-110), scalability or manufacturing issues (CG-650) and biodegradability or environmental impact (Zwittergent 3-12) [Conley et al. (2017) Biotechnology and Bioengineering 114(4):813-820]. Detailed information about the detergents tested can be found in TABLE 1. The top detergent candidates from the mAb1 inactivation study were carried over for testing with two more lipid-enveloped viruses, HSV-1 and A-MuLV in Fus1 harvest using Triton X-100 at 0.26% (13.5×CMC) as a base case (FIG. 6, panel B, and FIGS. 7A, 7B. and 7C).

### 3.3.2 Protein-Virus Matrix

HSV-1 and A-MuLV was tested in Fus1 and X-MuLV tested in mAb to maintain process consistency while investigating the impact of different proteins and detergents on different viruses. The detergents that showed ≥4.0 LRV for both HSV-1 and A-MuLV in Fus1 include Ecosurf 10×, LDAO 10× and OG 1×CMC (FIG. 6, panel B). DDM at 10×CMC showed at least 4.0 LRV inactivation for X-MuLV in mAb1 and HSV-1 in Fus1 harvest but only 2.2 LRV for A-MuLV in Fus1 after 60 minutes at 2-8° C. This lower extent of inactivation for DDM can be attributed to the protein or virus being tested in addition to the detergent properties Aside from morphological differences between A-MuLV and X-MuLV which are homologous in nature, the difference in VI resistance of these two viruses could also be attributed to the difference in the harvest cell culture composition and modalities of the two proteins tested, A-MuLV being tested in fusion protein, Fus1 and X-MuLV being tested in mAb.

Similar observation was made by Conley et al. who saw lower LRV of ˜3.0 for fusion protein at 2-8° C. compared to LRV of 4.0 for mAb under the same conditions of LDAO-mediated inactivation. Their study showed that LDAO inactivation improved to an LRV of ˜4.0 for the fusion protein at room temperature compared to ˜3.0 at 2-8° C. indicating faster kinetics at higher temperature. Conley et al. also demonstrated the time dependence of inactivation with greater inactivation being observed for longer incubation as was confirmed by our data (FIG. 7, panels A-C) [Conley et al. (2017) Biotechnology and Bioengineering 114(4):813-820].

For all detergent-mediated VI conditions tested with the exception of DDM 10×CMC, HSV-1 showed greater inactivation than either A-MuLV or X-MuLV (FIG. 6). No inactivation was observed for any of the viruses in presence of Polysorbate 80 (PS80) which is commonly employed for solvent-detergent inactivation of plasma-derived products [Espindola et al. (2006) Journal of Virological Methods 134(1-2):171-175]. Only HSV-1 in Fus1 showed inactivation greater than 4.0 LRV for sodium taurocholate (NaTC) at 1×CMC. Although DDM showed greater than 4.0 LRV at both 5× and 10×CMC for HSV-1, it only showed 2.2 LRV for A-MuLV. The larger size of HSV-1 (120-200 nm) compared to MuLV (80-120 nm) could lead to easier translocation of detergent across the lipid bilayer at a lower energy penalty leading to greater VI [Hu et al. (2013) Journal of Physical Chemistry B 117(39):11641-11653; Meingast & Heldt (2020) Biotechnology Progress 36(2):e2931]. The lower inactivation of MuLV can also be attributed to the greater resistance to detergent-mediated solubilization of the lipid envelope due to higher fraction of cholesterol or sphingomyelin content giving rise to detergent-resistant microdomains (DRMs) compared to HSV-1 [Simons & Ehehalt (2002) Journal of Clinical Investigation 110(5):597-603; van Genderen et al. (1994) Virology 200(2):831-836; Chan et al. (2008) Journal of virology 82(22): 11228-11238]. Cholesterol intercalates in the acyl chains of sphingolipids thus reducing membrane fluidity and giving rise to DRMs. Membrane proteins critical for viral infection are often associated with DRMs. As such, DRM-resistance to solubilization can significantly reduce the extent of detergent-mediated inactivation [Simons & Ehehalt (2002) Journal of Clinical Investigation 110(5):597-603; Beer et al. (2005) Virology Journal 2(1):36]. MuLV have a significantly higher sphingomyelin content compared to HSV-1 (22.5% versus 3.1%) and a lower phosphatidylcholine content by comparison (19% versus 51.2%). The lower inactivation of MuLV observed can thus be attributed to the relatively higher cholesterol and sphingomyelin content [van Genderen et al. (1994) Virology 200(2):831-836; Chan et al. (2008) Journal of virology 82(22): 11228-11238]. Garner et al. showed that while Triton X-100 only selectively solubilizes non-DRM regions, OG dissolved bilayers completely which may explain the greater extent of inactivation by OG [Garner et al (2008) Biophysical Journal 94(4):1326-1340].

### 4. Supplemental Information

### 4.1 Aggregation Kinetics

Protein unfolding was modeled by the Lumry-Eyring framework, beginning with first order reversible protein unfolding (RLS=rate limiting step) followed by higher order aggregation [Shukla et al. (2007) Journal of Chromatography A 1171(1-2):22-28; Kendrick et al. (1998) Proceedings of the National Academy of Sciences USA 95(24):14142-14146]. Given N is the monomer species, A is the transient unfolded state of monomer and Aₘ is the aggregate species formed, the steps in aggregation can be represented by equation (1) and (2)

$$\begin{matrix}{k}_{1} \\ N ⇋ A \\ {k}_{- 1}\end{matrix} \qquad ( 1 )$$

$${A}_{m} + A {⟶}_{{k}_{m}} {A}_{m + 1} \qquad ( 2 )$$

The kinetics of aggregation can be represented by equation (3) where time is represented by t

$$\frac{dN}{dt} = - {k}_{1} [ N ] + {k}_{- 1} [ A ] \qquad ( 3 )$$

Assuming A is rapidly converted to aggregates, [A]=0, Nₒ is the initial monomer % at time, t=0, aggregation follows first order kinetics with k₁ being the rate constant of aggregation as shown in Equation (5)

$$\frac{dN}{dt} = - {k}_{1} [ N ] \qquad ( 4 )$$

Aggregation can thus be modeled by first order kinetics as seen in equation (5)

$$ln \frac{[ N ]}{[ {N}_{o ]}} = - {k}_{1} t \qquad ( 5 )$$

### 5. Conclusion

A detailed strategy to screen conditions for VI is presented. This strategy started with assessing the therapeutic stability in presence of detergent, followed by evaluating detergent and impurity clearance in subsequent downstream steps. Using this approach, we screened different non-ionic and zwitterionic detergents and low pH sensitive fusion proteins, Fus1 and Fus2. We used statistical analysis to determine the factors driving protein instability when subjected to detergent-mediated VI. Aggregation was directly linked to extrinsic process conditions of hold times, as well as detergent and protein concentrations. High concentrations of both protein and detergent were worst case for HMW formation demonstrating first order aggregation kinetics. Aggregation was also impacted by intrinsic factors including initial aggregate levels, amino acid sequence and detergent properties. The greatest rate of aggregation was observed for the detergents in the same sequence as their relative hydrophobicity-OG, DDM followed by LDAO. Fus1 had a slightly higher tendency to aggregate than Fus 2 which was attributed to the greater hydrophobicity of Fus1 as demonstrated by SAP modeling.

Testing the efficacy of the inactivating agent with BSL2 viruses at specially trained third party testing sites was the final step in the screening process. Withholding VC studies until after therapeutic characterization offered the advantage of eliminating conditions that did not meet the stability criteria thereby leading to savings in cost and time. The sugar-based biodegradable detergent, DDM showed robust inactivation of LRV ≥4.0 for X-MuLV (mAb1) and HSV-1 (Fus1) in addition to other formerly characterized detergents, Triton X-100, OG, LDAO and Ecosurf. The extent of inactivation for different virus-protein systems depended on extrinsic conditions such as the duration of incubation and concentration of the detergent tested.

### Example 2

### Viral Inactivation Evaluation

Viral inactivation studies were conducted to evaluate how effectively each detergent candidate inactivates model viruses. Two model viruses, i.e., amphotrophic murine leukemia virus (A-MuLV), and herpes simplex virus type 1 (HSV-1), were used. For each detergent, solutions of different concentrations were prepared. The detergent solutions were then mixed with harvest materials of abatacept spiked with the two model viruses. The harvest materials were then incubated with detergent solution for 60 minutes. The viral activity was tested after 0, 15, 30, and 60 minutes after incubation. Such time points were chosen to comply with regulatory guidance provided by FDA. Per ICH Q5A (Viral Safety Evaluation of Biotechnology Products Derived from Cell Lines of Human or Animal Origin), a minimum of 3 time points are needed to determine viral inactivation kinetics, and at least 1 time point less than the minimum inactivation time. The filing would claim the LRV from the 60-minute time point.

The detergent concentration used for viral inactivation are presented below in TABLE 2:

TABLE 2
Detergents tested for viral inactivation

| | | | Final |
|---|---|---|---|
| | CMC | | Concentration |
| | Values | | in Harvest |
| | in % | | Material % |
| Detergent | (g/100 mL) | X CMC^{a} | (g/100 mL) |
| n-Octyl-β-D- | 0.6800 | 0.5 | 0.34 |
| Glucopyranoside (OG) | | | |
| n-Octyl-β-D- | 0.6800 (OG) | 0.5 (OG) | 0.34 (OG) |
| Glucopyranoside (OG) | 0.0061 (DDM) | 5 (DDM) | 0.0305 (DDM) |
| n-Dodecyl-β-D- | | | |
| Maltopyranoside (DDM) | | | |
| Combination | | | |
| EcoSurf ™EH9 | 0.1066 | 5 | 0.533 |
| Lauryldimethylamine | 0.0032 | 5 | 0.016 |
| Oxide (LDAO) | | | |

^{a}CMC indicates critical micelle concentration. It is defined as the minimum concentration of a surfactant to form micelle, and usually used as a concentration unit for surfactants.

### Protein A Chromatography Evaluation

Protein A chromatography is a typical unit operation step after detergent-based viral inactivation. The use of different detergents might have an impact on the process parameters and quality attributes of the protein A load and pool materials, as well as the final drug substances. Hence, the harvest materials spiked with detergents was passed through a protein A chromatography. The pool quality attributes, including high molecular weight (HMW) species, sialic acid content, protein deamidation and oxidation profile, and impurity profile, of the eluate out of the protein A chromatography were tested to evaluate the impact of different detergents.

The detergent concentrations used for protein A chromatography evaluation are presented in TABLE 3.

TABLE 3
Detergent used for protein A chromatography evaluation

| | | | Final |
|---|---|---|---|
| | CMC | | Concentration |
| | Values | | in Harvest |
| | in % | | Material % |
| Detergent | (g/100 mL) | X CMC | (g/100 mL) |
| n-Octyl-β-D- | 0.6800 (OG) | 0.5 (OG) | 0.34 (OG) |
| Glucopyranoside (OG) | 0.0061 (DDM) | 5 (DDM) | 0.0305 (DDM) |
| n-Dodecyl-β-D- | 0.6800 (OG) | 0.5 (OG) | 0.34 (OG) |
| Maltopyranoside (DDM) | 0.0061 (DDM) | 7.5 (DDM) | 0.0458 (DDM) |
| Combination | 0.6800 (OG) | 0.5 (OG) | 0.34 (OG) |
| | 0.0061 (DDM) | 10 (DDM) | 0.0610 (DDM) |
| EcoSurf ™EH9 | 0.1066 | 5 | 0.533 |
| | 0.1066 | 7.5 | 0.7995 |
| | 0.1066 | 10 | 1.066 |
| Lauryldimethylamine | 0.0032 | 10 | 0.032 |
| Oxide (LDAO) | | | |

The performance and effectiveness of different detergents was evaluated from all aspects that are relevant to the quality of final drug substances. These aspects included the effectiveness in viral inactivation, the effects on protein aggregation, sialic acid content, protein stability, and impurity clearance. The detergent concentration was evaluated using the “CMC” unit. For example, Triton X-100 26.9× indicates the detergent was Triton X-100 and the concentration was 26.9 times of its critical micelle concentration, and OG 0.5×DDM 5× indicates the detergent combination includes OG with 0.5 times of its critical micelle concentration and DDM with 5 times of its critical micelle concentration.

### Viral Inactivation Effectiveness

Viral inactivation/clearance is usually measured in units of Log Reduction Value (LRV) where it is defined as:

$$LRV = - {log}_{10} \frac{{[ Virus in pfu ]}_{P r o d u c t p o o l}}{{[ Virus in pfu ]}_{L o a d / F e e d}}$$

For the manufacturing of biologics, regulatory agencies such as FDA usually require demonstration of a total of at least 6 LRV with at least 2 orthogonal techniques. For a single viral inactivation step, at least 4 LRV is usually required. The viral inactivation effect depends on several factors including incubation temperature, time, and detergent concentration. Higher temperature, longer incubation duration, and higher detergent concentration typically benefits viral inactivation. For abatacept process J, the viral inactivation is conducted at room temperature for 60 minutes, and the model viruses used for viral inactivation studies are A-MuLV and HSV-1.

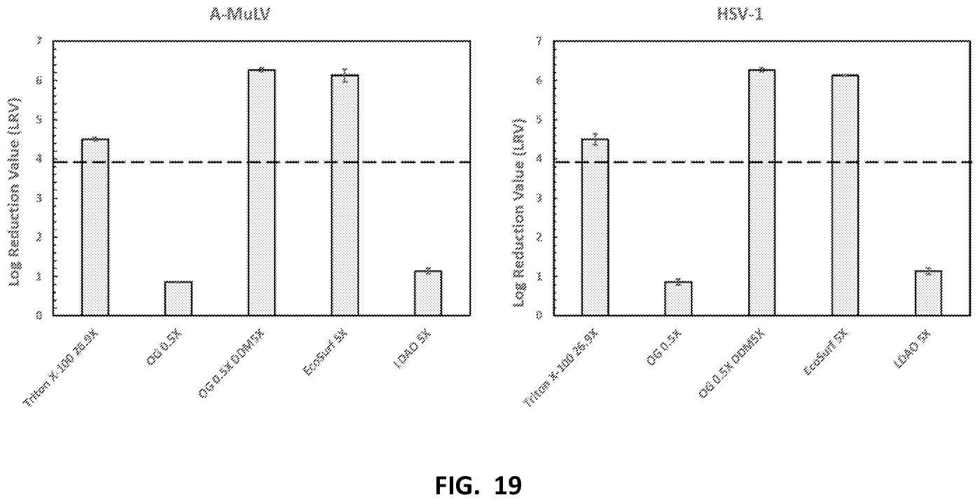

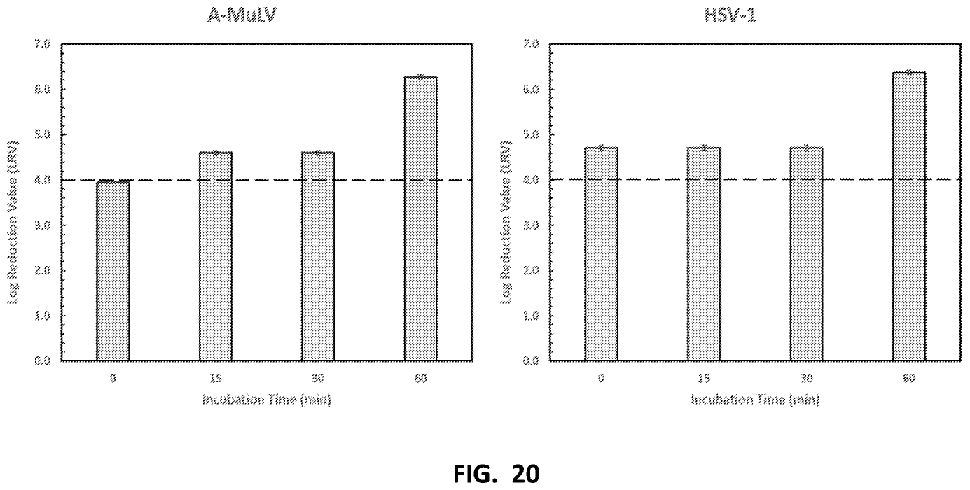

The viral inactivation effectiveness results of different detergents with A-MuLV and HSV-1 were presented in FIG. 19. Results indicated that Triton X-100 26.9×, OG 0.5×DDM 5×, and EcoSurf 5× all showed satisfactory viral inactivation (LRV ≥4) for both model viruses after 60 minutes of incubation. Additionally, the performance of OG 0.5×DDM5× was even better than Triton X-100, which is the current detergent in-use for abatacept process J. The viral inactivation effectiveness results of the detergent combination, i.e., OG 0.5×DDM 5×, at different incubation time points are presented in FIG. 20. Results indicated that the detergent combination, i.e., OG 0.5×DDM 5×, showed satisfactory viral inactivation (LRV ≥4) for both model viruses right after detergent addition, i.e., incubation time at 0 minute. Besides, when incubation time was 15, 30, and 60 minutes, all the detergent combination showed satisfactory viral inactivation. Hence, OG 0.5×DDM 5× was determined to be a qualified substitute of Triton X-100 in terms of its viral inactivation effectiveness. Given that the viral inactivation effectiveness of a detergent also positively correlates with its concentration, we concluded that any detergent combination candidate with higher concentration than the one tested here (OG 0.5×DDM 5×) would also have satisfactory viral inactivation effects.

### Effects on Protein Aggregation

The chemical structure of different detergents can have an impact on the formation of protein aggregation, which is usually characterized by the percentage of high molecular weight species (HMW). The HMW level is in the release specifications of abatacept drug substance (DS), hence an important parameter to monitor. The HMW level in the Protein A pool chromatography is directly related to DS HMW level. According to several studies, for abatacept process J, the HMW species are cleared mostly via the hydrophilic interaction chromatography (HIC) step after Protein A chromatography. The HIC step has a clearance capacity of around 30%. Hence, if the addition of a detergent leads to over 30% HMW in the protein pool, that may create high risk to the final DS quality.

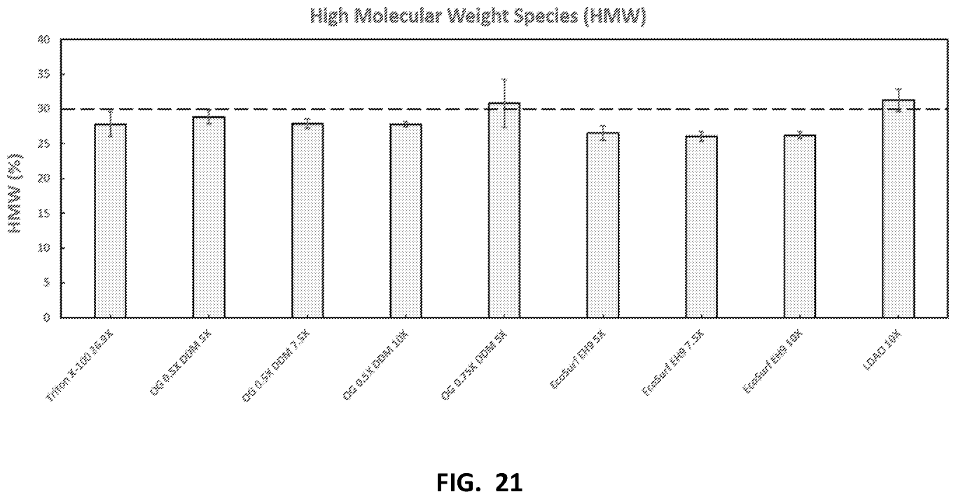

The effects of different detergents on protein aggregation in the Protein A pool are presented in FIG. 21. The OG/DDM detergent combination candidates, i.e., OG 0.5×DDM 5×, OG 0.5×DDM 7.5×, OG 0.5×DDM10×, and OG 0.75×DDM 5×, were compared with the current detergent in-use, i.e., Triton X-100, and two other detergents (ECOSURF™ and LDAO). Results indicated that three detergent OG/DDM combination candidates, i.e., OG 0.5×DDM 5×, OG 0.5×DDM 7.5×, and OG 0.5×DDM 10×, all led to a HMW level below 30%. Additionally, the HMW level associated with those three detergents was similar to that with Triton X-100, the current detergent in use, and ECOSURF™ EH9 and LDAO. Hence, it was concluded that the high molecular weight species (HMW) caused by the detergent combination candidate could be safely cleared by the current process capabilities. The OG/DDM detergent combination would not cause severe protein aggregation that might affect the quality of final drug substance, and could substitute for Triton X-100 from this perspective.

### Sialic Acid Content

Glycosylation of proteins is another critical phenomenon that could affect the protein potency and aggregation. Sialic acids refer to neuraminic acid modified by the addition of an acetyl group (N-acetylneuraminic acid/NANA) or a glycol group (N-glycolylneuraminic acid/NGNA). Both NANA and NGNA were set to be critical product quality attributes. For abatacept process J, the acceptable range was set to be 8 to 12 (moles/mole protein) for NANA, and lower than 1.3 (moles/mole protein) for NGNA.

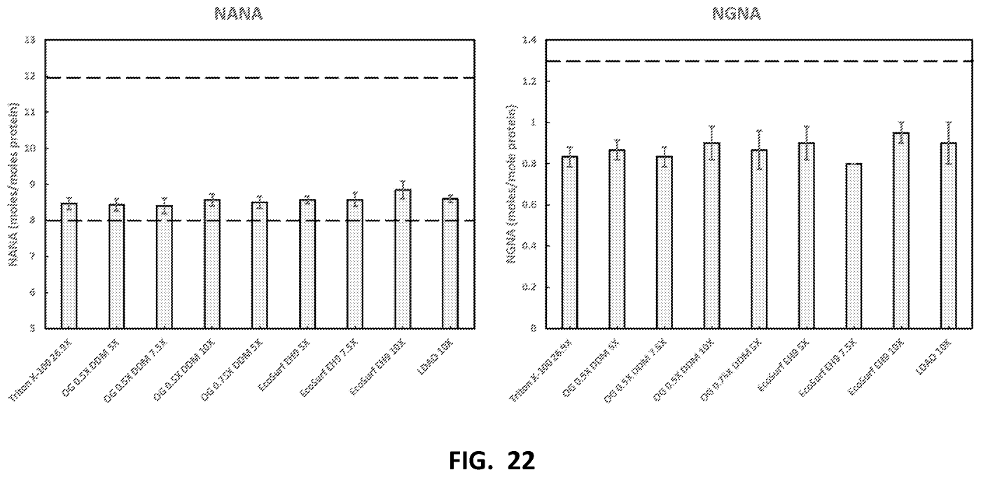

FIG. 22 presents the effects of different detergents on the NANA and NGNA level of ProA pool processed with different detergents. Results indicated that all detergents showed satisfactory NANA and NGNA outcomes. No values were out of the acceptable range and no major risk was observed. Hence, it was concluded that the OG/DDM detergent combinations, i.e., OG 0.5×DDM 5×, OG 0.5×DDM 7.5×, OG 0.5×DDM10×, could be satisfactory substitutes for Triton X-100 because they lead to satisfactory sialic acid outcome.

### Protein Stability

Methionine oxidation and asparagine/glutamine deamidation of post-translationally modified recombinant proteins could be a major concern for DS attributes, particularly immunogenicity and potency. For abatacept DS, the acceptable upper limit for oxidation is 1.3%, and the acceptable range for deamidation was 5.9%. The ProA pool deamidation and oxidation profile was closely related with the DS profile. Hence the same set of acceptable range applied to ProA pool.

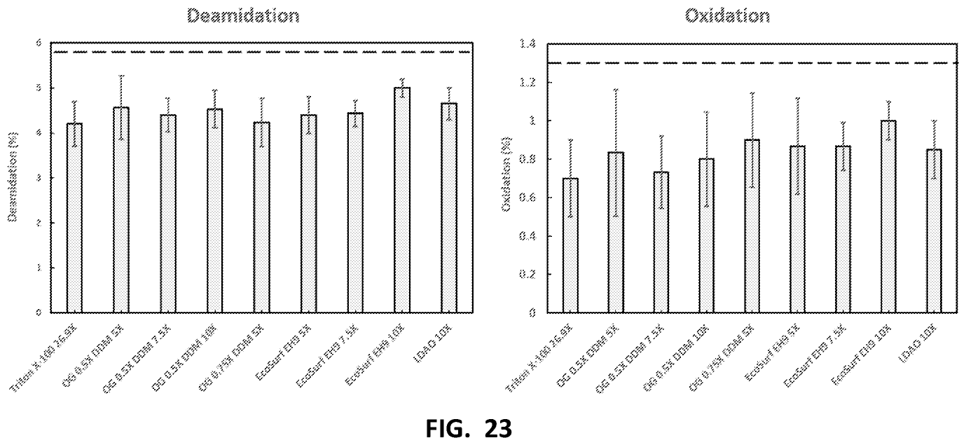

FIG. 23 presents the effects of different detergents on protein A pool deamidation and oxidation profile. Results indicated that the deamidation and oxidation values associated with all detergents were within the acceptable range. No major risk was observed with any specific detergent candidate. Hence, it is concluded that the OG/DDM detergent combinations, i.e., OG 0.5×DDM 5×, OG 0.5×DDM 7.5×, OG 0.5×DDM10×, could be satisfactory substitutes for Triton X-100 because they would not lead to out-of-range oxidation and deamidation profiles.

### Impurity Clearance

Several impurities, i.e., host cell protein (HCP), residual protein A ligand (rProA), and DNA, could exist in the protein A pool. These impurities need to be controlled under a certain level such that they could not create potential safety issues. Release specifications of abatacept process J set the acceptable range for HCP and DNA to be ≤10 ng/mg and 1.0 μg/mg in drug substance, respectively. Although there was no acceptable range set for protein A pool, previous studies indicated that the maximum HCP and DNA in protein A pool should be 56000 ppb and 5000 ppm, respectively. No specification was set for rProA, but previous studies indicated that the maximum rProA in protein A pool was typically around 2.38 μg/mL.

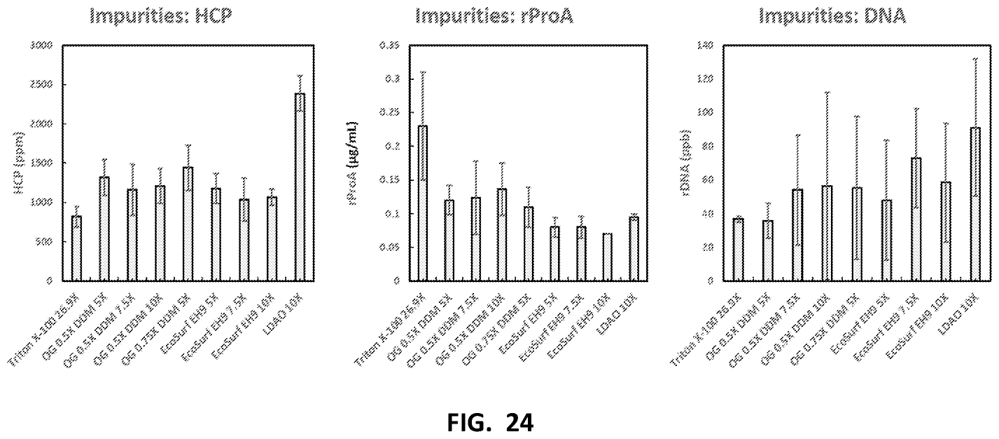

Results indicated that the impurity profile results for all OG/DDM detergent combination candidates were below the acceptable range. FIG. 24. Hence, it was concluded that the OG/DDM detergent combination candidates, OG 0.5×DDM 5×, OG 0.5×DDM 7.5×, OG 0.5×DDM 10×, could be substitutes for Triton X-100 because they lead to satisfactory impurity profiles.

### CONCLUSION

Three OG/DDM detergent combination candidates, i.e., OG 0.5×DDM 5×, OG 0.5×DDM 7.5×, OG 0.5×DDM 10×, were identified as suitable, environmentally sustainable substitutes of Triton X-100, for the viral inactivation of abatacept process J. The viral inactivation effectiveness, effects on protein aggregation, sialic acid content, protein stability and impurity profile of the detergent combination candidates were evaluated and compared with Triton X-100, LDAO and ECOSURF™ EH9. Results indicated that first, the OG/DDM detergent combination candidates showed comparable viral inactivation effects as compared to Triton X-100. Additionally, no severe risk was observed for the OG/DDM detergent combination candidates regarding their effects on protein aggregation, sialic acid content, stability, and impurity profile. Each single detergent in the combination is also recognized by the European Medicines Agency (EMA) as environmentally sustainable. Hence, the detergent combination candidates are satisfactory environmentally sustainable substitutes of Triton X-100 for the viral inactivation of abatacept process J.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described herein.

All publications, patent applications, patents, and other references mentioned herein are incorporated by reference in their entirety. Database entries and electronic publications disclosed in the present disclosure are incorporated by reference in their entireties. The version of the database entry or electronic publication incorporated by reference in the present application is the most recent version of the database entry or electronic publication that was publicly available at the time the present application was filed. The database entries corresponding to gene or protein identifiers (e.g., genes or proteins identified by an accession number or database identifier of a public database such as Genbank, Refseq, or Uniprot) disclosed in the present application are incorporated by reference in their entireties. The gene or protein-related incorporated information is not limited to the sequence data contained in the database entry. The information incorporated by reference includes the entire contents of the database entry in the most recent version of the database that was publicly available at the time the present application was filed. In case of conflict, the present specification, including definitions, will control. In addition, the materials, methods, and examples are illustrative only and not intended to be limiting.

What is claimed is:

1. A method of inactivating a virus in a product feedstream in a manufacturing process of a therapeutic protein, comprising contacting the product feedstream with n-Dodecyl-β-D-Maltopyranoside (DDM).

2. The method of claim 1, further comprising contacting the product feedstream with n-Octyl-β-D-Glucopyranoside (OG).

3. A method of inactivating a virus in a product feedstream in a manufacturing process of a therapeutic protein, comprising contacting the feed stream with a composition comprising DDM and OG.

4. The method of any one of claims 1 to 3, wherein the DDM is present at a concentration which is at least about 1, at least about 2, at least about 3, at least about 4, at least about 5, at least about 6, at least about 7, at least about 7.5, at least about 8, at least about 9, at least about 10, at least about 11, at least about 12, at least about 13, at least about 14, at least about 15, at least about 16, at least about 17, at least about 18, at least about 19, or at least about 20 times its critical micelle concentration (CMC).

5. The method of any one of claims 1 to 4, wherein the DDM is present at a concentration which is between about 1 to about 20 times its CMC, between about 1 to about 19 times its CMC, between about 1 to about 18 times its CMC, between about 1 to about 17 times its CMC, between about 1 to about 16 times its CMC, between about 1 to about 15 times its CMC, between about 1 to about 14 times its CMC, between about 1 to about 13 times its CMC, between about 1 to about 12 times its CMC, between about 1 to about 11 times its CMC, between about 1 to about 10 times its CMC, between about 1 to about 9 times its CMC, between about 1 to about 8 times its CMC, between about 1 to about 7 times its CMC, between about 1 to about 6 times its CMC, between about 1 to about 5 times its CMC, between about 1 to about 4 times its CMC, between about 1 to about 3 times its CMC, or between about 1 to about 2 times its CMC.

6. The method of any one of claims 1 to 5, wherein the DDM is present at a concentration between about 5 to about 10 times its CMC.

7. The method of any one of claims 2 to 6, wherein the OG is present at a concentration which is at least about 0.1 times its CMC, at least about 0.2 times its CMC, at least about 0.3 times its CMC, at least about 0.4 times its CMC, at least about 0.5 times its CMC, at least about 0.6 times its CMC, at least about 0.7 times its CMC, at least about 0.8 times its CMC, at least about 0.9 times its CMC, or at least about 1 time its CMC.

8. The method of any one of claims 2 to 7, wherein the OG is present at a concentration between about 0.1 to about 1 times its CMC.

9. The method of any one of claims 2 to 8, wherein the OG is present at a concentration which is about 0.5 times its CMC, and DDM is present at a concentration which is between about 5 to about 10 times its CMC.

10. The method of any one of claims 2 to 9, wherein
(i) the OG is present at a concentration which is about 0.5 times its CMC, and the DDM is present at a concentration which is about 5 times its CMC,
(ii) the OG is present at a concentration which is about 0.5 times its CMC, and the DDM is present at a concentration which is about 7.5 times its CMC,
(iii) the OG is present at a concentration which is about 0.5 times its CMC, and the DDM is present at a concentration which is about 10 times its CMC, or
(iv) the OG is present at a concentration which is about 0.75 times its CMC, and the DDM is present at a concentration which is about 5 times its CMC.

11. The method of any one of claims 1 to 10, wherein the product feedstream comprises a harvest from a bioreactor, a chromatography load, a chromatography eluate, a filtration load, a filtrate, or any combination thereof.

12. The method of claim 11, wherein the chromatography eluate is a Protein A chromatography eluate.

13. The method of any one of claims 1 to 12, wherein the virus comprises a lipid-enveloped virus.

14. The method of claim 13, wherein the lipid-enveloped virus is a retrovirus.

15. The method of claim 14, wherein the retrovirus is A-MuLV.

16. The method of claim 13, wherein the lipid-enveloped virus is a herpesvirus.

17. The method of claim 16, wherein the herpesvirus is HSV-1.

18. The method of any one of claims 1 to 17, wherein inactivating the lipid-enveloped virus comprises a log reduction value (LRV) of at least about 4, at least about 5, at least about 6, at least about 7, at least about 8, at least about 9, or at least about 10, and wherein the LRV is calculated as:
$$LRV = - {log}_{10} \frac{{[ Virus in pfu ]}_{P r o d u c t p o o l}}{{[ Virus in pfu ]}_{L o a d / F e e d}}$$

19. The method of claim 18, wherein the LRV is at least about 4.

20. The method of any one of claims 1 to 19, wherein the contacting occurs for at least about 15 minutes, at least about 30 minutes, at least about 60 minutes, at least about 70 minutes, at least about 80 minutes, at least about 90 minutes, at least about 100 minutes, at least about 110 minutes, or at least about 120 minutes.

21. The method of any one of claims 1 to 20, wherein the product feedstream contains an amount of high molecular weight (HMW) species of the therapeutic protein after the contacting below about 30%, below about 29%, below about 28%, below about 27%, below about 26%, below about 25%, below about 24%, below about 23%, below about 22%, below about 21%, below about 20%, below about 19%, below about 18%, below about 17%, below about 16%, below about 15%, below about 14%, below about 13%, below about 12%, below about 11%, below about 10%, below about 9%, below about 8%, below about 7%, below about 6%, or below about 5% of the total amount of therapeutic protein.

22. The method of any one of claims 1 to 21, wherein the therapeutic protein has an amount of glycosylation after the contacting which is the same or changed (increased or decreased) by about 10%, about 9%, about 8%, about 7%, about 6%, about 5%, about 4%, about 3%, about 2%, or about 1% compared to the amount of glycosylation of the therapeutic protein prior to the contacting.

23. The method of any one of claims 1 to 22, wherein the therapeutic protein has an amount of N-acetylneuraminic acid (NANA) between about 8 to about 12 moles/mole therapeutic protein, and/or an amount of N-glycolylneuraminic acid (NGNA) less than or equal to about 1.3 moles/mole therapeutic protein after the contacting.

24. The method of any one of claims 1 to 23, wherein the therapeutic protein has an amount of deamidation of after the contacting of less about 5.9% of the total amount of therapeutic protein.

25. The method of any one of claims 1 to 24, wherein the therapeutic protein has an amount of oxidation after the contacting after the contacting of less about 1.3% of the total amount of therapeutic protein.

26. The method of any one of claims 1 to 25, wherein the product feedstream has a residual amount of host cell proteins after the contacting at a concentration of less than about 5,000 ppm, less than about 4,000 ppm, less than about 3,000 ppm, less than about 2,000 ppm, less than about 1,500 ppm, less than about 1,000 ppm, less than about 900 ppm, less than about 800 ppm, less than about 700 ppm, less than about 600 ppm, or less than about 500 ppm.

27. The method of claim 26, wherein the residual amount of host cell proteins in the product feedstream after the contacting is at a concentration between about 500 ppm and about 2,000 ppm.

28. The method of any one of claims 1 to 27, wherein the product feedstream has a residual amount of DNA after the contacting at a concentration of less than about 80,000 ppb, less than about 75,000 ppb, less than about 70,000 ppb, less than about 65,000 ppb, less than about 60,000 ppb, less than about 59,000 ppb, less than about 58,000 ppb, less than about 57,000 ppb, or less than about 56,000 ppb.

29. The method of any one of claims 1 to 21, wherein the product feedstream has a residual amount of DNA after the contacting at a concentration of less than about 500 ppb, less than about 450 ppb, less than about 400 ppb, less than about 350 ppb, less than about 300 ppb, less than about 250 ppb, or less than about 200 ppb.

30. The method of claim 29, wherein the residual amount of DNA in the product feedstream after the contacting is between about 50 and about 200 ppb.

31. The method of any one of claims 1 to 30, wherein the product feedstream has a residual amount of Protein A after the contacting of less than about 1.0 μg/mL, about 0.9 μg/mL, about 0.8 μg/mL, about 0.7 μg/mL, about 0.6 μg/mL, about 0.5 μg/mL, about 0.4 μg/mL, about 0.3 μg/mL, or about 0.2 μg/mL.

32. The method of any one of claims 1 to 31, wherein the therapeutic protein comprises an antibody, antibody fragment, a fusion protein, a naturally occurring protein, a chimeric protein, or any combination thereof.

33. The method of any one of claims 1 to 32, wherein the therapeutic protein comprises a CTLA4 domain.

34. The method of any one of claims 1 to 33, wherein the therapeutic protein is a fusion protein.

35. The method of claim 34, wherein the fusion protein comprises an Fc portion.

36. The method of any one of claims 1 to 35, wherein the therapeutic protein is abatacept or belatacept.

37. The method of claim 37, wherein the therapeutic protein is an abatacept composition comprising an amino acid sequence as set forth in SEQ ID NO:3, a fragment thereof, or a combination thereof.

38. The method of claim 36, wherein the therapeutic protein is a belatacept composition comprising an amino acid sequence as set forth in SEQ ID NO.4, a fragment thereof, or a combination thereof.

39. A composition for inactivating a virus in a product feedstream in a manufacturing process of a therapeutic protein, wherein the composition comprises n-Dodecyl-β-D-Maltopyranoside (DDM).

40. The composition of claim 40, wherein the composition further comprises n-Octyl-β-D-Glucopyranoside (OG).

41. A composition of inactivating a virus in a product feedstream in a manufacturing process of a therapeutic protein, wherein the composition comprises DDM and OG.

42. The composition of any one of claims 39 to 41, wherein the DDM is present at a concentration which is at least about 1, at least about 2, at least about 3, at least about 4, at least about 5, at least about 6, at least about 7, at least about 7.5, at least about 8, at least about 9, at least about 10, at least about 11, at least about 12, at least about 13, at least about 14, at least about 15, at least about 16, at least about 17, at least about 18, at least about 19, or at least about 20 times its critical micelle concentration (CMC).

43. The composition of any one of claims 39 to 42, wherein the DDM is present at a concentration which is between about 1 to about 20 times its CMC, between about 1 to about 19 times its CMC, between about 1 to about 18 times its CMC, between about 1 to about 17 times its CMC, between about 1 to about 16 times its CMC, between about 1 to about 15 times its CMC, between about 1 to about 14 times its CMC, between about 1 to about 13 times its CMC, between about 1 to about 12 times its CMC, between about 1 to about 11 times its CMC, between about 1 to about 10 times its CMC, between about 1 to about 9 times its CMC, between about 1 to about 8 times its CMC, between about 1 to about 7 times its CMC, between about 1 to about 6 times its CMC, between about 1 to about 5 times its CMC, between about 1 to about 4 times its CMC, between about 1 to about 3 times its CMC, or between about 1 to about 2 times its CMC.

44. The composition of any one of claims 39 to 43, wherein the DDM is present at a concentration between about 5 to about 10 times its CMC.

45. The composition of any one of claims 40 to 44, wherein the OG is present at a concentration which is at least about 0.1 times its CMC, at least about 0.2 times its CMC, at least about 0.3 times its CMC, at least about 0.4 times its CMC, at least about 0.5 times its CMC, at least about 0.6 times its CMC, at least about 0.7 times its CMC, at least about 0.8 times its CMC, at least about 0.9 times its CMC, or at least about 1 time its CMC.

46. The composition of any one of claims 40 to 45, wherein the OG is present at a concentration between about 0.1 to about 1 times its CMC.

47. The composition of any one of claims 40 to 46, wherein the OG is present at a concentration which is about 0.5 times its CMC, and DDM is present at a concentration which is between about 5 to about 10 times its CMC.

48. The composition of any one of claims 40 to 47, wherein
(i) the OG is present at a concentration which is about 0.5 times its CMC, and the DDM is present at a concentration which is about 5 times its CMC,
(ii) the OG is present at a concentration which is about 0.5 times its CMC, and the DDM is present at a concentration which is about 7.5 times its CMC,
(iii) the OG is present at a concentration which is about 0.5 times its CMC, and the DDM is present at a concentration which is about 10 times its CMC, or
(iv) the OG is present at a concentration which is about 0.75 times its CMC, and the DDM is present at a concentration which is about 5 times its CMC.

49. A method to treat a disease or condition comprising administering to a subject a therapeutic protein manufactured by a process comprising a viral inactivation step according to the method of any one of claims 1 to 39, or a viral inactivation step comprising the use of the composition of any one of claims 40 to 48.

50. A pharmaceutical composition manufactured by a process comprising a viral inactivation step according to the method of any one of claims 1 to 39, or a viral inactivation step comprising the use of the composition of any one of claims 40 to 48.

51. A method of manufacture a therapeutic protein comprising a viral inactivation step according to the method of any one of claims 1 to 39, or a viral inactivation step comprising the use of the composition of any one of claims 40 to 48.

52. A kit comprising a composition according to any one claims 40 to 48, and instructions for inactivating a virus.

53. The kit according to claim 52, wherein the instructions for inactivating are according to the method of any one of claim 40 to 48.


SEQUENCE LISTING (4 sequences)

SEQ ID NO: 1 - protein, 383 aa, synthetic construct
MGVLLTQRTLLSLVLALLFPSMASMAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 383 aa, synthetic construct
MGVLLTQRTLLSLVLALLFPSMASMAMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 357 aa, synthetic construct [sequence omitted]

SEQ ID NO: 4 - protein, 357 aa, synthetic construct
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK